SEQUENCE LISTING (23 sequences)

SEQ ID NO: 1 - protein, 16 aa, Drosophila melanogaster
RQIKIWFQNRRMKWKK

SEQ ID NO: 2 - protein, 13 aa, Human immunodeficiency virus
GRKKRRQRRRPPQ

SEQ ID NO: 3 - protein, 27 aa, Artificial
  Artificial delivery peptide
GWTLNSAGYLLGPINLKALAALAKKIL

SEQ ID NO: 4 - protein, 34 aa, herpes simplex virus 7
DAATATRGRSAASRPTERPRAPARSASRPRRPVE

SEQ ID NO: 5 - protein, 18 aa, Artificial
  Artificial amphiphilic peptide 6
KLALKLALKALKAALKLA

SEQ ID NO: 6 - protein, 27 aa, Artificial
  Artificial sSignal sequence based peptide I
GALFLGWLGAAGSTMGAWSQPKKKRKV

SEQ ID NO: 7 - protein, 16 aa, Artificial
  Artificial signal sequence based peptide II
AAVALLPAVLLALLAP

SEQ ID NO: 8 - protein, 7 aa, Simian virus 40
PKKKRKV

SEQ ID NO: 9 - protein, 4 aa, Artificial
  Artificial platelet activating factor receptor of neutrophils
MLFY

SEQ ID NO: 10 - protein, 15 aa, Artificial
  Artificial delivery peptide based on Fragile X related proteins
PQRRNRSRRRRFRGQ

SEQ ID NO: 11 - protein, 7 aa, Artificial
  Artifical delivery peptide based on angiogenin L
IMRRRGL

SEQ ID NO: 12 - protein, 11 aa, Human immunodeficiency virus
LQLPPLERLTL

SEQ ID NO: 13 - protein, 11 aa, Artificial
  Artificial delivery peptide based on PKI- oc
ELALKLAGLDI

SEQ ID NO: 14 - protein, 11 aa, Artificial
  Artificial delivery peptide based on MAPKK
DLQKKLEELEL

SEQ ID NO: 15 - protein, 12 aa, Artificial
  Artificial delivery peptide based on actin I
ALPHAIMRLDLA

SEQ ID NO: 16 - protein, 7 aa, Simian virus 40
PKKKRKV

SEQ ID NO: 17 - protein, 13 aa, Artificial
  Artifical delivery peptide based on dermaseptin
ALWKTLLKKVLKA

SEQ ID NO: 18 - protein, 4 aa, Artificial
  Artificial ER localization signal peptide
KDEL

SEQ ID NO: 19 - DNA, 11 bp, Artificial
  Artificial oligonucleotide that can be conjugated to cholesterol derivatives
aggtcgggatg

SEQ ID NO: 20 - DNA, 18 bp, Artificial
  Artificial oligonucleotide that can be conjugated to cholesterol derivatives
gagcgcgaggtcgggatg

SEQ ID NO: 21 - DNA, 11 bp, Artificial
  Artificial oligonucleotide conjugate
gtagggctgga

SEQ ID NO: 22 - DNA, 18 bp, Artificial
  Artificial oligonucleotide conjugate
gtagggctggagcgcgag

SEQ ID NO: 23 - DNA, 19 bp, Artificial
  Artificial oligonucleotide that is a 2′-MOE phosphorothioate gapmer
ccaucccgacgtgcgcucc

(19) United States
(12) Patent Application Publication
(10) Pub. No.: US 2008/0199960 A1
(43) Pub. Date: Aug. 21, 2008

(54) Methods for the Delivery of Oligomeric Compounds

(76) Inventors: Rudolph L. Juliano, Chapel Hill, NC (US); Hyunmin Kang, Chapel Hill, NC (US); Anna Astriab-Fisher, Chapel Hill, NC (US); Piet Herdewijn, Wezemaal (BE); Patrick Chaltin, Geldenaken (BE); Arthur Van Aerschot, Heist-op-den-Berg (BE)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 11/596,238

(22) PCT Filed: May 13, 2005
(86) PCT No.: PCT/US05/16746

§ 371 Date: Jan. 14, 2008

Related U.S. Application Data
(60) Provisional application No. 60/570,796, filed on May 13, 2004.

(30) Foreign Application Priority Data
Apr. 22, 2005 (GB) 0508114.6

(51) Int. Cl. C12N 15/00; C12N 5/00

(52) U.S. Cl. 435/440; 435/325

(57) ABSTRACT

The presently disclosed subject matter relates to the delivery of oligonucleotides to cells through the delivery of a composition or reagent comprising a hybridization complex comprising a first antisense oligonucleotide which is modified to have a higher stability against degradation, and a second sense oligonucleotide which is prone to degradation. The presently disclosed subject matter furthermore relates to dendrimeric bioconjugates and compositions or reagents comprising them, wherein the bioconjugate comprises a conjugate moiety coupled to a dendrimeric structure and to their use to deliver oligomeric compounds including oligonucleotides or duplexes, as described above, to cells for modulation of gene expression (i.e. antisense or antigene therapy/research, RNA interference).

### CROSS REFERENCE TO RELATED APPLICATIONS

This application is based on and claims priority to U.S. Provisional Application Ser. No. 60/570,796, filed May 13, 2004, and United Kingdom Patent Application No. 0508114.6, filed Apr. 22, 2005, the disclosures of which are herein incorporated by reference in their entireties.

### TECHNICAL FIELD

The presently disclosed subject matter relates generally to the delivery of oligonucleotides to cells through the delivery of a composition or reagent comprising a hybridization complex comprising a first antisense oligonucleotide which is modified to have a higher stability against degradation, and a second sense oligonucleotide which is prone to degradation. The presently disclosed subject matter furthermore relates to dendrimeric bioconjugates and compositions or reagents comprising them, wherein the bioconjugate comprises a conjugate moiety coupled to a dendrimeric structure and to their use to deliver oligomeric compounds including oligonucleotides or duplexes, as described above, to cells for modulation of gene expression (i.e., antisense or antigene therapy/research, RNA interference).

### BACKGROUND ART

Advances in the field of biotechnology have led to significant advances in the treatment of diseases such as cancer, genetic diseases, arthritis, etc. and in the way research is performed. Many such advances involve the administration of oligonucleotides and other nucleic acids to a subject, particularly a human subject in order to modulate the expression of genes.

Modulation of gene expression is an extremely important and widely used research tool and constitutes a potent method for treating or preventing many diseases. This modulation of gene expression can be achieved in multiple ways. The strategies called “antisense”, “antigene”, or “RNA-interference” can be applied. These approaches utilize, for example, antisense nucleic acids, ribozymes, triplex agents or siRNAs to block transcription or translation of a specific mRNA or DNA of a target gene, either by masking that mRNA with an antisense nucleic acid or DNA with a triplex agent, by cleaving the nucleotide sequence with a ribozyme or by destruction of the mRNA through a complex mechanism involved in RNA-interference. In all the above-mentioned strategies, mainly oligonucleotides are used as effector compounds, although also small molecules and other structures are applied.

All the oligonucleotide-based strategies for modulating gene expression have a huge potential for the therapeutic manipulation of gene expression, but pharmacological applications of oligonucleotides have been hindered mainly by the effective delivery of these compounds to their sites of action within cells.

Focusing on the antisense strategy, antisense oligonucleotides are very powerful tools for inhibition of gene expression (Scherer, L. J., and Rossi, J. J. (2003) Nat. Biotechnol. 21, 1457-1465). The initial hurdles of enzymatic instability and poor hybridization strength of natural antisense oligonucleotides have been largely overcome by introducing chemical modifications of oligonucleotides used. This has made the antisense strategy very attractive for therapeutic manipulation of the gene expression. However, delivery of oligonucleotides to their sites of action within cells in vivo is the major bottleneck for the widespread use of antisense oligonucleotides in clinical applications (Shoji, Y., and Nakashima, H. (2004) Curr. Pharm. Des. 10, 785-796.). It has been demonstrated that, at the best, no more than 6-12% of oligonucleotides targeting an RNA sequence are efficient at forming the duplex necessary for the antisense effect (Herdewijn P. et al. Antisense Nucleic Acids Drug Dev 2000, 10, 297-310).

A process called RNA-interference evolved recently as a widely used technology. It has been discovered that RNA viruses replicate through double-stranded intermediates that are cleaved by a cytosolic enzyme (DICER) into short RNAs with a specific structure: two 21-nucleotide strands of RNA with 19 complementary nucleotides of dsRNA and two unpaired 3′-nucleotides at either end called short interfering RNA (siRNA). The antisense strand of this siRNA duplex is then assembled into a multi-protein complex called RISC (RNA-induced silencing complex). One of the proteins of the complex catalitically degrades the viral mRNA, thus eliminating the source of infection. siRNA thus results when transposons, viruses, or endogenous genes express long double-stranded RNA, or when double-stranded RNA is introduced experimentally into plant and animal cells to trigger gene silencing, a process known as RNA interference. Interestingly, synthetic short double-stranded RNA stretches (siRNA; 21-23 nt) with a specific mRNA target sequence were also found to have the capacity to mediate the degradation of mRNA in a specific way.

For RNAi to be successful, problems like specificity, production capacity, and delivery to the host cells need to be improved. The delivery aspect of siRNA is much more related to antisense oligodeoxyribonucleotides than to plasmid DNA, meaning that a delivery reagent developed for DNA is most likely not suitable for siRNA. Until now, synthetic siRNAs are most effective when delivered as double strands, whereas antisense oligonucleotides are delivered as single strands.

Improved delivery techniques for oligonucleotides are widely being investigated and comprise, amongst others, biochemical microinjection, liposomes-mediated oligonucelotide delivery, oligonucleotide-conjugates (with groups like cholesterol, cell-penetrating peptides, or ligands for cellular receptors), polyethylenimine-mediated delivery, polymer delivery, the use of biodegradable microparticles and electroporation.

One of the potential solutions for this delivery problem is the use of liposomes (Boulikas, T. (1996) Oncology Reports 3, 989-995). The antisense oligonucleotide can be dissolved in the hydrophilic inside of the vesicles (this is in contradiction with the formation of multilamellar structures described in the next paragraph) and delivered to the cell by fusion of the vesicle with membranes. However, the number of oligonucleotides that can be locked into such a vesicle is low. The delivery process can be improved by conjugation of the antisense oligonucleotide with cholesterol (Bijsterbosch, M. K. et al. (2001) Biochem., Pharmacol 62, 627-633.) or by the use of cationic lipids for liposome formulation (Weisman, S. et al. (2004) Biophys. J. 87, 609-614). A disadvantage of the first approach is that the cholesterol moiety is covalently attached to the oligonucleotide which influences the intracellular distribution of the antisense oligonucleotide.

Also dendrimers have been applied for increasing the uptake of oligonucleotides into cells. PAMAM dendrimers are cationic polymers that have considerable effectiveness in gene transfer or oligonucleotide delivery at the cell (H. Yoo and R. L. Juliano Nucleic Acids Research, 2000, Vol. 28, No. 21, 4225-4231).

However, the delivery of oligonucleotides to their target is still not optimal and leads to problems for the therapeutic application of the oligonucleotide-based strategies, such as the antisense therapy.

### SUMMARY

The presently disclosed subject matter relates to the delivery of oligomeric compounds to cells, especially oligonucleotides, through the delivery of a composition or a reagent or a hybridization complex which comprises a first (antisense) oligonucleotide which is modified to have a higher stability against degradation, and a second (sense) oligonucleotide, which is prone to degradation. The first and second oligonucleotides are not covalently linked, but hybridize with each other.

A first aspect of the presently disclosed subject matter therefore relates to a reagent or (pharmaceutical) composition comprising a hybridization complex or a duplex of oligonucleotides which comprise (i) a first oligonucleotide strand, wherein the first oligonucleotide strand is a (partially or fully) modified oligonucleotide that hybridizes to at least a portion (region, segment or site) of a target nucleic acid, more in particular a mRNA molecule transcribed by a specific gene and (ii) a second oligonucleotide strand, wherein the second oligonucleotide strand comprises an oligonucleotide, more in particular a DNA-oligonucleotide (non-modified) that is complementary, in a specific embodiment 100% complementary, to at least a part of the first oligonucleotide strand. Another aspect of the presently disclosed subject matter relates to the use of said hybridization complex or compositions comprising the complex as a research tool, as a medicine, in a diagnostic kit or for the manufacture of a medicament for the prevention or treatment of a specific disorder. Yet another aspect of the presently disclosed subject matter relates to a method of inhibiting the expression of a gene in a cell, the method comprising administering to the cell a hybridization complex or a reagent or (pharmaceutical) composition comprising a hybridization complex, wherein the hybridization complex comprises (i) a first oligonucleotide strand, wherein the first oligonucleotide strand is a fully or partially modified oligonucleotide that hybridizes to at least a portion of a target nucleic acid; and (ii) a second oligonucleotide strand, wherein the second oligonucleotide strand comprises a DNA-oligonucleotide that is complementary, in a specific embodiment is 100% complementary, to at least a part of the first strand, whereby the first oligonucleotide strand and the second oligonucleotide strand enter the cell and the expression of the gene in the cell is modified, more specifically inhibited. Another aspect of the presently disclosed subject matter relates to a reagent or (pharmaceutical) composition comprising the hybridization complex.

In a particular embodiment, the presently disclosed subject matter relates to a method of inhibiting the expression of a gene in a cell through the antisense strategy, the method comprising administering to the cell a hybridization complex as described herein, wherein the oligonucleotide has an antisense application, thus interacts with mRNA transcribed from a gene and is used for the antisense strategy. The method provides in a particular embodiment for the delivery of oligonucleotides for antisense applications.

In a particular embodiment, the first oligonucleotide is modified for a certain percentage between 10% and 90% or 100% and can thus be 10%, 20%, 30%, 40%, 50%, 60%, 70%, 80%, 90% or 100% or everything therein between, more in particular is modified between 20% and 90%, or is modified between 30%, 40% or 50% and 90% or 100%.

In a particular embodiment of the presently disclosed subject matter, the first oligonucleotide strand comprises at least one ribonucleotide, which can be a modified ribonucleotide and yet in another particular embodiment, the first oligonucleotide comprises a certain percentage of ribonucleoitdes which can be between 10% and 90% or 100% and can thus be 10%, 20%, 30%, 40%, 50%, 60%, 70%, 80%, 90% or 100%. In another particular embodiment, the first oligonucleotide is a RNA-oligonucleotide.

Yet in another embodiment, the first oligonucleotide has a length of between 10 and 30 nucleotides, yet more in particular between 15 and 25 nucleotides and yet more in particular has 16, 17, 18, 19, 20, 21, 22, 23, 24 or 25 nucleotides.

In another particular embodiment, the presently disclosed subject matter relates to the hybridization complex, to the use thereof or to methods using said complexes, wherein the first oligonucleotide strand comprises a modification selected from the group consisting of a internucleoside linkage modification, a carbohydrate modification or a nucleobase modification. In yet another particular embodiment, the internucleoside modification comprises one or more phosphorothioate linkages. Still another embodiment, the nucleotide modification comprises a 2′ modification of the carbohydrate, which is in particular selected from the group consisting of 2′-halo, 2′-O-methyl, 2′-O-ethyl, 2′-O-propyl, 2′-O-butyl; 2′-O-pentyl, 2′-O-(2-methoxyethyl), and 2′-O-[2-[N,N-dimethylamino)oxy]-ethyl], or more in particular, is a 2′-O-methyl group. In another particular embodiment, the 2′ modification comprises a locked nucleic acid, wherein the locked nucleic acid is characterized by a methylene bridge that connects a 2′-oxygen with a 4′-carbon of a ribose.

In a particular embodiment, the first oligonucleotide contains only deoxyribonucleotides or only ribonucleotides or a combintaion of ribonucleotides and deoxyribonucleotides, which can than be modified in the internucleotide linkages (for example a phopshorothioate linkage) or the carbohydrate and/or the nucleobase can be modified.

In another particular embodiment, the first oligonucleotide is a chimeric oligonucleotide and more in particular is a gapmer. In yet another embodiment, the first oligonucleotide gapmer strand has the following general structure:

(2′-O-modified ribonucleotides)ₓ-(deoxyribonucleotides)_{y}-(2′-O-modified ribonucleotides)_{z}, wherein y is at least 5 and x+y+z equals at least 18 but is less than 30, or more inparticular, wherein y equals 5, and x and z differ from each other by no more than 2 and yet more in particular, wherein y is at least 7.

The second oligonucleotide strand of the hybridizationcomplex has to be an easily degradable oligonucleotide such as a naturally occuring phosphodiester comprising deoxyribonucleic acid, but can be of another structure as long as it is degraded in a duplex form and more easily degraded that the first oligonucleotide.

In another embodiment, the presently disclosed subject matter relates to the hybridization complex, to the use thereof or to methods using said complexes, wherein the second oligonucleotide strand is shorter than or equal in length to the first oligonucleotide strand. In another particular embodiment, the second oligonucleoitde strand of the hybridization complex is shorter, so comprises less nucleotides than the first oligonucleotide strand, yet more in particular is 1, 2, 3, 4, 5, 6, 7 or 8 nucleotides shorter. In a particular embodiment, the first oligonucleotide strand is a 20-mer while the second oligonucleotide strand is a 16-mer or 18-mer.

In another particular embodiment, the second oligonucleotide strand is an unmodified deoxyribonucleic acid.

In another embodiment of the presently disclosed subject matter, the first and/or second oligonucleotide strands of the hybridization complex are conjugated to one or more conjugate moieties. In a particular embodiment, only the second oligonucleotide strand is conjugated to a conjugate moiety, while in another embodiment, only the first oligonucleotide strand is conjugated to a conjugate moiety, while in another embodiment, both oligonucleotide strands are conjugated to a conjugate moiety. In a particular embodiment, the conjugate moiety is coupled at the 5′-end or 3′-end. Thus, in a particular emboidment the oligonucleotide strand further comprises a modification selected from the group consisting of a 5′-end modification and a 3′-end modification. The conjugate moiety can be selected from lipophilic molecules including steroid molecules such as cholesterol; proteins (e.g., antibodies, enzymes, serum proteins); peptides such as Tat-peptide; vitamins (water-soluble or lipid-soluble); polymers (water-soluble or lipid-soluble, including dendrimers); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonuclease/intercalators; crosslinking agents (e.g., photoactive, redox active), and combinations and derivatives thereof. In a further embodiment, the said 3′-end or 5′-end modification, comprises a conjugated cholesterol moiety or a conjugated peptide.

In another particular embodiment, the second oligonucleotide strand of the hybridization complex is coupled to one or more (2, 3, 4, 5, 6, 7, 8, etc.) oligonucleotides through linkers in order to form a dendrimeric structure and this dendrimeric structure is further conjugated to a conjugate moiety. The oligonucleotides of the dendrimeric structure all function as second sense oligonucleotide strand and can hybridize with first oligonucleotides and thus multiple hybridization complexes are so coupled in a dendrimeric structure.

In a particular embodiment, the oligonucleotides of the dendrimer are identical or the dendrimerc contains two or more different sequences of oligonucleotides, in order to deliver multiple first oligonucleotide strands of different sequences and targeted to different nucleic acids or different regions, segments or sites of nucleic acid targets.

Another aspect of the presently disclosed subject matter relates to a method of enhancing the uptake of a single-stranded oligonucleotide strand by a cell, the method comprising (a) hybridizing to the first single-stranded oligonucleotide a second oligonucleotide (deoxyribonucleic acid molecule) that is 100% complementary to a subsequence of the single-stranded oligonucleotide to create a double-stranded molecule; and (b) contacting the cell with the double-stranded molecule, whereby uptake of the single-stranded oligonucleotide by the cell is enhanced.

In particular embodiments, the first and second oligonucleotides are as described hereinabove.

Another aspect of the presently disclosed subject matter provides a method of treatment of a certain disorder in a mammal comprising administering to the mammal in need of such treatment the reagent or composition comprising the hybridization complex as described herein, combined in a particular embodiment with a cationic lipid.

In another embodiment of the presently disclosed subject matter, the reagent or composition comprising a hybridization complex further comprises a cationic lipid, such as liposomes or lipofectamine, yet more in particular lipofectamine 2000.

Another aspect of the presently disclosed subject matter provides for a pharmaceutical composition comprising a hybridization complex as described herein in admixture with at least a pharmaceutically acceptable carrier, in a particular embodiment further comprising a cationic lipid.

In another particular embodiment, the reagent or composition further comprises a dendrimeric bioconjugate comprising a conjugate moiety covalently linked to a dendrimeric structure. The conjugate moiety of the dendrimeric bioconjugate can be selected from lipophilic molecules including steroid molecules such as cholesterol; proteins (e.g., antibodies, enzymes, serum proteins); peptides such as Tat-peptide; vitamins (water-soluble or lipid-soluble); polymers (water-soluble or lipid-soluble); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonuclease/intercalators; crosslinking agents (e.g., photoactive, redox active), and combinations and derivatives thereof. In a particular embodiment, the conjugate moiety of the dendrimeric bioconjuagte is selected from lipophilic molecules, more in particular cholesterol and peptides, more in particular Tat-peptide. The dendrimeric structure can comprise at least two oligonucleotides covalently attached to each other through a linker (dendrimeric oligonucleotide structure) or is a cationic polymer such as PAMAM dendrimers. Thus, in another embodiment of the presently disclosed subject matter, the second oligonucleotide strand of the hybridization complex is part of a dendrimeric structure comprising at least two sense-DNA-oligonucleotides and the dendrimeric structure is linked to a conjugate moiety. In another embodiment of the presently disclosed subject matter, the reagent comprises dendrimeric bioconjugates wherein the dendrimeric bioconjugate comprises a dendrimeric structure of at least two hybridization complexes covalently linked to each other through their second sense-DNA-oligonucleotide strands.

Another aspect of the presently disclosed subject matter thus relates to dendrimeric bioconjugates, wherein the bioconjugate dendrimers comprise a dendrimeric structure covalently linked to a conjugate moiety. In a particular embodiment, the conjugate moiety is selected from cholesterol or a peptide (i.e. Tat-peptide). This dendrimeric bioconjugate can be used for the delivery of oligomeric compounds, more in particular oligonucleotides, duplexes like the hybridization complex, vectors, siRNAs, etc. to cells for the modulation of gene expression and thus the presently disclosed subject matter relates to the use of the dendrimeric bioconjugate for the in vitro or in vivo inhibition of transcription or translation of a gene, for example as a research tool. The presently disclosed subject matter relates also to the use of the dendrimeric bioconjugates as a medicine and for the manufacture of a medicament for the treatment or prevention of a specific disease. The presently disclosed subject matter also relates to the process for preparing the dendrimeric bioconjugates. Another aspect of the presently disclosed subject matter relates to a method of inhibiting the expression of a gene in a cell, the method comprising administering to the cell a reagent or composition comprising a dendrimeric bioconjugate and an oligomeric compound, wherein the dendrimeric bioconjugate comprises a dendrimeric structure covalently linked to a conjugate moiety, whereby the oligomeric compound modulates the expression of the gene in the cell. In a particular embodiment, the composition furter comprises a cationic lipid. Another aspect relates to a method of enhancing the uptake of an oligomeric compound by a cell, the method comprising (i) combining the oligomeric structure with a dendrimeric bioconjugate in a composition as described herein; and (ii) contacting the cell with the composition, whereby uptake of the oligomeric compound by the cell is achieved or enhanced. In a particular embodiment, a step ensuring hybridization or interaction between the oligomeric compound and the dendrimeric structure is provided.

The presently disclosed subject matter also provides a pharmaceutical composition comprising the dendrimeric bioconjugate and an oligomeric compound in admixture with at least a pharmaceutically acceptable carrier, in a particular embodiment further comprising a cationic lipid, such as lipofectamine. Another aspect of the presently disclosed subject matter provides a method of treatment of a certain disorder in a mammal comprising administering to the mammal in need of such treatment a composition comprising a bioconjugate dendrimer and an oligomeric compound as described herein, combined in a particular embodiment with a cationic lipid such as lipofectamine.

In a particular embodiment, the dendrimeric bioconjugate comprises cholesterol as conjugate moiety and a dendrimeric oligonucleotide structure as dendrimeric structure and yet is more in particular the dendrimeric bioconjugate as specified herein, namely the compounds 11 or 13 coupled to oligonucleotides as in compounds 12a, 12b, 14a or 14b. In another embodiment, the dendrimeric bioconjugate comprises a peptide as conjugate moiety and a dendrimeric oligonucleotide structure or cationic polymer such as PAMAM as dendrimeric structure.

In another particular embodiment, the oligomeric compounds in the composition comprising a dendrimeric bioconjugate have identical sequences. In another embodiment, the oligomeric compounds in the composition have different nucleotide sequences and comprise 2 or 3 or 4 or more different oligomeric compounds. Thus, the presently disclosed subject matter relates to a reagent or composition comprising a dendrimeric bioconjugate and an oligomeric compound or multiple different oligomeric compounds. The oligomeric compounds may be natuural or may be modified, while in a preferred embodiment, the oligomeric compounds of the composition comprising a dendrimeric bioconjugate are modified. Yet in another particular embodiment, the oligomeric compound in the composition comprising a bioconjugate dendrimer is selected from the group of single- or double-stranded oligonucleotides (i.e. DNA-oligonucleotides, RNA-oligonucleotides or oligonucleotides suitable for RNA-interference such as siRNA, shRNA and microRNA) or vectors encoding the same, oligonucleotide analogs, duplex oligonucleotides such as hybridization complexes and the oligomeric compound can be modified and/or conjugated to a conjugate moiety. Yet in another embodiment, the oligomeric compound in the bioconjuagte dendrimeric comprising composition is an oligonucleotide with a length of between 10 and 30 nucleotides, yet more in particular between 15 and 25 nucleotides and yet more in particular has 16, 17, 18, 19, 20, 21, 22, 23, 24 or 25 nucleotides.

In a particular embodiment, the dendrimeric structure is a dendrimeric oligonucleotide structure. The number of oligonucleotides in the dendrimeric oligonucleotide structure can be 2, 3, 4, 5, 6 or more, and is in a particular embodiment 2, 4, 8, 16 or more. The oligonucelotides are covalently attached to each other through linking moieties (linkers). In a particular embodiment, the length of the oligonucleotides of the dendrimeric oligonucleotide structures is between 8 and 30 nucleotides, and can be 8, 9, 10, 11, 12, 13 and more and is yet more in particular between 10 and 25 nucleotides and yet more in particular has 16, 17, 18, 19, 20, 21, 22, 23, 24 or 25 nucleotides and the length of the oligonucleotides within one dendrimeric oligonucleotide strucure do not have to be the same (also depending on synthesis). The oligonucleoitdes can be shorter than the oligomeric compounds they hybridize with as described herein and can have between 1 to 15 or 12 or 10 or 8 nucleotides less or can have 1, 2, 3, 4, 5, 6, 7, 8, 9, 10, 11, 12 or more nucleotides less than the antisense oligomeric compound. In a particular embodiment, the oligonucleotides within one dendrimeric oligonucleotide structure have a different sequence and they can have 2 or 3 or 4 or more different nucleotide sequences. The dendrimeric structure comprises in a particular embodiment DNA-oligonucleotides, while in a particular embodiment the DNA-oligonucleotides are hybridized to complementary oligonucleotide strands comprising modified nucleotides. Thus, in a particular embodiment, the presently disclosed subject matter relates to a composition comprising a bioconjugate dendrimer and partially or fully modified oligonucleotides wherein the oligonucleotides are hybridized with the dendrimeric structure.

Another aspect of the presently disclosed subject matter provides for methods for preparing the bioconjugate dendrimers such as the dendrimeric oligonucleotide bioconjugate and for the preparation of cholesterylacetic acid. In some embodiments, the method for preparing the cholesterol-oligonucleotide dendrimeric bioconjugate comprises (a) derivatizing cholesterol in order to obtain a free carboxyl group; (b) coupling to the derivatized cholesterol Fmoc-lysine-OMe or an analoguous structure comprising a protected amino group and a protected carboxyl group which are separately cleavable; (c) deprotecting the carboxylgroup; (d) coupling the obtained compound to a solid phase through an acid labile function, like an ester; (e) deprotecting the amino group; (f) repetitive steps of [1] coupling of a compound with two protected amino functions and a free carboxylgroup like (Fmoc)₂-Lys-OH and [2] deprotection of the amino-funtion protecting groups according to the amount of dendrimeric units requested; (g) coupling a linker with a free hydroxyl as reactive group; and (h) performing standard oligonucleotide synthesis followed by cleaving the compound from the solid phase. For obtaining a monomer, step (f) can be dropped.

For the derivatization of cholesterol to contain a free carboxyl group, cholesterolacetic acid was prepared. In some embodiments, a method for preparing cholesterolacetic acid comprises: (a) reacting cholesterol with a acetalic protected halogenacetaldehyde (such as bromoacetaldehyde diethylacetal in order to create cholesterylacetaldehyde diethylacetal); (b) deprotecting the acetale protecting groups to cholesterolacetaldehyde; and (c) oxidizing the aldehyde to a carboxylic acid, namely cholesterylacetic acid.

Another aspect of the presently disclosed subject matter relates to the use of dendrimeric structures such as cationic polymers such as PAMAM for the delivery of oligomeric compounds, more in particular single- or double-stranded oligonucelotides, for RNA interference. The presently disclosed subject matter thus relates to a method of inhibiting the expression of a gene in a cell through RNA-interference, the method comprising administering to the cell a composition comprising a dendrimeric structure such as PAMAM and an oligomeric compound for RNA-interference, more in particular with a siRNA or shRNA. The presently disclosed subject matter also relates to a pharmaceutical composition comprising a dendrimerci structure and oligomeric compound for RNA-interference, more specifically siRNA or shRNA or microRNA, in admixture with a pharmaceutically acceptable carrier.

### BRIEF DESCRIPTION OF THE DRAWINGS

FIG. 1 depicts the concept of double stranded delivery of antisense oligonucleotides. FIG. 1A: delivery of double stranded hybrids complexed with Lipofectamine 2000 into the cytoplasm (unmodified strand: red; modified stand: green). FIG. 1B: enzymatic degradation of the non-modified strand. FIG. 1C: transport of the modified strand into the nucleus.

FIG. 2 depicts flow cytometry analysis of the cellular uptake of the antisense oligonucleotides in single and double stranded format. SS: single stranded 20-mer antisense; ds14-mer: double stranded duplex antisense 20-mer and sense 14-mer DNA; ds16-mer: double stranded duplex antisense 20-mer and sense 16-mer DNA; dsl8-mer: double stranded duplex antisense 20-mer and sense 18-mer DNA.

FIG. 3 depicts toxicity of antisense oligonucleotides in single and double stranded format. No: no treatment; SS: single stranded 20-mer antisense; ds16-mer: double stranded duplex antisense 20-mer and sense 16-mer DNA strand; ds18-mer: double stranded duplex antisense 20-mer and sense 18-mer DNA.

FIG. 4 depicts confocal microscopy analysis of cellular distribution of the antisense 20-mer/sense 18-mer duplex 24 hours after treatment. Sense strand: red; antisense strand: green.

FIG. 5 depicts flow cytometry analysis of specificity of inhibition of the P-glycoprotein expression. Double stranded delivery approach at 0.1 μM concentration (untr: untreated; ds mm 18-mer: double stranded duplex, antisense mismatch 20-mer with sense 18-mer DNA; ds 18-mer: double stranded duplex antisense 20-mer with sense 18-mer DNA).

FIG. 6 depicts concentration-dependent inhibition of P-glycoprotein expression using antisense oligonucleotide. NO: no treatment; SS: single stranded 20-mer antisense; DS-18-mer: double stranded duplex with antisense 20-mer and sense 18-mer DNA.

FIG. 7: Scheme representing the synthesis of cholesterol conjugated lysine on solid support.

i: BrCH₂CH(OEt)₂, NaH, DMF/THF; ii: CF₃COOH aqueous in DCM; iii: CrO₃, H₂SO₄, CH₃COCH₃, H₂O; iv: Fmoc-Lysine-OMe HCL, HATU, DIEA, DMF; v: LiOH, THF, H₂O; vi: HBTU, HOBT, DIEA, DMF.

FIG. 8: Scheme synthesis of cholesterol modified oligonucleotide monomers.

i: piperidine, DMF; ii: MMTrO(CH₂)₃COOH, HOBt, HBTU, DIEA, DMF; iii: CCl₃COOH, DCM; iv: oligonucleotide (GAGCGCGAGGTCGGGATG; SEQ ID NO: 20) synthesis; v: deprotection (aq. CH₃NH₂—NH₃) 10b: same molecule with oligo sequence: AGGTCGGGATG (SEQ ID NO: 19).

FIG. 9: Scheme representing the synthesis of cholesterol modified oligonucleotide dimeric dendrimer.

i: piperidine, DMF; ii: (Fmoc)₂Iys-OH, HBTU, HOBT, DIEA, DMF; iii: MMTrO(CH₂)₃COOH, HOBT, HBTU, DIEA, DMF; iv: CCl₃COOH, DCM; v: oligonucleotide (GAGCGCGAGGTCGGGATG; SEQ ID NO: 20) synthesis; vi: deprotection (aq. CH₃NH₂—NH₃)

12b: same construct with oligo sequence: AGGTCGGGATG (SEQ ID NO: 19).

FIG. 10: Scheme representing the synthesis of cholesterol modified oligonucleotide tetrameric dendrimer.

i: piperidine, DMF; ii: (Fmoc)₂Iys-OH, HBTU, HOBT, DIEA, DMF; iii: MMTrO(CH₂)₃COOH, HOBT/HBTU, DIEA, DMF; iv: CCl₃COOH, DCM; v: oligonucleotide (GAGCGCGAGGTCGGGATG; SEQ ID NO: 20) synthesis; vi: deprotection procedure (aq. CH₃NH₂—NH₃)

14b: same construct with oligo sequence: AGGTCGGGATG (SEQ ID NO: 19).

FIG. 11. Overview of monomeric and dendrimeric oligonucleotides (DNA) that were used to concentrate antisense oligonucleotides in cationic lipids for cellular delivery (as well 11-mer (SEQ ID NO: 21) as 18-mer (SEQ ID NO: 22) oligonucleotides were evaluated as complexing agent for the antisense oligonucleotide).

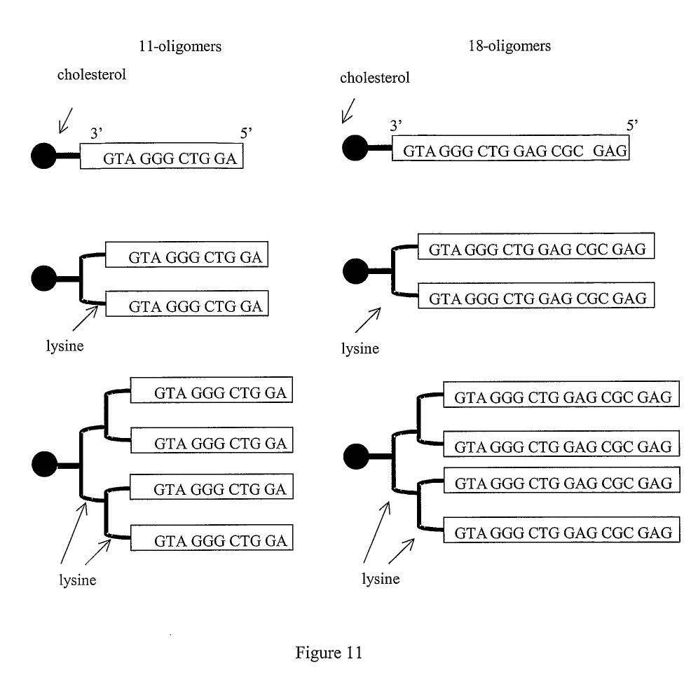

FIG. 12. Structure of antisense oligonucleotide (SEQ ID NO: 23) directed at mRNA that is transcibed into P-glycoprotein.

FIG. 13. Cholesterol-Oligo complex 24 hour transfection ±Lipofectamine 2000 in 5% FBS.

a) Control; b) Lipofectamine 2000; c) control inhibitory antisense oligonucleotide 50 nM+Lipofectamine 2000; d). 12a 1× 50 nM+Lipofectamine 2000; e) 14a 1× 50 nM+Lipofectamine 2000; f) 14a 1× 50 nM+Lipofectamine 2000; g) 12a 2× 50 nM+Lipofectamine 2000; h) 14a 4× 50 nM+Lipofectamine 2000; i) 14a 4× 50 nM+Lipofectamine 2000.

FIG. 14. Preparation of PAMAM G5 conjugate of BODIPY and TAT (PBT).

FIG. 15. Delivery of siRNA with PAMAM Dendrimers for P-glycoprotein expression inhibition. PAMAM derivatives (P or BPT, 30 μg/ml) or Lipofectamine 2000 (L2, 2 μg/ml) were complexed to an siRNA (ORF1, 100 nM) for transfection into NIH 3T3 MDR cells. P-glycoprotein cell surface expression in the viable cells was evaluated as described herein. X-axes represent fluorescence intensity and y-axes represent cell number. The demarcations between the left and right boxes in the figures are set at one standard deviation below the mean of the untreated control.

FIG. 16. Delivery of MDR1 antisense with PAMAM-TAT dendrimer for P-glycoprotein expression inhibition. PAMAM derivatives (P or BPT, 30 μg/ml) or Lipofectamine 2000 (L2, 2 μg/ml) were complexed to an antisense (5995, 100 nM) for transfection into NIH 3T3 MDR cells. P-glycoprotein cell surface expression in the viable cells was evaluated as described herein. X-axes represent fluorescence intensity and y-axes represent cell number. The demarcations between the left and right boxes in the figures are set at one standard deviation below the mean of the untreated control.

### DETAILED DESCRIPTION

Abbreviations used herein are explained or are known by the person skilled in the art or can be found easily by a person skilled in the art in literature.

The presently disclosed subject matter provides methods for the cellular delivery of oligomeric compounds, more in particular single stranded oligonucleotides, more in particular for antisense or antigene strategies or for RNA-interference as well as for other mechanism wherein an oligomeric compound, more in particular a single-stranded oligonucleotide needs to be delivered in the cell. The presently disclosed subject matter provides more in particular a method for the cellular delivery of oligomeric compounds, more in particular single stranded oligonucleotides through the delivery or use of a hybridization complex or a duplex comprising a first oligonucleotide, more in particular an antisense oligonucleotide, yet more in particular an antisense oligonucleotide for antisense applications, which is modified to have a higher stability against degradation, and a second sense oligonucleotide, which is prone to degradation. The presently disclosed subject matter also provides a method for the delivery of oliigomeric compounds to the cell through the use of bioconjugate dendrimers.

More in particular, for the antisense strategy, oligonucleotides for antisense applications are in the prior art delivered as single stranded molecules. The presently disclosed subject matter provides however for the double-stranded delivery of oligonucleotides for antisense applications with specifics for the oligonucleotides as described herein.

In this study we evaluated a new concept for delivery of oligonucleotides for antisense applications in cellular systems. We have shown that formation of a duplex between the active antisense oligonucleotide (with a chemically modified backbone) and an easily degradable complementary oligodeoxynucleotide, more in particular in the presence of liposomes such as Lipofectamine 2000, leads to better intracellular uptake and more significant pharmacological effect of the active oligonucleotide. Once the cellular uptake of the double stranded hybrid, the second sense oligonucleotide will be degraded and the antisense oligonucleotide will be able to exert its activity.

As an example, to evaluate our approach we targeted the MDRI gene, which coded for P-glycoprotein, a membrane ATPase associated with multi-drug resistance in tumor cells. The 2′-O-methyl gapmer antisense RNA (active component of the duplex) was complementary to a site flanking the AUG of the MDR1 message. Effective inhibition of P-glycoprotein expression was attained with sub-micromolar concentrations of duplexes under serum-replete conditions and was much stronger than with traditional single stranded antisense delivery. The results obtained show that double stranded delivery provides a simple and effective means for enhancing cellular uptake of pharmacologically active antisense oligonucleotides with the method or compounds of the presently disclosed subject matter.

Therefore, the presently disclosed subject matter relates to a method of enhancing the uptake of a single-stranded oligonucleotide strand by a cell, the method comprising:

(i) hybridizing to the first single-stranded oligonucleotide, which is in particular a RNA-oligonucleotide, a second oligonucleotide (deoxyribonucleic acid molecule) that is 100% complementary to a subsequence of the single-stranded oligonucleotide to create a double-stranded molecule (hybridization complex); and

(ii) contacting the cell with the double-stranded molecule;

whereby uptake of the single-stranded oligonucleotide by the cell is enhanced. The presently disclosed subject matter also provides a (pharmaceutical) composition comprising a hybridization complex as decribed herein and more in particular further comprises liposomesuch as lipofectamine 2000.

The first oligonucleotide has to be stable against degradation and therefore should be modified. Any oligonucleotide which is more or less stable to (enzymatic) degradation in cells or mammalian fluids can be used. It can have different lengths and different types of modifications. Normally it targets a specific nucleic acid target. It can contain deoxyribonucleotides or ribonucleotides or modifications thereof. In particular embodiments, the first oligonucleotide is an RNA-oligonucleotide, meaning that it contains nucleotides with 2′-substitutions, and this RNA-oligonucleotide is modified in its phosphate backbone or nucleoside. In a particular embodiment, the first oligonucleotide strand is a chimeric oligonucleotide and yet more in particular has the following general structure: (2′-O-modified ribonucleotides)ₓ-(deoxyribonucleotides)_{y}-(2′-O-modified ribonucleotides)_{z}, wherein y is at least 5 and x+y+z equals at least 18 but is less than 30, or more inparticular, wherein y equals 5, and x and z differ from each other by no more than 2 and yet more in particular, wherein y is at least 7.

Another aspect of the presently disclosed subject matter relates to a (pharmaceutical) composition or reagent comprising the hybridization complex, wherein the composition or reagent in a particular embodiment further comprises liposomes or other agents that aid the cellular delivery such as lipofectamine 2000.

The second oligonucleotide strand has to be prone to degradation and is therefore selected from DNA, although other oligonucleotides which are as degradable as DNA should also function in this method.

Methods for the determination of the stability of an oligonucleotide or oligomeric compound are known in the art and can be used to identify whether the oligonucleotides are stable or prone to degradation such as in Bishop J. S. et al. J. Biol. Chem. Volume 271, Number 10, 1996, 5698-5703.

As an example test method, the Serum or Cellular Stability Stability Assay as described hereunder can be used.

Serum stability assay: Aliquots of bovine serum (Life Technologies, Inc.) (1.5 ml) can be mixed with ³³P-labeled oligonucleotides (2 Ci/mmol; final concentration 1 μM) and incubated in screw-capped 1.5-ml polypropylene tubes at 37° C. At various time points ranging from 0 to 96 h, 150-μl aliquots can be removed and oligonucleotides extracted by the phenol/chloroform method as known in the art. The oligonucleotides can be precipitated with ethanol, solubilized in formamide-containing sample buffer, and fractionated by denaturing polyacrylamide gels as known in the art. Known amounts of ³³P-oligonucleotides can be run in parallel lanes as standards. The gels can be fixed in a 10% methanol plus 10% acetic acid solution, dried, and exposed to Kodak XAR-5 film for autoradiography. The radioactivity associated with each band on the gel can be quantified using a Fuji Bioimager and Fuji MacBas software.

In a particular embodiment, for the second oligonucleotide of the hybridization complex, also other than DNA-olignucleotides could be used, for example which are modified, but which are as sensitive (or maximally 10% less sensitive) or more sensitive to degradation as deoxyribonucleic acid in the Serum stability assay as described herein. The hybriidzatino capacity of the oligonucleotide needs to be present as can be determined in a melting temperature assay as known in the art.

The presently disclosed subject matter also relates to the approach of derivatisation/linking of the oligonucleotides of the hybridization complex with conjugate moieties (such as cholesterol or peptides). As an example the sense oligonucleotide starnd can be coupled to a conjugate moiety for delivering the (enzymatic) stable first oligonucleotide, but also the antisense strand could be coupled to a conjugate moiety. Therefore, in another example, the sense-strand of the hybridization complex was conjugated to a conjugation moiety, namely cholesterol. This approach/example has the advantage that the enzymatic stable antisense oligonucleotide itself is not derivatized (i.e. its intracellular distribution and hybridization with its target is not influenced by a reporter group). The cholesterol modified sense-DNA may stay locked in the endosomes and may be slowly degraded while the enzymatic more stable antisense oligonucleotide can diffuse into the cytoplasm and/or the nucleus.

Furthermore, a dendrimeric structure is used to deliver multiple antisense oligonucleotides through the duplex mechanism described herein and therefore a dendrimeric bioconjugate was constructed and used.

Antigene strategy, antisense strategy and RNA interference. Without intending to be bound by theory, single- and/or double-stranded oligonucleotides are believed to modulate gene expression by hybridizing to a nucleic acid target resulting in loss of normal function of the target nucleic acid (antisense or antigene strategies) or through a complex mechanism called RNA-interference. These approaches utilize for example antisense oligonucleotides, ribozymes, triplex agents or siRNAs to block transcription or translation of mRNA or DNA or of a specific mRNA or DNA, either by masking that mRNA with an antisense nucleic acid or DNA with a triplex agent, by cleaving the nucleotide sequence with a ribozyme or by destruction of the mRNA through a complex mechanism involved in RNA-interference.

Antisense nucleic acids or oligonucleotides are DNA or RNA molecules or nucleic acid analogs (e.g. hexitol nucleic acids, Peptide nucleic acids) that are complementary to at least a portion of a specific mRNA molecule (Weintraub Scientific American 1990; 262:40). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target cell. Also nucleic acids or analogs, complementary to the translation initiation site of a target gene/protein, e.g. between −10 or +10 regions of the nucleotide sequence, are preferred.

The potency of antisense oligonucleotides for inhibiting a target may be enhanced using various methods including addition of polylysine, encapsulation into liposomes (antibody targeted, cationic acid, Sendai virus derived, etc.) or into nanoparticles in order to deliver the oligonucleotides into cells. Other techniques for enhancing the antisense capacity of oligonucleotides exist, such as the conjugation of the antisense oligonucleotides for example to “cell penetrating peptides” (Manoharan, M. Antisense Nucleic Acid Drug Dev. 2002; 12(2): 103-128/Juliano, R.-L. Curr. Opin. Mol. Ther. 2000; 2(3): 297-303). However, the delivery of antisense oligonucleotides still constitutes a big problem for the field.

Throughout the text, the language “antisense oligonucleotide” is used normally to designate the sequence of an oligonucleotide, namely complementary or “antisense” compared to the sequence of the nucleic acid target or the “sense” oligonucleotide to which it can hybridize, while in some instances this terminology is used to designate antisense oligonucelotides used for antisense applications or strategy, but this will than be clear from the text and the description.

Use of for example an oligonucleotide or a PNA (Peptide nucleic acid) to stall transcription is known as the antigene strategy (e.g. triplex formation). In the case of oligonucleotides, the oligomer winds around double-helical DNA (major groove), forming a three-stranded helix. Therefore, these antigene compounds can be designed to recognise a unique site on a chosen gene and block transcription of that gene in vivo. (Maher et al. Antisense Res. and Dev. 1991; 1:227; Helene, C. Anticancer Drug Design 1991; 6:569/Casey, B. P. et al. Prog. Nucleic Acid Res. Mol. Biol. 2001; 67: 163-192/Pooga, M. et al. Biomol. Eng. 2001; 17(6): 183-192/Nielsen, P. E. Pharmacol. Toxicol. 2000; 86(1): 3-7). Antigene oligonucleotides as well as PNAs are easily synthesized by the man skilled in the art and are even commercially available.

Ribozymes are molecules possessing the ability to specifically cleave other single stranded RNA in a manner analogous to DNA restriction endonucleases. Through the modification of nucleotide sequences which encode these RNAs, it is possible to engineer molecules that recognise specific nucleotide sequences in an RNA molecule and cleave it (Cech J. Amer. Med. Assn. 1988; 260:3030). A major advantage of this approach is that, because they are sequence-specific, only mRNAs with particular sequences are inactivated.

There are two basic types of ribozymes namely, tetrahymena-type (Hasselhoff (1988) Nature 334:585) and “hammerhead”-type. Tetrahymena-type ribozymes recognise sequences which are four bases in length, while “hammerhead”-type ribozymes recognise base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

RNA-interference involves the insertion of small pieces of double stranded (ds) and even single stranded RNA into a cell. If the dsRNA corresponds with a gene in the cell, it will promote the destruction of mRNA produced by that gene, thereby preventing its expression. The technique has been shown to work on a variety of genes, even in human cells and in vivo. For example small interfering RNAs (siRNA), short-hairpin RNAs (shRNA), microRNA or vectors expressing such nucleic acids can be applied in the RNA-interference strategy in order to inhibit the translation of a target mRNA.

Typically, for RNA-interference, the combination of an antisense strand and a sense strand, each of which can be of a specified length, for example from 21 to 29 nucleotides long, is identified as a complementary pair of siRNA oligonucleotides which can contain modifications or substituents on their 3′ or 5′ ends. For RNA interference, also a single oligonucleotide having both the antisense portion as a first region in the oligonucleotide and the sense portion as a second region in the oligonucleotide can be used (hairpin oligonucleotide). The first and second regions are linked together by either a nucleotide linker (a string of one or more nucleotides that are linked together in a sequence) or by a non-nucleotide linker region or by a combination of both a nucleotide and non-nucleotide structure. In each of these structures, the oligonucleotide, when folded back on itself, would be complementary at least between the first region, the antisense portion, and the second region, the sense portion. Thus the oligonucleotide can have a palindrome within it structure wherein the first region, the antisense portion in the 5′ to 3′ direction, is complementary to the second region, the sense portion in the 3′ to 5′ direction.

The presently disclosed subject matter relates to the delivery of single-stranded oligonucleotides in the cell. This delivery can have any purpose, but in a particular embodiment is directed to the delivery of oligonucleotides for antisense applications, better called antisense strategies.

Target nucleic acid and modulation thereof. With the methods or compounds of the presently disclosed subject matter, oligomeric compound or single stranded oligonucleotides will be delivered in the cell and will be used in order to modulate the expression of a certain target gene or nucleic acid through the use of a hybridization complex or other delivery method. The single stranded oligonucleotides can hybridize with mRNA and modulate the translation of mRNA to proteins (blocking of translation or degradation of the taregt nucleic acid is obtained).

Therefore, the oligonucleotide that has to be delivered, namely the first oligonucleotide strand of the hybridization complex, has to be of the antisense sequence compared to the target nucleic acid or oligonucleotide (therefore it is named “antisense oligonucleotide”), not for 100% but always to a certain degree or percentage of a portion/region, segment or site of the target, as is known in the art. The second strand of the hybridization complex needs to be able to hybridize with the antisense first strand and therefore will need to have a sequence which is complementary to the antisesne strand, always to a certain degree or percentage as is known in the art (“sense oligonucleotide”).

As used herein, the term “target nucleic acid” or “nucleic acid target” is used to encompass any nucleic acid capable of being targeted including without limitation DNA, RNA (including pre-mRNA and mRNA or portions thereof) transcribed from such DNA, and also cDNA derived from such RNA. For antigene approaches, there is typically an interaction with DNA, while for the antisense approach, there is typically an interaction with mRNA. “Target oligonucleotide” relates to the oligonucleotides that are targeted by the antisense oligonucleotide or siRNAs.

In the context of the presently disclosed subject matter, “modulation” means either an increase (stimulation) or a decrease (inhibition) in the amount or levels. “Modulation of gene expression” means either an increase (stimulation) or a decrease (inhibition) in the amount or levels of a nucleic acid encoding the gene/protein of interest, e.g., DNA or RNA. Inhibition is often the preferred form of modulation of expression and mRNA is often a preferred target nucleic acid. The oligonucleotides, oligomeric compounds, compositions, reagents or methods of the presently disclosed subject matter are used to modulate the expression of a target nucleic acid. “Modulators” are those Oligomeric compounds that decrease or increase the expression of a nucleic acid molecule encoding a target and which comprise at least an nucleobase portion that is complementary to a preferred target segment.

For the delivery methods of the presently disclosed subject matter, all oligonucleotides or oligomeric compounds that have to be delivered into the cell can be used, as long as they fulfil the specifications as described herein. In a particular embodiment, the oligonucleotide or oligomeric compound that needs to be delivered to the cell targets a nucleic acid within the cell and thereby has a antisense sequence compared to that target nucleic acid in the cell.

The selection of oligonucleotides suitable for targeting a nucleic acid, namely selection of the antisense oligonucleotide sequence, is known in the art. It is known in the art that for the targeting of nucleic acids in order to modulate the expression, usually includes determination of at least one target region, segment, or site within the target nucleic acid for the interaction to occur such that the desired effect, e.g., modulation of expression, will result. Within the context of the presently disclosed subject matter, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the presently disclosed subject matter, are defined as positions within a target nucleic acid. The terms region, segment, and site can also be used to describe an oligonucleotide of the presently disclosed subject matter such as, for example, a gapped oligomeric compound having 3 separate segments.

The different regions, sites or segments which can efficiently be targeted are known in the art and are briefly exemplified herein. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-WG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the presently disclosed subject matter, “start codon” and “translation initiation codon” refer to the codon or codons that are used in vivo to initiate translation of an mRNA transcribed from a gene encoding a nucleic acid target, regardless of the sequence(s) of such codons.

It is also known in the art that a translation termination codon (or “stop codon”) of a gene may have one of three sequences, i.e., 5′-UM, 5′-UAG and 5′-UGA (the corresponding DNA sequences are 5′-TAA, 5′-TAG and 5′-TGA respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, as is known in the art, the “start codon region” (or “translation initiation codon region”) and the “stop codon region” (or “translation termination codon region”) are all regions which may be targeted effectively with antisense oligonucleotides.

The “open reading frame” (ORF) or “coding region,” which is known in the art to refer to the region between the translation initiation codon and the translation termination codon, is also a region which may be targeted effectively. Within the context of the presently disclosed subject matter, a suitable region is the intragenic region encompassing the translation initiation or termination codon or the open reading frame (QRF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene).

The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site and is also considered in the prior art as a good target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, seems also to be particularly useful in situations where aberrant splicing is implicated in disease, or where an overproduction of a particular splice product is implicated in disease. Aberrant fusion junctions due to rearrangements or deletions are also suitable target sites. mRNA transcripts produced via the process of splicing of two (or more) mRNAs from different gene sources are known as “fusion transcripts”. It is also known that introns can be effectively targeted using oligonucleotides targeted to, for example, pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequences. Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing: transcripts that terminate at unique polyA sites. Within the context of the presently disclosed subject matter, the types of variants described herein are also suitable target nucleic acids.

For identifying a good modulator of gene expression, namely identifying an oligonucleotide directed to a good target sequence, a screening assay can be used. The screening method comprises the steps of contacting a target segment of a nucleic acid molecule encoding a target with one or more candidate modulators, and selecting for one or more candidate modulators which decrease or increase the expression of a nucleic acid molecule encoding a target. Once it is shown that the candidate modulator or modulators are capable of modulating (e.g. either decreasing or increasing) the expression of a nucleic acid molecule encoding a target, the modulator may then be employed in further investigative studies of the function of a target, or for use as a research, diagnostic, or therapeutic agent in accordance with the presently disclosed subject matter.

Oligomeric compounds and oligonucleotides. In the context of the presently disclosed subject matter, the term “oligomeric compound” refers to a polymeric structure which is single or double stranded and which is linear or circular or of any other possible form (i.e. loop, etc.) capable of hybridizing a region of a nucleic acid molecule or expressing a nucleic acid molecule or protein. This term includes natural or modified oligonucleotides (oligonucleotide analogs, oligonucleotide mimetics) and combinations of these and includes double-stranded oligonucleotides such as siRNAs, hybridization complexes, hairpin oligonucleotides and the like and also include nucleic acid vectors.

As used herein, the term “oligonucleotide” refers to a single-stranded polymeric structure capable of hybridizing a region of a nucleic acid molecule and is constructed of monomeric nucleotide units, modified or natural/unmodified and thus refers to an oligomer or polymer of ribonucleic acid (RNA) or deoxyribonucleic acid (DNA) which can be modified. Oligonucleotides can be hybridized to form double-stranded oligomeric compounds that can be blunt ended or may include overhangs. In general, an oligonucleotide comprises a plurality of nucleotide residues where the residues are composed of different subunits, namely (naturally-occurring) nucleobases, sugars and covalent internucleoside linkages. For ribonucleic acid, the natural nucleotide residues are ribonucleotides (comprising a 2′-OH), while for deoxyribonucleic acid, the nucleotide residues are deoxyribonucleotides.

As is known in the art, a “nucleoside” is a base-sugar combination. The base portion of the nucleoside is normally a heterocyclic base or also called nucleobase moiety. The two most common classes of such heterocyclic bases are purines and pyrimidines. “Nucleotides” or “nucleotide residues” are nucleosides that further include a phosphate group covalently linked to the sugar portion of the nucleoside and which can be modified. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound like an oligonucleotide. The respective ends of this linear polymeric structure can be joined to form a circular structure by hybridization or by formation of a covalent bond, however, open linear structures are generally suitable. Within the oligonucleotide structure, the phosphate groups are commonly referred to as forming the internucleoside linkages of the oligonucleotide. The normal internucleoside linkage of RNA and DNA is a 3′ to 5′ phosphodiester linkage.

As used herein “DNA-oligonucleotide” refers to an oligonucleotide which is fully composed of natural deoxyribonucleotides.

As used herein “RNA-oligonucleotide” refers to an oligonucleotide which comprises at least 20% ribonucleotides or modified ribonucleotides, so which comprises nucleotide residues with a 2′-substituent.

In order to determine the percentage of nucleotides with a specific characteristic (i.e. natural or with modification, deoxyribonucleotides or ribonucleotides, etc.) the number of nucleotides with the specific chracteristic are counted and divided through the total number of nucleotides and multipplied by 100. For example for the oligonucleotide CCAUCCcgacctcgCGCUCC wherein the two ends are modified ribonucleotides and the bold nucleotides are deoxyribonucleotides, the percentage of modified nucleotides in this oligonucleotide is 60%.

As used herein, a “hybridization complex” refers to a duplex of two oligonucleotides (two oligonucleotides hybridized to each other) which are complementary to each other. The hybridization complex of the presently disclosed subject matter comprises (i) a first oligonucleotide strand, wherein the first oligonucleotide strand comprises a fully or partially modified oligonucleotide that hybridizes to at least a portion of a target nucleic acid (mRNA or DNA) and (ii) a second oligonucleotide strand, wherein the second oligonucleotide strand comprises a DNA-oligonucleotide that is, complementary, more in particular 100% complementary to at least a region of the first oligonucleotide strand. In accordance with this presently disclosed subject matter, a hybridization complex comprising the antisense strand oligonucleotide and its complement sense strand oligonucleotide can be designed for a specific target nucleic acid. The sense strand of the duplex is designed and synthesized as the complement of the antisense strand and may also contain modifications or additions to either terminus.

It is known in the prior art that oligomeric compounds, oligonucleotides or nucleotides can be modified (so that they become non-natural) and still have hybridization capacity or even have an increased hybridization capacity or other advantageous properties. Such non-naturally occurring oligonucleotides can be advantageous with respect to, for example, enhanced cellular uptake, enhanced affinity for nucleic acid target and increased stability in the presence of nucleases. In general, modifications of nucleotides can be divided in three groups, modifications (1) of the internucleoside linkages, so of the phosphate moiety, (2) of the nucleobase and (3) of the sugar subunit. The linkages joining the sugar and base subunits, the sugar moieties, and the heterocyclic base moieties can be independently or collectively modified giving rise to a plurality of motifs for the resulting oligomeric compounds including chimeras, hemimers and gapmers. Subsequently, the term “oligonucleotide analog” refers to oligonucleotides that have one or more non-naturally occurring portions which function in a similar manner to oligonulceotides.

In the context of this presently disclosed subject matter, the term “oligonucleoside” refers to nucleosides that are joined by internucleoside linkages that are unnatural so that for example have no phosphorus atoms and is a term used to designate modified oligonucleotides wherein the internucleotide linkage is modified.

Additionally, the oligomeric compounds are also considered as being modified when a conjugate moiety is conjugated thereto or other modifiecations are performed for example at the 3′-end or 5′-end of the oligonucleotide, such as the coupling of a linker to another oligonucleotide or conjugate moiety.

In addition to the internucleoside linkage modifications described above, the nucleosides of the oligomeric compounds of the presently disclosed subject matter can have a variety of other modifications so long as these other modifications either alone or in combination with other nucleosides enhance one or more of the desired properties described above. Thus, for nucleotides that are incorporated into oligonucleotides of the presently disclosed subject matter, these nucleotides can have sugar portions that correspond to naturally-occurring sugars or modified sugars. Representative modified sugars include carbocyclic or acyclic sugars, sugars having substituent groups at one or more of their 2′, 3′ or 4′ positions and sugars having substituents in place of one or more hydrogen atoms of the sugar. Additional nucleosides amenable to the presently disclosed subject matter having altered base moieties and or altered sugar moieties are disclosed in U.S. Pat. No. 3,687,808 and PCT application PCT/US89/02323.

Altered base moieties or altered sugar moieties also include other modifications consistent with the spirit of this presently disclosed subject matter. Such oligonucleotides are best described as being structurally distinguishable from, yet functionally interchangeable with, naturally occurring or synthetic wild type oligonucleotides. All such modifications are comprehended by this presently disclosed subject matter as long as they function effectively to mimic the structure of a desired RNA or DNA strand.

The oligomeric compounds in accordance with this presently disclosed subject matter can comprise from about 8 to about 80 nucleotides (i.e. from about 8 to about 80 linked nucleosides). One of ordinary skill in the art will appreciate that the presently disclosed subject matter embodies oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. In some embodiments, the oligomeric compounds of the presently disclosed subject matter are 12 to 50 nucleotides in length. One having ordinary skill in the art will appreciate that this embodies oligomeric compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In other embodiments, the oligomeric compounds of the presently disclosed subject matter are 15 to 30 nucleotides in length. One having ordinary skill in the art will appreciate that this embodies oligomeric compounds of 15, 16, 17, 18, 19, 20,21, 22, 23, 24, 25, 26, 27, 28, 29, or 30 nucleotides in length. In yet further embodiments, oligomeric compounds are oligonucleotides from about 8 to about 30 nucleotides, or from about 15 to about 30 nucleotides or from about 10 to about 25 or 20 nucleotides and every length in between.

Hybridization and complementarity. In the context of this presently disclosed subject matter, “hybridization” or “hybridizing” means the pairing of complementary strands of oligonucleotides. Pairing typically involves hydrogen bonding, which may be Watson-Crick, Hoogsteen or reversed Hoogsteen hydrogen bonding, between complementary nucleoside or nucleotide bases (nucleobases) of the strands of oligonucleotides. For example, adenine and thymine are complementary nucleobases that pair through the formation of hydrogen bonds. Hybridization can occur under varying circumstances.

An oligonucleotide of the presently disclosed subject matter is “specifically hybridizable” when binding of the compound to the target nucleic acid interferes with the normal function of the target nucleic acid to cause a loss of activity, and there is a sufficient degree of complementarily to avoid non-specific binding of the oligonucleotide to non-target nucleic acid sequences under conditions in which specific binding is desired, i.e., under physiological conditions in the case of in vivo assays or therapeutic treatment, and under conditions in which assays are performed in the case of in vitro assays.

In the presently disclosed subject matter the phrase “stringent hybridization conditions” or “stringent conditions” refers to conditions under which an oligonucleotide of the presently disclosed subject matter will hybridize to its target sequence, but to a minimal number of other sequences. Stringent conditions are sequence-dependent and will vary with different circumstances and in the context of this presently disclosed subject matter, “stringent conditions” under which oligonucleotides hybridize to a target sequence are determined by the nature and composition of the oligonucleotide and the assays in which they are being investigated. “Complementary” as used herein, refers to the capacity for precise pairing of two nucleobases regardless of where the two are located. For example, if a nucleobase at a certain position of an oligonucleotide is capable of hydrogen bonding with a nucleobase at a certain position of a target nucleic acid, then the position of hydrogen bonding between the oligonucleotide and the target nucleic acid is considered to be a complementary position. The oligonucleotide and the target nucleic acid are complementary to each other when a sufficient number of complementary positions in each molecule are occupied by nucleobases that can hydrogen bond with each other. Thus, “specifically hybridizable” and “complementary” are terms which are used to indicate a sufficient degree of precise pairing or complementarily over a sufficient number of nucleobases such that stable and specific binding occurs between the oligonucleotide and a target nucleic acid.

It is understood in the art that the sequence of the oligonucleotide need not be 100% complementary to that of its target nucleic acid to be specifically hybridizable. Moreover, an oligomeric compound may hybridize over one or more segments such that intervening or adjacent segments are not involved in the hybridization event (e.g., a loop structure or hairpin structure). In some embodiments, oligonucleotides of the presently disclosed subject matter comprise at least 70% sequence complementarily to a target region within the target nucleic acid, in further embodiments they comprise 90% sequence complementarily and in yet further embodiments they comprise 95% sequence complementarily to the target region within the target nucleic acid sequence to which they are targeted. For example, an oligonucleotide in which 18 of 20 nucleobases of the oligonucleotide are complementary to a target region, and would therefore specifically hybridize, would represent 90 percent complementarily. In this example, the remaining non-complementary nucleobases may be clustered or interspersed with complementary nucleobases and need not be contiguous to each other or to complementary nucleobases. As such, an oligonucleotide which is 18 nucleobases in length having 4 (four) noncomplementary nucleobases which are flanked by two regions of complete complementarily with the target nucleic acid would have 77.8% overall complementarily with the target nucleic acid and would thus fall within the scope of the presently disclosed subject matter. Percent complementarily of an oligonucleotide with a region of a target nucleic acid can be determined routinely using BLAST programs (basic local alignment search tools) and PowerBLAST programs known in the art (Altschul et al., J. Mol. Biol., 1990, 215, 403-410, Zhang and Madden, Genome Res., 1997, 7, 649-656).

Hybridization and the formation of duplexes or hybridization complexes of oligonucleotides is obtained by methods exemplified herein and involves methods known in te art.

General Oligomer and oligonucleotide synthesis. Synthesis of oligomeric compounds, oligonucleotides or oligomerization of modified and unmodified nucleosides or nucleotides can be performed according to literature procedures for oligonucleotides or nucleic acids in general, mostly using solid-phase phosphoramidite chemical synthesis. For DNA synthesis (Protocols for Oligonucleotides and Analogs, Ed. Agrawal (1993), Humana Press) and/or synthesis of RNA like compounds (Scaringe, Methods, 2001, 23, 206-217; Gallo et al., Tetrahedron, 2001, 57, 5707-5713) different procedures have to be used and will be adapted as appropriate. In addition specific protocols for the synthesis of oligomeric compounds used in the presently disclosed subject matter are illustrated in the examples below. Alternatively, RNA molecules may be generated by in vitro and in vivo transcription of DNA sequences encoding the antisense RNA molecule. The DNA sequences described herein may thus be used to prepare such molecules. Such DNA sequences may be incorporated into a wide variety of vectors which incorporate suitable RNA polymerase promoters such as the T7 or SP6 polymerase promoters. Alternatively, antisense cDNA constructs that synthesize anti-sense RNA constitutively or inducibly, depending on the promoter used, can be introduced stably into cell lines Oligonucleotides and modifications thereof can furthermore be synthesized by methods disclosed herein or known in the prior art or can be purchased from various synthesis companies such as for example Dharmacon Research Inc., (Lafayette, Colo.), Eurogentec, RNA-Tec (Leuven, Belgium), etc.

Conjugation of the sense- and/or antisense-oligonucleotide of the hybridization complex. In a particular embodiment, the oligomeric compounds, more in particular the oligonucleotides can be conjugated to a conjugate moiety in order to modify the properties of the oligomeric compound, yet oligonucleotide. In a particular embodiment, the second sense oligonucleotide is conjugated to a conjugate moiety, while also the first oligonucleotide of the hybridization complex can be conjugated. Such conjugate moiety coupled to the sense oligonucleotide of the hybridization complex can be selected from lipophilic molecules (aromatic and non-aromatic) including steroid molecules such as cholesterol; proteins (e.g., antibodies, enzymes, serum proteins); peptides such as Tat-peptide; vitamins (water- soluble or lipid-soluble); polymers (water-soluble or lipid-soluble); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonuclease/intercalators; crosslinking agents (e.g., photoactive, redox active), and combinations and derivatives thereof, as described herein below or in the prior art and can be performed for multiple reasons like the increase of the cellular uptake, the modulation of the duplex stability, etc.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic. Other embodiments encompass the reaction between a carboxylgroup or activated carboxylgroup and amines or substituted amines in order to create amides. For the creation of these conjugates, also linkers can be used.

Methods for conjugation of nucleic acids with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety. Also patent literature teaches the preparation thereof such as in U.S. Pat. Nos. 6,444,806; 6,486,308; 6,525,031; 6,528,631; 6,559,279; each of which is herein incorporated by reference.

Modifications of oligomeric compounds—oligonucleotides. As mentioned earlier, oligomeric compounds and oligonucleotides in particular can be modified in order to obtain advantageous properties. Modifications can be performed of the internucleoside linkage, of the sugar subunit or of the nucleobase and all combinations thereof. Multiple different modifications may be present in one nucleotide, oligonucleotide or oligomeric compound. Some examples of modifications will be described hereunder, but this is a non-limitative description of all possibilities which are described in literature. Representative patents or articles that teach the preparation of the modifications can easily be found by a person skilled in the art through patent databases and journal websites or publications and will therefore not all me mentioned herein, but are considered as being state of the art and therefore can be incorporated herein by reference.

For the current presently disclosed subject matter, such modifications are important since they are necessary to increase the stability of the first antisense oligonucleotide strand of the hybridization complex. As mentioned before, the antisense strand of the hybridization complex is modified and can comprise multiple different modifications, whether of the internucleoside linkage, the sugar subunit or the base or combiantions thereof and can be selected fro mmodifications known in the art or which will be used in the future.

Non-natural internucleotide linkages. As defined in this specification, oligomeric compounds or oligonucleotides having modified internucleoside linkages include internucleoside linkages that retain a phosphorus atom and internucleoside linkages that do not have a phosphorus atom, which can also be considered as oligomeric compounds or oligonucleotides.

An exemplary phosphorus-containing modified internucleoside linkage is the phosphorothioate internucleoside linkage and other examples include chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones, sulfide, sulfoxide and sulfone backbones; formacetal and thioformacetal backbones; methylene formacetal and thioformacetal backbones; riboacetal backbones; alkene containing backbones; sulfamate backbones; methyleneimino and methylenehydrazino backbones; sulfonate and sulfonamide backbones; amide backbones; and others having mixed N. O. S and CH2 component parts. More specific examples include heteroatom internucleoside linkages, in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂—[wherein the native phosphodiester internucleotide linkage is represented as —O—P(═O)(OH)—O—CH₂—].

As an example, hereunder is the structure of a phosphorothioate internucleoside link as can be found in the prior art.

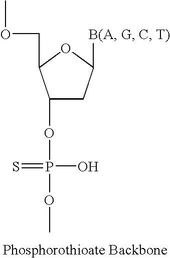

Modifications of the sugar subunit or combinations of modifications of the internucleotide linkage and the sugar subunit. In nucleotides or oligonucleotides only the furanose ring (sugar surrogate) or both the furanose ring and the internucleotide linkage can be replaced with novel groups. The furanose ring modifications comprise substitutions of the furanose ring or complete chanegemnt of the sugar ring. The heterocyclic base moiety or a modified heterocyclic base moiety is maintained for hybridization with an appropriate target nucleic acid.

Oligomeric compounds used in the method of the presently disclosed subject matter may contain one or more substituted sugar moieties. Many sugar substituent groups are known in the art (i.e. alkyl; alkenyl; alkynyl; alkylaryl; OH; SH; halogen such as F, Cl, Br; CN; CF₃; O—, S—, or N-alkyl; O—, S—, or N-alkenyl; O—, S— or N-alkynyl; O-alkyl-O-alkyl, lower alkyl, substituted lower alkyl, alkenyl, alkynyl, alkaryl, aralkyl, O-alkaryl or O-aralkyl, SH, SCH₃, OCN, OCF₃, SOCH₃, SO₂CH₃, ONO₂, NO₂, N₃, NH₂, heterocycloalkyl, heterocycloalkaryl, aminoalkylamino, polyalkylamino, substituted silyl, etc.).

An example modification includes 2′-methoxyethoxy (2′-O—CH₂CH₂OCH₃), also known as 2′-O-(2-methoxyethyl) or 2′-MOE (Martin et al., Helv. Chim. Acta, 1995, 78, 486-504) i.e., an alkoxyalkoxy group. A further example modification includes 2′-dimethylaminooxyethoxy, i.e., a 2′-O—(CH₂)₂—O—N(CH₃)₂ group, also known as 2′-DMAOE and 2′-dimethylaminoethoxyethoxy (also known in the art as 2′-O-dimethyl-aminoethoxy ethyl or 2′-DMAEOE), i.e., 2′-O—(CH₂)₂—O—(CH₂)₂—N(CH₃)₂. Other sugar substituent groups include halogen atoms such as fluoro (F), chloro (Cl) or bromo (Br); methoxy (-O-CH3), ethoxy, propyloxy (or propoxy), aminopropoxy (—OCH₂CH₂CH₂NH₂), allyl (—CH₂—CH═CH₂) and —O-allyl (—O—CH₂—CH═CH₂). Example sugar substituent groups include O[(CH2)ₙO]ₘCH₃, O(CH₂)ₙOCH3, O(CH₂)ₙNH₂, O(CH₂)ₙCH3 O(CH2)ₙONH₂ and O(CH₂)ₙON[(CH₂)ₙCH₃)]₂, where n and m are from 0 or 1 to about 10. 2′-sugar substituent groups may be in the arabino (up) position or ribo (down) position. An example 2′-arabino modification is 2′-F.

Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide.

In a particular embodiment of the presently disclosed subject matter, the first oligonucleotide strand of the hybridization complex comprises a 2′ modification, which is in particular selected from the group consisting of 2′-halo, 2′-O-methyl, 2′-O-ethyl, 2′-O-propyl, 2′-O-butyl; 2′-O-pentyl, 2′-O-(2-methoxyethyl), and 2′-O-[2-[N,N-dimethylamino-oxy]-ethyl], or more in particular, is a 2′-O-methyl group.

Other examples of sugar modifications are described below. An oligonucleotide mimetic that has been shown to have excellent hybridization properties, is referred to as a peptide nucleic acid (PNA). In PNA oligomeric compounds, the sugar-backbone of an oligonucleotide is replaced with an amide containing backbone, in particular an aminoethylglycine backbone (for further description see for example Nielsen et al., Science, 1991, 254, 1497-1500 and several patents have been filed hereon). PNA has been modified to incorporate numerous modifications since the basic PNA structure was first prepared and these modifications can also be applied.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. A class of linking groups have been selected to give a non-ionic modification. The non-ionic morpholino-based oligomeric compounds seem to be less likely to have undesired interactions with cellular proteins (Dwaine A. Braasch and David R. Corey, Biochemistry, 2002, 41(14), 4503-4510).

A further class of oligonucleotide mimetic is referred to as cyclohexenyl nucleic acids (CeNA). The furanose ring normally present in an DNA/RNA molecule is replaced with a cyclohexenyl ring. Fully modified CeNA oligomeric compounds and oligonucleotides having specific positions modified with CeNA have been prepared and studied (see Wang et al., J; Am. Chem. Soc., 2000, 122, 8595-8602). In general the incorporation of CeNA monomers into a DNA chain increases its stability of a DNA/RNA hybrid. CeNA oligoadenylate formed complexes with RNA and DNA complements with similar stability to the native complexes. The incorporation of CeNA into a sequence targeting RNA was stable to serum and able to activate E. Coli RNase resulting in cleavage of the target RNA strand.

Another class of oligonucleotide mimetic (anhydrohexitol nucleic acid) can be prepared from one or more anhydrohexitol nucleosides as described in Wouters and Herdewijn, Bioorg. Med. Chem. Lett., 1999, 9, 1563-1566.

A further example modification includes Locked Nucleic Acids (LNAs) in which the 2′- hydroxyl group is linked to the 4′ carbon atom of the sugar ring thereby forming a 2′-C, 4′-C-oxymethylene linkage thereby forming a bicyclic sugar moiety. LNA has been shown to form exceedingly stable LNA:LNA duplexes (Kosdkin et al., J. Am. Chem. Soc., 1998, 120, 13252-13253). Introduction of 3 LNA monomers (T or A) significantly increased melting points (Tm=+15/+11) toward DNA complements. Novel modifications of LNAs are also being developed.

It has been demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

Subsequently, in another particular embodiment, the 2′ modification comprises a locked nucleic acid or locked nucleotide(s), wherein the locked nucleic acid is characterized by a methylene bridge that connects a 2′-oxygen with a 4′-carbon of a ribose.

Many other modifications have been described, including but certainly not limited to oligonucleotides to incude bicyclic and tricyclic nucleoside analogs, to include a cyclobutyl moiety instead of the furanosyl ring, and many others.

Modified Nucleobases/Naturally occurring nucleobases. Oligomeric compounds may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as: 5-methylcytosine (5-me-C), 5-hydroxymethyl cytosine, xanthine, hypoxanthine, 2-amino-adenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl-uracil and cytosine and other alkynyl derivatives of pyrimidine bases; 6-aza uracil, cytosine and thymine; 4-thiouracil; 8-halo, 8-amino, 8-thiol, 8-thioalkyl, 8-hydroxyl and other 8-substituted adenines and guanines; 5-halo particularly 5-bromo, 5- trifluoromethyl and other 5-substituted uracils and cytosines, 7-methylguanine and 7-methyladenine, 2-F-adenine, 2-amino-adenine, 8-azaguanine and 8-aza-adenine, 7-deaza-guanine and 7-deaza-adenine and 3-deaza-guanine and 3-deaza-adenine.

Heterocyclic base moieties may also include those in which the purine or pyrimidine base is replaced with other heterocycles, for example 7-deaza-adenine, 7-deazaguanosine, 2-aminopyridine and 2-pyridone. Certain of these nucleobases are particularly useful for increasing the binding affinity of the oligomeric compounds. These include 5-substituted pyrimidines, 6-azapyrimidines and N-2, N-6 and O-6 substituted purines, including 2-aminopropyladenine, 5-propynyluracil, 5-propynylcytosine and 5-methylcytosine and are exemplery base substitutions, such as when combined with 2′-O-methoxyethyl sugar modifications. Also polycyclic heterocyclic compounds in place of one or more heterocyclic base moieties have been reported, such as tricyclic heterocyclic compounds. These compounds are routinely used in antisense applications to increase the binding properties of the modified strand to a target strand. A class of representative base modifications include tricyclic cytosine analog, termed “G clamp” (Lin, et al., J. Am. Chem. Soc. 1998, 120, 8531). This analog makes four hydrogen bonds to a complementary guanine (G) within a helix by simultaneously recognizing the Watson-Crick and Hoogsteen faces of the targeted G.

Representative cytosine analogs that make 3 hydrogen bonds with a guanosine in a second strand include 1,3- diazaphenoxazine-2-one, 1,3 diazaphenothiazine-2-one, and 6,7,8,9-tetrafluoro-1,3-diazaphenoxazine-2-one. Incorporated into oligonucleotides these base modifications were shown to hybridize with complementary guanine and the latter was also shown to hybridize with adenine and to enhance helical thermal stability by extended stacking interactions.

3′-endo modifications. Another type of modification is directed to the induction of a 3′-endo sugar conformation. A nucleoside can incorporate synthetic modifications of the heterocyclic base, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement (e.g. trigger) of RNA interference, which is supported in part by the fact that duplexes composed of 2′ deoxy-2′-F-nucleosides appears efficient in triggering RNAi response in the C. elegans system. Properties that are enhanced by using more stable 3′-endo nucleosides include but aren't limited to modulation of pharmacokinetic properties through modification of protein binding, protein off-rate, absorption and clearance, modulation of nuclease stability as well as chemical stability; modulation of the binding affinity and specificity of the oligomer (affinity and specificity for enzymes as well as for complementary sequences); and increasing efficacy of RNA cleavage. One routinely used method of modifying the sugar puckering is the substitution of the sugar at the 2′-position with a substituent group that influences the sugar geometry. The influence on ring conformation is dependent on the nature of the substituent at the 2′-position, i.e. 2′-F and 2′-OH yield different duplex stabilities.

Chimeric oligomeric compounds. It is not necessary for all positions in an oligomeric compound or oligonucleotide to be uniformly modified, and in fact more than one of the aforementioned modifications may be incorporated in a single oligomeric compound or even at a single monomeric subunit such as a nucleoside within a oligomeric compound or oligonucleotide. In a particular emboidment, the presently disclosed subject matter uses oligomeric compounds which are chimeric oligomeric compounds as antisnese first strands. “Chimeric” oligomeric compounds or oligonucleotides or “chimeras,” in the context of this presently disclosed subject matter, are oligomeric compounds that contain two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. In a particuar embodment of the presently disclosed subject matter, the first oligonucleotide strand of the hybridization complex comprises at least one region modified so as to confer increased resistance to nuclease degradation. An additional region of the oligomeric compound may serve as a substrate for enzymes capable of cleaving RNA:DNA or RNA:RNA hybrids, such as RNase H, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the presently disclosed subject matter may be formed as composite structures of two or more oligonucleotides or oligonucleotide analogs, oligonucleosides and/or oligonucleotide mimetics as described above. Such oligomeric compounds have also been referred to in the art as hybrids hemimers, gapmers or inverted gapmers. Representative United States patents that teach the preparation of such hybrid structures include, but are not limited to U.S. Pat. Nos. 5,013,830; 5,149,797; 5,220,007; 5,256,775; 5,366,878; 5,403,711; 5,491,133; 5,565,350; 5,623,065; 5,652,355, 5,652,356; and 5,700,922, each of which is herein incorporated by reference in its entirety.

It is the purpose of the presently disclosed subject matter to use hybridization complexes wherein the first and second oligonucleotide are hybridized in a strong enough way so to yield a strong enough duplex stability to obtain an interaction during the delivery phase, but allowing for the degradation of the second oligonucleotide strand and not of the first strand. Therefore, modified oligonucleotides or chimeric structures can be used and selected that increase the duplex stability, but allow for the degradation of the second oligonucleotide, but not of the first strand. On the other hand, a very tight binding is required for the second oligonucleotide strand to its target nucleic acid, i.e. mRNA.

DNA:RNA hybrid duplexes, are usually less stable than pure RNA:RNA duplexes, and depending on their sequence may be either more or less stable than DNA:DNA duplexes (Searle et al., Nucleic Acids Res., 1993, 21, 2051 2056). The structure of a hybrid duplex is intermediate between A- and B-form geometries, which may result in poor stacking interactions (Lane et al., Ear. J: Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic oligonucleotide strand to an RNA target strand. In the case of antisense, effective inhibition of the mRNA requires that the antisense DNA have a very high binding I affinity with the mRNA. Otherwise the desired interaction between the synthetic oligonucleotide strand and target mRNA strand will occur infrequently, resulting in decreased efficacy.

Dendrimeric bioconiugate delivery. The presently disclosed subject matter furthermore relates to dendrimeric bioconjugates (also called bioconjugate dendrimers throghout the text) wherein the bioconjugate dendrimer comprises a conjugate moiety coupled to a dendrimeric structure and to their use to deliver hybridization complexes, oligonucleotides or duplexes, as described herein, to cells for modulation of gene expression (i.e. antisense or antigene therapy/research, RNA interference for therapy and research purposes). Thus, the presently disclosed subject matter also relates to the delivery of oligonucleotides for antisense applications, ribozymes, oligonucleotides for RNA interference and for antigene applications.

This dendrimeric bioconjugate can be used for the delivery of oligonucleotides to cells for antisense or antigene therapy/research and thus the presently disclosed subject matter relates to the use of the dendrimeric bioconjugate for the in vitro or in vivo inhibition of transcription or translation of a gene. The presently disclosed subject matter relates also to the use of the dendrimeric bioconjugates as a medicine and for the manufacture of a medicament for the treatment or prevention of a specific disease. The presently disclosed subject matter also relates to the process for preparing the dendrimeric bioconjugates.

The presently disclosed subject matter provides thus for dendrimeric bioconjugates and compositions or reagents containing the same wherein the dendrimeric bioconjugate includes one or more conjugate moieties covalently linked to a dendrimeric structure, wherein the dendrimeric structure non-covalently interacts (i.e. hybridizes, ionic interaction, etc.) with oligomeric compounds. The dendrimeric structure of the presently disclosed subject matter can be covalently attached, optionally through one or more linkers, to one or more conjugate moieties. The dendrimeric bioconjugate ensures modified or enhanced pharmacokinetic, pharamcodynamic, and other properties for the oligomeric compounds compared with the oligomeric compounds alone. A dendrimeric bioconjugate that can modify the pharmacokinetic or pharmacodynamic properties of an oligomeric compound, can modify or improve activity, resistance to degradation, sequence-specific hybridization, cellular distribution, bioavailability, metabolism, excretion, permeability and/or cellular uptake of the oligomeric compound. The dendrimeric bioconjugate of the presently disclosed subject matter particularly modifies the cellular uptake of the oligomeric compound. The dendrimeric bionconjugate of the presently disclosed subject matter combines a dendrimeric structure with a conjugate moiety which is known fror its cellular penetration enhancement properties.

A dendrimeric structure is defined as having repetitive and branched units, wherein the units are molecularly quite uniform. The amount of units in the dendrimeric structure is at least 2, but can be 3, 4, 5, 6, 7, 8, 9, 10 or much more. Representatvie dendrimeric structures can be cationic polymers such as starburst polyamidoamine (PAMAM), or a variety of other polymers such as, for example, polyester dendrimers (Gillies E R, Dy E, Frechet J M, Szoka F C. Mol Pharm. March-April 2005; 2(2):129-38) or can comprise multiple, but at least two, oligonucleotides covalently linked to each other.

In a particular embodiment, the presently disclosed subject matter relates to dendrimeric bioconjugates, wherein the dendrimeric bioconjugate comprises a conjugate moiety and a dendrimeric oligonucleotide structure. A dendrimeric oligonucleotide structure comprises multiple (more than one) oligonucleotides covalently coupled to each other with or wothout linking moieties and all the oligonucleotides are able to hybridize with oligomeric compounds that can than be delivered to the cell. The oligonucleotides of the dendrimeric oligonucleotide structure can be identical in sequence or can be diffeernt. The dendrimeric oligonucleotide structure can contain 2, 3, 4, 8, or more oligonucleotides which are all covalently linked to each other through the use of linking moieties. The dendrimeric bioconjugate can furthermore also comprise a linker between the conjugate moiety and the dendrimeric oligonucleotide structure.

Representative conjugate moieties for the dendrimeric bioconjugate are lipophilic molecules (aromatic and non-aromatic) including steroid molecules such as cholesterol; peptides such as Tat-peptide or a variety of other cationic , amphipathic or hydrophobic peptides that have been used to enhance intracellular delivery(for an overview see Juliano R L. Peptide-oligonucleotide conjugates for the delivery of antisense and siRNA. Curr Opin Mol Ther. April 2005; 7(2):132-6); vitamins (water- soluble or lipid-soluble); polymers (water-soluble or lipid-soluble); small molecules including drugs, toxins, reporter molecules, and receptor ligands; carbohydrate complexes; The conjugate moieties for the dendrimeric bioconjugate are in a particular embodiment restricted to conjugate moieties which have an effect on the cellular uptake, and not on stability, duplex formation, etc. and thereby coprise lipofilic molecules such as cholesterol or fatty acids or peptides.

Conjugate moieties. In the constructs of the presently disclosed subject matter or the methods used, conjugate moieties are conjugated to oligomeric compounds or dendrimeric structures. One or more conjugate moieties can be conjugated to one oligomeric compound or dendrimeric structure or multiple conjugtae moieties can be linked or coupled to each other and to oligomeric compounds or dendrimeric structures.

Numerous conjugate moieties and their preparation are known in the art, for example, in WO 93/07883 and U.S. Pat. No. 6,395,492, each of which is incorporated herein by reference in its entirety. Oligonucleotide conjugates and their syntheses are also reported in comprehensive reviews by Manoharan in Antisense Drug Technology, Principles, Strategies, and Applications, S. T. Crooke, ea., Ch. 16, Marcel Dekker, Inc., 2001 and Manoharan, Antisense & Nucleic Acid Drug Development, 2002, 12, 103, each of which is incorporated herein by reference in its entirety. Hereunder, an overview of conjugate moieties known in the art for giving specific properties to the structures they are coupled to is given. As described herein above, a different selection of these conjugate moieties can be used for the conjugation of the sense strand of the hybridization complex than for the conjugate moieties of the dendrimeric bioconjugate.

Lipophilic conjugate moieties have already been used, for example, to counter the hydrophilic nature of an oligomeric compound and enhance cellular penetration. Lipophilic moieties include, for example, steroids and related compounds such as cholesterol (U.S. Pat. No. 4,958,013 and Letsinger et al., Proc. Natl. Acad. Sci. USA, 1989, 86, 6553), thiocholesterol (Oberhauser et al., Nucl. Acids Res., 1992, 20, 533), lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, 17-hydroxycorticosterone, their derivatives, and the like and also fatty acids and alanogues such as sfingolipids, arachidonic acid, oleic acid and the like.

Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include undecyl, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O. S. or N (e.g. geranyloxyhexyl). Further suitable lipophilic conjugate moieties include aliphatic derivatives of glycerols such as alkylglycerols, bis(alkyl) glycerols, tris(alkyl)glycerols, monoglycerides, diglycerides, and triglycerides. In some embodiments, the lipophilic conjugate is di-hexyldecyl-rac-glycerol or 1,2-di-O-hexyldecyl-rac-glycerol (Manoharan et al., Tetrahedron Lett., 1995, 36, 3651; Shea, et al., Nuc. Acids Res., 1990, 1S, 3777) or phosphonates thereof. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosenoic acids and the like.

In further embodiments, lipophilic conjugate moieties can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like.

Other suitable lipophilic conjugate moieties include menthols, trityls (e.g., dimethoxytrityl (DMT)), phenoxazines, lipoic acid, phospholipids, ethers, thioethers (e.g., hexyl-S-tritylthiol), derivatives thereof and the like. Preparation of lipophilic conjugates of oligomeric compounds are well-described in the art, such as in, for example, Saison-Behmoaras et al., EMBO], 1991, 10, 1111; Kabanov et al., FEBS Lett., 1990, 259, 327; Svinarchuk et al., Biochimie, 1993, 75, 49; (Mishra et al., Biochim. Biophys. Acta, 1995, 1264, 229, and Manoharan et al., Tetrahedron Lett., 1995, 36, 3651.

Conjugate moieties can also include vitamins. Vitamins are known to be transported into cells by numerous cellular transport systems. Typically, vitamins can be classified as water soluble or lipid soluble. Water soluble vitamins include thiamine, riboflavin, nicotinic acid or niacin, the vitamin B6 pyridoxal group, pantothenic acid, biotin, folic acid, the B2 cobamide coenzymes, inositol, choline and ascorbic acid. Lipid soluble vitamins include the vitamin A family, vitamin D, the vitamin E tocopherol family and vitamin K (and phytols). Related compounds include retinoid derivatives such as tazarotene and etretinate.

In some embodiments, the conjugate moiety includes folic acid (folate) and/or one or more of its various forms, such as dihydrofolic acid, tetrahydrofolic acid, folinic acid, pteropolyglutamic acid, dihydrofolates, tetrahydrofolates, tetrahydropterins, 1-deaza, 3-deaza, 5-deaza, 8-deaza, 10-deaza, 1,5-dideaza, 5,10-dideaza, 8,10-dideaza and 5,8-dideaza folate analogs, and antifolates. Folate is involved in the biosynthesis of nucleic acids and therefore impacts the survival and proliferation of cells. Folate cofactors play a role in the one-carbon transfers that are needed for the biosynthesis of pyrimidine nucleosides. Cells therefore have a system of transporting folates into the cytoplasm. Folate receptors also tend to be overexpressed in many human cancer cells, and folate-mediated targeting of oligonucleotides to ovarian cancer cells has been reported (Li, et al., Pharm. Res. 1998, 15, 1540). Preparation of folic acid conjugates of nucleic acids are described in, for example, U.S. Pat. No. 6,528,631, which is incorporated herein by reference in its entirety and similar synthetic procedures can be applied to conjugate the folic acid to dendrimeric structures.

Vitamin conjugate moieties include, for example, vitamin A (retinal) and/or related compounds. The vitamin A family (retinoids), including retinoic acid and retinal, are typically absorbed and transported to target tissues through their interaction with specific proteins such as cytosol retinol-binding protein type II (CRBP-II), retinol-binding protein (RBP), and cellular retinol-binding protein (CRBP). The vitamin A family of compounds can be attached to dendrimeric structures via acid or alcohol functionalities found in the various family members.

Alpha-Tocopherol (vitamin E) and the other tocopherols (beta through zeta) can be conjugated to dendrimeric structures to enhance uptake because of their lipophilic character. Also, vitamin D, and its ergosterol precursors, can be conjugated to dendrimeric structures through for example first their hydroxyl groups to carboxylgroups. Conjugation can then be effected directly to the dendrimeric structure or via a linker. Other vitamins that can be conjugated to dendrimeric structures in a similar manner include thiamine, riboflavin, pyridoxine, pyridoxamine, pyridoxal, deoxypyridoxine. Lipid soluble vitamin K's and related quinone-containing compounds can be conjugated via carbonyl groups on the quinone ring. The phytol moiety of vitamin K can also serve to enhance binding of the oligomeric compounds to cells.

Pyridoxal (vitamin B6) has specific B6-binding proteins. The role of these proteins in pyridoxal transport has been studied by Zhang et al., Proc. Natl Acad. Sci. USA, 1991, SS, 10407. Other pyridoxal family members include pyridoxine, pyridoxamine, pyridoxal phosphate, and pyridoxic acid.

Pyridoxic acid, niacin, pantothenic acid, biotin, folio acid and ascorbic acid can also be conjugated to dendrimeric structures.

Vitamin conjugate moieties can also be used to facilitate the targeting of specific cells or tissues. For example, vitamin D and analogs thereof can assist in transporting conjugated oligomeric compounds to keratinocytes, dermal fibroblasts, and other cells containing vitamin D3 nuclear receptors. Additionally, Vitamin A and other retinoids can be used to target cells with retinoid X receptors. Accordingly, vitamin-containing conjugate moieties can be useful in treating, for example, skin disorders such as psoriasis.

Conjugate moieties can also include polymers. Polymers can provide added bulk and various functional groups to affect permeation, cellular transport, and localization of the conjugated dendrimeric structure. In some embodiments, the conjugate polymer moiety has, for example, a molecular weight of less than about 40, less than about 30, or less than about 20 kDa. Additionally, polymer conjugate moieties can be water-soluble and optionally further comprise other conjugate moieties such as peptides, carbohydrates, drugs, reporter groups, or further conjugate moieties.

In some embodiments, polymer conjugates include polyethylene glycol (PEG) and copolymers and derivatives thereof. Conjugation to PEG has been shown to increase nuclease stability of an oligomeric compound. PEG conjugate moieties can be of any molecular weight including for example, about 100, about 500, about 1000, about 2000, about 5000, about 10,000 and higher. In some embodiments, the PEG conjugate moieties contains at least 4, at least 5, at least 6, at least 7, at least 8, at least 9, at least 10, at least 15, at least 20, or at least 25 ethylene glycol residues. In further embodiments, the PEG conjugate moiety contains from about 4 to about 10, about 4 to about 8, about 5 to about 7, or about 6 ethylene glycol residues. The PEG conjugate moiety can also be modified such that a terminal hydroxyl is replaced by alkoxy, carboxy, acyl, amide, or other functionality. Copolymers of PEG are also suitable as conjugate moieties. Preparation and biological activity of polyethylene glycol conjugates of oligonucleotides are described, for example, in Bonora, et al., Nucleosides Nucleotides, 1999, 18, 1723; Bonora, et al., Farmaco, 1998, 53, 634; Efimov, Bioorg. Whim. 1993, 19, 800; and Jaschke, et al., Nucleic Acids Res., 1994, 22, 4810. Further example PEG conjugate moieties and preparation is described in, for example, U.S. Pat. Nos. 4,904,582 and 5,672,662, each of which is incorporated by reference herein in its entirety.

Other polymers suitable as conjugate moieties include polyamines, polypeptides, polymethacrylates (e.g., hydroxylpropyl methacrylate (HPMA)) , poly(L-lactide), poly(DL lactide-co-glycolide (PGLA), polyacrylic acids, polyethylenimines (PEI), polyalkylacrylic acids, polyurethanes, polyacrylamides, N-alkylacrylamides, polyspermine (PSP), polyethers, cyclodextrins, derivatives thereof and co-polymers thereof. Many polymers, such as PEG and polyamines have receptors present in certain cells, thereby facilitating cellular uptake. Polyamines and other amine-containing polymers can exist in protonated form at physiological pH, effectively countering an anionic backbone of some oligomeric compounds, effectively enhancing cellular permeation. Some example polyamines include polypeptides (e.g., polylysine, polyornithine, polyhistadine, polyarginine, and copolymers thereof), triethylenetetraamine, spermine, polyspermine, spermidine, synnorspermidine, C-branched spermidine, and derivatives thereof. Preparation and biological activity of polyamine conjugates are described, for example, in Guzaev, et al., Bioorg. Med. Chem. Lett., 1998, 8, 3671; Corey, et al., J; Am. Chem. Soc., 1995, 117, 9373; and Prakash, et al., Bioorg. Med. Chem. Lett. 1994, 4, 1733.

Exemplary polypeptide conjugates are provided in, for example, Wei, et al., Nucleic Acids Res., 1996, 24, 655 and Zhu, et al., Antisense Res. Dev., 1993, 3, 265. Dendrimeric polymers can also be used as conjugate moieties, such as described in U.S. Pat. No. 5,714,166, which is incorporated herein by reference in its entirety.

As discussed above for polyamines and related polymers, other amine-containing moieties can also serve as suitable conjugate moieties due to, for example, the formation of cationic species at physiological conditions. Example amine-containing moieties include 3-aminopropyl, 3-(N,N-dimethylamino)propyl, 2-(2-(N,N-dimethylamino)ethoxy) ethyl, 2-(N-(2-aminoethyl)-N-methylaminooxy)ethyl, 2-(1-imidazolyl)ethyl, and the like. The G-clamp moiety can also serve as an amine-containing conjugate moiety (Lin, et al. , J. Am. Chem. Soc., 1998, 120, 8531). PAMAM is an example of a cationic dendrimeric polymer.

Conjugate moieties can also include peptides. Suitable peptides can have from 2 to about 30, 2 to about 20, 2 to about 15, or 2 to about 10 amino acid residues. Amino acid residues can be naturally or non-naturally occurring, including both D and L isomers. In some embodiments, peptide conjugate moieties are pH sensitive peptides such as fusogenic peptides. Fusogenic peptides can facilitate endosomal release of agents such to the cytoplasm. It is believed that fusogenic peptides change conformation in acidic pH, effectively destabilizing the endosomal membrane thereby enhancing cytoplasmic delivery of endosomal contents. Example fusogenic peptides include peptides derived from polymyxin B, influenza HA2, GALA, KALA, EALA, melittin-derived peptide, a-helical peptide or Alzheimer p-amyloid peptide, and the like. Preparation and biological activity of fusogenic peptide conjugates are described in, for example, Bongartz, et al., Nucleic Acids Res., 1994, 22, 4681 and U.S. Pat. Nos. 6,559,279 and 6,344,436.

Other peptides that can serve as conjugate moieties include delivery peptides which have the ability to transport relatively large, polar molecules (including peptides, oligonucleotides, and proteins) across cell membranes. Example delivery peptides include Tat peptide from HIV Tat protein and Ant peptide from Drosophila antenna protein. Conjugation of Tat and Ant with oligonucleotides is described in, for example, Astriab-Fisher, et al., Biochem. Pharmacol., 2000, 60, 83 and conjugation with dendrimeric structures is further exemplified herein. These and other delivery peptides that can be used as conjugate moieties are provided below in Table 1.

TABLE 1

| | | |
|---|---|---|
| SEQ | Delivery | |
| ID | Peptide Sequence | Source NO: |

| | | |
|---|---|---|
| 1 | RQIKIWFQNRRMKWKK | Antennapodia helix |
| | | 3 Antp-HD |
| 2 | GRKKRRQRRRPPQ | HIV Tat fragment |
| 3 | GWTLNSAGYLLGPINLKALAALAKKIL | Transportan: |
| | | chimeric galanin |
| | | and mastoporan |
| 4 | DAATATRGRSAASRPTERPRAPARSAS | HSV VP22 |
| | RPRRPVE | |
| 5 | KLALKLALKALKAALKLA | Amphiphilic peptide |
| | | 6 |
| 6 | GALFLGWLGAAGSTMGAWSQPKKKRKV | Signal sequence |
| | | based peptide I |
| 7 | AAVALLPAVLLALLAP | Signal sequence |
| | | based peptide II |
| 8 | PKKKRKV | SV40 antigen T |
| | | nuclear |
| | | localization |
| | | signal |
| 9 | MLFY | Platelet activating |
| | | factor receptor |
| | | of neutrophils |
| 10 | PQRRNRSRRRRFRGQ | FXR2P |
| 11 | IMRRRGL | angiogenin L |
| 12 | LQLPPLERLTL | HIV-1 Rev |
| 13 | ELALKLAGLDI | PKI- oc |
| 14 | DLQKKLEELEL | MAPKK |
| 15 | ALPHAIMRLDLA | actin I |
| 16 | PKKKRKV | simian virus 40 |
| | | large tumor |
| | | antigen |
| 17 | ALWKTLLKKVLKA | dermaseptin |

As used throughout, “peptide” includes not only the specific molecule or sequence recited herein (if present), but also includes fragments thereof and molecules comprising all or part of the recited sequence, where desired functionality is retained. In some embodiments, peptide fragments contain no fewer than 6 amino acids. Peptides can also contain conservative amino acid substitutions that do not substantially change its functional characteristics. Conservative substitution can be made among the following sets of functionally similar amino acids: neutral-weakly hydrophobic (A, G, P, S, T), hydrophilic-acid amine (N, D, Q, E), hydrophilic-basic (I, M, L, V), and hydrophobic-aromatic (F, W, Y). Peptides also include homologous peptides. Homology can be measured according to percent identify using, for example, the BLAST algorithm (default parameters for short sequences). For example, homologous peptides can have greater than 50, 60, 70, 80, 90, 95, or 99 percent identity. Methods for conjugating peptides to oligonucleotides is described in, for example, U.S. Pat. No. 6,559,279, which is incorporated herein by reference in its entirety.

Peptides can be used to increase the cellular uptake of conjugate, but conjugated delivery peptides can also help control localization of oligomeric compounds to specific regions of a cell, including, for example, the cytoplasm, nucleus, nucleolus, and endoplasmic reticulum (ER). Nuclear localization can be effected by conjugation of a nuclear localization signal (NLS). In contrast, cytoplasmic localization can be facilitated by conjugation of a nuclear export signal (NES). Peptides suitable for localization of conjugated dendrimeric structures in the nucleus include, for example, N,N-dipalmitylglycyl-apo E peptide or N,N- dipalmitylglycyl-apolipoprotein E peptide (dpGapoE) (Liu, et al., Arterioscler. Thromb. Vase. Biol., 1999, 19, 2207; Chaloin, et al., Biochem. Biophys. Res. Commun., 1998, 243, 601). Nucleus or nucleolar localization can also be facilitated by peptides having arginine and/or lysine rich motifs, such as in HIV-1 Tat, FXR2P, and angiogenin derived peptides (Lixin, et al., Biochem. Biophys. Res. Commun., 2001, 284, 185). Additionally, the nuclear localization signal (NLS) peptide derived from SV40 antigen T (Brander, et al., Nature Biotech, 1999, 17, 784) can be used to deliver conjugated dendrimeri cstructures to the nucleus of a cell. Other suitable peptides with nuclear or nucleolar localization properties are described in, for example, Antopolsky, et al., Bioconjugate Chem., 1999, 10, 598; Zanta, et al., Proc. Natl. Acad. Sci. USA, 1999 (simian virus 40 large tumor antigen); Hum. Mol. Genetics, 2000, 9, 1487; and FEBS Lett., 2002, 532, 36). In some embodiments, the delivery peptide for nucleus or nucleolar localization comprises at least three consecutive arginine residues or at least four consecutive arginine residues. Nuclear localization can also be facilitated by peptide conjugates containing RS, RE, or RD repeat motifs (Cazalla, et al., Mol. Cell. Biol., 2002, 22, 6871). In some embodiments, the peptide conjugate contains at least two RS, RE, or RD motifs.

Localization of oligonucleotides to the ER can be effected by, for example, conjugation to the signal peptide KDEL (SEQ ID NO: 18) (Arar, et al., Bioconjugate Chem., 1995, 6, 573; Pichon, et al., Mol. Pharmacol. 1997, 51, 431)

Cytoplasmic localization of oligomeric compounds can be facilitated by conjugation to peptides having, for example, a nuclear export signal (NES) (Meunier, et al., Nucleic Acids Res., 1999, 27, 2730). NES peptides include the leucine-rich NES peptides derived from HIV-1 Rev (Henderson, et al., Exp. Cell Res., 2000, 256, 213), transcription factor III A, MAPKK, PKI-alpha, cyclin B1, and actin (Wade, et al., EMBO J., 1998, 17, 1635) and related proteins. Antimicrobial peptides, such as dermaseptin derivatives, can also facilitate cytoplasmic localization (Hariton-Gazal, et al., Biochemistry, 2002, 41, 9208). Peptides containing RG andlor KS repeat motifs can also be suitable for directing oligomeric compounds to the cytoplasm. In some embodiments, the peptide conjugate moieties contain at least two RG motifs, at least two KS motifs, or at least one RG and one KS motif.

In a particular embodiment, like delivery peptides, nucleic acids can also serve or be looked at as conjugate moieties that can affect localization of conjugated oligomeric compounds in a cell. For example, nucleic acid conjugate moieties can contain poly A, a motif recognized by poly A binding protein (PABP), which can localize poly A- containing molecules in the cytoplasm (Gorlach, et al., Exp. Cell Res., 1994, 211, 400. In some embodiments, the nucleic acid conjugate moiety contains at least 3, at least 4, at least 5, at least 6, at least 7, at least 8, at least 9, at least 10, at least 15, at least 20, and at least consecutive A bases. The nucleic acid conjugate moiety can also contain one or more AU-rich sequence elements (AREs). AREs are recognized by ELAV family proteins which can facilitate localization to the cytoplasm (Bollig, et al., Biochem. Biophys. Res. Commun., 2003, 301, 665). Example AREs include WAUUUAW and sequences containing multiple repeats of this motif. In other embodiments, the nucleic acid conjugate moiety contains two or more AU or AUU motifs. Similarly, the nucleic acid conjugate moiety can also contain one or more CU-rich sequence elements (CREs) (Wein, et al., Eur. J. Biochem., 2003, 270, 350) which can bind to proteins HuD and/or HuR of the ELAV family of proteins. As with AREs, CREs can help localize conjugated oligomeric compounds to the cytoplasm. In some embodiments, the nucleic acid conjugate moiety contains the motif (CUUU)n, wherein, for example, n can be 1 to about 20, 1 to about 15, or 1 to about 11. The (CUW)n motif can optionally be followed or preceded by one or I more U. In some embodiments, n is about 9 to about 12 or about 11.

The nucleic acid conjugate moiety can also include substrates of hnRNP proteins (heterogeneous nuclear ribonucleoprotein), some of which are involved in shuttling nucleic acids between the nucleus and cytoplasm (e.g., nhRNP At and nhRNP K, see, e.g., Mill, et al., Mol. Cell Biol., 2001, AI, 7307). Some example hnRNP substrates include nucleic acids containing the sequence UAGGA/U or (GG)ACUAGC(A). Other nucleic acid conjugate moieties can include Y strings r or other tracts that can bind to, for example, hnRNP 1. In some embodiments, the nucleic acid conjugate moiety can contain at least 3, at least 4, at least 5, at least 6, at least 7, at least 8, at least 9, at least 10, at least 15, at least 20, and at least 25 consecutive pyrimidine bases. In other embodiments the nucleic acid conjugate moiety can contain greater than 50, greater than 60, greater than 70, greater than 80, greater than 90, or greater than 95 percent pyrimidine bases.

Other nucleic acid conjugate moieties can include pumilio (puf protein) recognition sequences such as described in Wang, et al., Cell, 2002, 110, 501. Example pumilio recognition sequences can include UGUANAUR, where N can be any base and R can be a purine base.

Localization to the cytoplasm can be facilitated by nucleic acid conjugate moieties conataining AREs and/or CREs. Nucleic acid conjugate moieties serving as substrates of hnRNPs can facilitate localization of oligonucleotides to the cytoplasm (e.g., hnRNP AI or K) or nucleus (e.g., hnRNP I).

Addtionally, nucleus localization can be facilitated by nucleic acid conjugate moieties containing polypyrimidine tracts.

Many drugs, receptor ligands, toxins, reporter molecules, and other small molecules can serve as conjugate moieties. Small molecule conjugate moieties often have specific interactions with certain receptors or other biomolecules, thereby allowing targeting of conjugated oligomeric compounds to specific cells or tissues.

Example small molecule conjugate moieties include mycophenolic acid (inhibitor of inosine-5′-monophosphate dihydrogenase; useful for treating psoriasis and other skin disorders), curcumin (has therapeutic applications to psoriasis, cancer, bacterial and viral diseases). In further embodiments, small molecule conjugate moieties can be ligands of serum proteins such as human serum albumin (HSA). Numerous ligands of LISA are known and include, for example, arylpropionic acids, ibuprofen, warfarin, phenylbutazone, suprofen, carprofen, fenfulen, ketoprofen, aspirin, indomethacin, (S)-( )-pranoprofen, dansylsarcosine, 2,3,5-trliodobenzoic acid, flutenamic acid, folinic acid, benzothiadiazide, chlorothiazide, diazepines, indomethicin, barbituates, cephalosporins, sulfa drugs, antibacterials, antibiotics (e.g., puromycin and pamamycin), and the like. Oligonucleotide-drug conjugates and their preparation are described in, for example, WO 00/76554, which is incorporated herein by reference in its entirety and which can, be used to guide the person skilled in the art to prepare drug-dendrimeric structure conjugates.

In yet further embodiments, small molecule conjugates can target or bind certain receptors or cells. T-cells are known to have exposed amino groups that can form Schiff base complexes with appropriate molecules. Thus, small molecules containing functional groups such as aldehydes that can interact or react with exposed amino groups can also be suitable conjugate moieties. Tucaresol and related compounds can be conjugated to oligomeric compounds in such a way as to leave the aldehyde free to interact with T-cell targets. Interaction of tucaresol with T-cells in believed to result in therapeutic potentiation of the immune system by Schiff-base formation (Rhodes, et al., Nature, 1995, 377, 6544).

Reporter groups that are suitable as conjugate moieties include any moiety that can be detected by, for example, spectroscopic means. Example reporter groups include dyes, fluorophores, phosphors, radiolabels, and the like. In some embodiments, the reporter group is biotin, flourescein, rhodamine, coumarin, or related compounds. Reporter groups can also be attached to other conjugate moieties.

Other conjugate moieties can include proteins, subunits, or fragments thereof. Proteins include, for example, enzymes, reporter enzymes, antibodies, receptors, and the like. In some embodiments, protein conjugate moieties can be antibodies or fragments thereof (Kuijpers, et al., Bioconjugate Chem., 1993, 4, 94).

Antibodies can be designed to bind to desired targets such as tumor and other disease-related antigens. In further embodiments, protein conjugate moieties can be serum proteins such as HAS or glycoproteins such as asialoglycoprotein (Rajur, et al., Bioconjugate Chem., 1997, 6, 935).

Other conjugate moieties can include, for example, oligosaccharides and carbohydrate clusters such as Tyr-Glu-Glu- (aminohexyl GaINAc)3 (YEE(ahGalNAc); a glycotripeptide that binds to Gal/GaINAc receptors on hepatocytes, see, e.g., Duff, et al., Methods Eanzymol., 2000, 313, 297); lysine-based galactose clusters (e.g., L3G4; Biessen, et al., Dev. Cardovasc. Med., 1999, 214); and and cholane-based galactose clusters (e.g., carbohydrate recognition motif for asialoglycoprotein receptor). Further suitable conjugates can include oligosaccharides that can bind to carbohydrate recognition domains (CRD) found on the asiologlycoprotein-receptor (ASGP-R). Example conjugate moieties containing oligosaccharides and/or carbohydrate complexes are provided in U.S. Pat. No. 6,525,031, which is incorporated herein by reference in its entirity.

Intercalators and minor groove binders (MGBs) can also be suitable as conjugate moieties. In some embodiments, the MOB can contain repeating DPI (1,2-dThydro-3H-pyrrolo(2,3-e)indole-7-carboxylate) subunits or derivatives thereof (Lukhtanov, et al., Bioconjugate Chem., 1996, 7, 564 and Afonina, et al., Proc. Natl. Acad. Sci. USA, 1996, 93, 3199). Suitable intercalators include, for example, polycyclic aromatics such as naphthalene, perylene, phenanthridine, benzophenanthridine, phenazine, anthraquinone, acridine, and derivatives thereof. Hybrid intercalator/ligands include the photonuclease/intercalator ligand 6-[[[9-[[6-1(4-nitrobenzamido)hexyl]amino]acridin-4-yl]carbonyl]amino]hexanoyl pentafluorophenyl ester. This compound is both an acridine moiety that is an intercalator and a p-nitro benzamido group that is a photonuclease.

In further embodiments, cleaving agents can serve as conjugate moieties. Cleaving agents can facilitate degradation of target, such as target nucleic acids, by hydrolytic or redox cleavage mechamisms. Cleaving groups that can be I suitable as conjugate moieties include, for example, metallocomplexes, peptides, amines, enzymes, and constructs containing constituents of the active sites of nucleases such as imidazole, guanidinium, carboxyl, amino groups, etc.). Example metallocomplexes include, for example, Cu-terpyridyl complexes, Fe-porphyrin complexes, Ru-complexes, and lanthanide complexes such as various Eu(lIl) complexes (Hall, et al., Chem. Biol., 1994, 1, 185; Huang, et al., J. Biol. Inorg Chem., 2000, 5, 85; and Baker, et al., Nucleic Acids Res., 1999, 27, 1547). Other metallocomplexes with cleaving properties include metalloporphyrins and derivatives thereof. Example peptides with target cleaving properties include zinc fingers (U.S. Pat. No. 6,365,379; Lima, et al., Proc. Natl. Acad. Sci. USA, 1999, 96, 110010). Example constructs containing nuclease active site constituents include bisimiazole and histamine.

Cross-linking agents can also serve as conjugate moieties. Cross linking agents facilitate the covalent linkage of the conjugated dendrimeric structures with other compounds. In some embodiments, cross-linking agents can covalently link double-stranded nucleic acids, effectively increasing duplex stability and modulating pharmacokinetic properties. In some embodiments, cross-linling agents can be photoactive or redox active. Example cross-linking agents include psoralens which can facilitate interstrand cross-linking of nucleic acids by photoactivation (Lin, et al., Faseb J., 1995, 9, 1371). Other cross-linking agents include, for example, mitomycin C and analogs thereof (Maruenda, et al., Bioconjugate Chem., 1996, 7, 541; Maruenda, et al., Anti-Cancer Drug Des., 1997, 12, 473; and Huh, et al., Bioconjugate Chem., 1996, 7, 659). Cross-linking mediated by mitomycin C can be effected by reductive activation, such as, for example, with biological reductants (e.g., NADPH-cytochrome c reductase/NADPH system). Further photo-crosslinking agents include aryl azides such as, for example, N-hydroxysuccinTimidyl-4 azidobenzoate (HSAB) and N-succinimidyl-6(-4′-azido-2′-nitrophenyl amino)hexanoate (SANPAH). Aryl azides conjugated to oligonucleotides effect crosslinking with nucleic acids and proteins upon irradiation. They can also crosslink with carrier proteins (such as KLH or BSA).

Other suitable conjugate moieties include, for example, polyboranes, carboranes, metallopolyboranes, metallocarborane, derivatives thereof and the like (see, e.g., U.S. Pat. No. 5,272,250, which is incorporated herein by reference in its entirety).

Conjugate moieties can be attached to the oligomeric compound or to the dendrimeric structure directly or through a linking moiety (linker or tether). Linkers are bifunctional moieties that serve to covalently connect a conjugate moiety to a dendrimeric structure or to an oligomeric compound. In some embodiments, the linker comprises a chain structure or an oligomer of repeating units such as ethylene glyol or amino acid units or even oligonucleotide sequences can serve as or be looked at as linking moieties. The linker can have at least two functionalities, one for attaching to the dendrimeric structure or the oligomeric compound and the other for attaching to the conjugate moiety. Example linker functionalities can be electrophilic for reacting with nucleophilic groups, or nucleophilic for reacting with electrophilic groups. In some embodiments, linker functionalities include amino, hydroxyl, carboxylic acid, thiol, phosphoramidate, phophate, phosphite, unsaturations (e.g., double or triple bonds), and the like. Some example linkers include 8-amino-3,6-dioxaoctanoic acid (ADO), succinimidyl 4-(N-maleimidomethyl) cyclohexane-1-carboxylate (SMCC), 6 aminohexanoic acid (AMEX or AMA), 6-aminohexyloxy, 4-aminobutyric acid, 4 aminocyclohexylcarboxylic acid, succinimidyl 4-(N-maleimidomethyl)cyclohexane 1-carboxy-(6-amido-caproate) (LCSMCC), succinimidyl m-maleimido-benzoylate (MBS), succinimidyl N-maleimido-caproylate (EMCS), succinimidyl 6-(,6 maleimido-propionamido) hexanoate (SMPH), succinimidyl N-(a-maleimido acetate) (AMAS), succinimidyl 4-(p-maleimidophenyl)butyrate (SMPB), p-alanine (-ALA), phenylglycine (PHG), 4-aminocyclohexanoic acid (ACHC), p-(cyclopropyl) alanine (-CYPR), amino dodecanoic acid (ADC), alylene dials, polyethylene glycols, amino acids, and the like.

A wide variety of futher linker groups are known in the art that can be useful in the attachment of conjugate moieties to oligomeric ompounds or dendrimeric structures. A review of many of the useful linker groups can be found in, for example, Antisense Research and Applications, S. T. Crooke and B. Lebleu, Eds., CRC Press, Boca Raton, Fla., 1993, p. 303-350. Nelson, et al., Nuc. Acids Res. 1989, 17, 7187 describe a linking reagent for attaching biotin to the 3′-terminus of an oligonucleotide. This reagent, N-Fmoc-O DMT-3-amino-1,2-propanediol is commercially available from Clontech Laboratories (Palo Alto, Calif.) under the name 3′-Amine. It is also commercially available under the name 3′-Amino-Modifier reagent from Glen Research Corporation (Sterling, Va.). This reagent was also utilized to link a peptide to an oligonucleotide as reported by Judy, et al., Tetrahedron Letters 1991, 32, 879. A similar commercial reagent for linking to the 5′-terminus of an oligonucleotide is 5′ Amino-Modifier C6. These reagents are available from Glen Research Corporation (Sterling, Va.). These compounds or similar ones were utilized by Krieg, et al., Antisense Research and Development 1991, 1, 161 to link fluorescein to the 5′ terminus of an oligonucleotide. Other compounds such as acridine have been attached to the 3′-terminal phosphate group of an oligonucleotide via a polymethylene linkage (Asseline, et al., Proc. NatI. Acad. Sci. USA 1984, SI, 3297). Any of the above groups can be used as a single linker or in combination with one or more further linkers.

Linkers and their use in preparation of conjugates of oligomeric compounds are provided throughout the art such as in WO 96/11205 and WO 98/52614 and U.S. Pat. Nos. 4,948,882; 5,525,465; 5,541,313; 5,545,730; 5,552,538; 5,580,731; 5,486,603; 5,608,046; 4,587,044; 4,667,025, 5,254,469; 5,245,022; 5,112,963; 5,391723; 510,475; 5,512,667; 5,574,142; 5,684,142; 5,770,716; 6,096,875; 6,335,432; and 6,335,437, each of which is incorporated by reference in its entirety and which can serve as basis for the synthesis of dendrimeric bioconjugates.

For dendrimeric bioconjugates, conjugate moieties or linkers can be attached to any position of the dendrimeric structure which is available for linking, without interfering with the interaction capacities of the dendrimer with the oligomeric compound to be delivered. For example, when the dendrimeric structure is PAMAM, the free amino groups of the PAMAM dendrimer can be used for reaction with a linking group such as used in the examples described herein. By using different equivalents of linker or conjugate moieties to be coupled, the amount of conjugate moieties can be adapted for different dendrimeric structures. For PAMAM for example, multiple conjugate moieties are coupled to the dendrimer, while for the oligonucleotide dendrimers from the examples herein, one conjugate moiety is attached to the dendrimer.

For conjugates of conjugate moieties and oligonucleotides not being dendrimeric structures, diffeernt possibilities exist, as long as also the hybridization characteristics of the oligonucleotide are not disrupted. In some embodiments, conjugate moieties can be attached to the terminus of an oligomeric compound such as a 5′ or 3′ terminal residue of a nucleic acid. Conjugate moieties can also be attached to internal residues of the oligomeric compounds. For double-stranded oligomeric compounds, conjugate moieties can be attached to one or both strands. In some embodiments, a double-stranded oligomeric compound contains a conjugate moiety attached to the sense strand of the hybridization complex of the method of the presently disclosed subject matter. In other embodiments, a double-stranded oligomeric compound contains a conjugate moiety attached to the antisense strand.

In some examples known in the prio art, conjugate moieties can be attached to heterocyclic base moieties (e.g., purines and pyrimidines), nucleotide sugar subunits, internucleotide linkages (e.g. phosphodiester linkages) of nucleic acid molecules. Conjugation to purines or derivatives thereof can occur at any position including, endocyclic and exocyclic atoms. In the prior art, the 2-, 6-, 7-, or 8-positions of a purine base can be attached to a conjugate moiety. Conjugation to pyrimidines or derivatives thereof can also occur at any position. In some instances, the 2-, 5-, and 6-positions of a pyrimidine base can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O. N. or S atom bound to the phosphorus atom. For amine-or amide-containing internucleosidic linkages (e.g., PNA), the conjugate moiety can be attached to the nitrogen atom of the amine or amide or to an adjacent carbon atom.

Methods for preparing conjugates of dendrimeric structures. Preparation methods of conjugates of dendrimeric structures are described herein and are also known in the art. As for the oligonucleotide conjugates, generally, a dendrimeric structure is attached to a conjugate moiety by contacting a reactive group (e.g., OH, SH, amine, carboxyl, aldehyde, and the like) on the dendrimeric structure with a reactive group on the conjugate moiety. In some embodiments, one reactive group is electrophilic and the other is nucleophilic. Other embodiments encompass the reaction between a carboxylgroup or activated carboxylgroup and amines or substituted amines in order to create amides.

For the preparation of the cholesterol-oligonucleotide dendrimeric bioconjugate of the presently disclosed subject matter, the following steps are involved: (i) derivatization of cholesterol in order to obtain a free carboxylgroup, (ii) coupling to the derivatized cholesterol Fmoc-lysine-OMe or an analoguous structure comprising a protected amino group and a protected carboxylgroup which are separately cleavable, (iii) deprotection of the carboxylgroup, (iv) coupling the obtained compound to a solid phase through an acid labile coupling, like an ester, (v) coupling a linker with a free hydroxyl as reactive group and (vi) perform standard oligonucleotide synthesis followed by cleaving the compound of from the solid phase. For obtaining a dendrimer, step (iv) is first followed by (vi a) which comprises the coupling of a compound with two protected amino functions and a free carboxylgroup like (Fmoc)₂-Lys-OH.

For the derivatization of cholesterol to contain a free carboxylgroup, cholesterolacetic acid was prepared. The preparation hereof comprises te steps of (i) reacting cholesterol with bromoacetaldehyde diethylacetal in order to create Cholesterylacetaldehyde diethylacetal, (ii) deprotection to cholesterolacetaldehyde and (iii) oxidation of the aldehyde to a carboxyacid, namely cholesterylacetic acid.

For the preparation of Tat-PAMAM conjugates, the free amino groups of the PAMAM dendrimer were first reacted with sulfo-LC-SMPT, a crosslinking reagent which reacts with amino groups and allows sulfur containing compounds to bind to it and secondly, the Cysteine derivatized (linker) Tat peptide was then reacted.

Chemistry Defined. The term “alkyl” as used herein refers to a normal, secondary, or tertiary hydrocarbon. “Short chain” or “lower” alkyl refers to C₁-C₆ alkyl, while alkyl could be longer such as C₁-C₁₂. Examples are methyl, ethyl, 1-propyl, 2-propyl, 1-butyl, 2-methyl-1-propyl(i-Bu), 2-butyl (s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl.

As used herein and unless otherwise stated, the term “cycloalkyl” means a monocyclic saturated hydrocarbon monovalent radical having in a particular embodiment from 3 to 10 carbon atoms, such as for instance cyclopropyl, cyclobutyl, cyclopentyl, cyclohexyl, cycloheptyl, cyclooctyl and the like, or a C₇₋₁₀ polycyclic saturated hydrocarbon monovalent radical having from 7 to 10 carbon atoms such as, for instance, norbornyl, fenchyl, trimethyltricycloheptyl or adamantyl.

The terms “alkenyl” and “cycloalkenyl” as used herein is normal, secondary or tertiary and respectively cyclic hydrocarbon with at least one site (usually 1 to 3, preferably 1) of unsaturation, i.e. a carbon-carbon, sp2 double bond, where in a particular embodiment, alkenyl has from C₂-C₁₂ and cycloalkenyl from C₃-C₁₀. Examples include, but are not limited to: ethylene or vinyl (—CH═CH2), allyl (—CH2CH═CH2), cyclopentenyl (—C5H7), and 5-hexenyl (—CH2CH2CH2CH2CH═CH2). The double bond may be in the cis or trans configuration.

The terms “alkynyl” and “cycloalkynyl” as used herein refer respectively to normal, secondary, tertiary or the cyclic hydrocarbon with at least one site (usually 1 to 3, preferably 1) of unsaturation, i.e. a carbon-carbon, sp triple bond, where in a particular embodiment, alkynyl has from C₂-C₁₂ and cycloalkenyl from C₃-C₁₀. Examples include, but are not limited to: acetylenic and propargyl.

The term “aryl” as used herein means a aromatic hydrocarbon radical of 6-20 carbon atoms derived by the removal of hydrogen from a carbon atom of a parent aromatic ring system. Typical aryl groups include, but are not limited to 1 ring, or 2 or 3 rings fused together, radicals derived from benzene, naphthalene, spiro, anthracene, biphenyl, and the like.

“Arylalkyl” or “aralkyl” as used herein refers to an alkyl radical in which one of the hydrogen atoms bonded to a carbon atom, typically a terminal or sp3 carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. “Alkylaryl” or “Alkaryl” refers to an aryl radical in which one of the hydrogen atoms bonded to a carbon atom is replaced with an alkyl radical.

“Heterocyclic” refers to ring structures comprising “heteroatoms” such as N, S or O. The term “heterocyclic” thus for example refers to pyridyl, dihydroypyridyl, tetrahydropyridyl (piperidyl), thiazolyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, and many others.

Many combinations of the above terms/structures are possible and thus create for example heterocycloalkyl wherein a heterocyclic rind is substituted with an alkyl and others like heterocycloalkaryl, etc.

As used herein and unless otherwise stated, the term halogen means any atom selected from the group consisting of fluorine, chlorine, bromine and iodine.

Any substituent designation that is found in more than one site in a compound of this presently disclosed subject matter shall be independently selected.

Naturally occurring “amino acid” residues are those residues found naturally in plants, animals, or microbes, especially proteins thereof. Polypeptides most typically will be substantially composed of such naturally occurring amino acid residues. These amino acids are glycine, alanine, valine, leucine, isoleucine, serine, threonine, cysteine, methionine, glutamic acid, aspartic acid, lysine, hydroxylysine, arginine, histidine, phenylalanine, tyrosine, tryptophan, proline, asparagine, glutamine and hydroxyproline. Additionally, unnatural amino acids, for example, valanine, phenylglycine and homoarginine are also included.

Screening, Target Validation, Drug Discovery, Kits, Research Reagents, Diagnostics, and Therapeutics. The methods of the presently disclosed subject matter can be used in research such as for screening, target validation, drug discover, etc. in order to increase the uptake of the oligomeric compound which can modulate the expression of a selected protein. For use in drug discovery and target validation, an increase in the delivery of oligomeric compounds to the cells through the method of the presently disclosed subject matter can be used to elucidate relationships that exist between proteins and a disease state, phenotype, or condition. The methods and compounds of the presently disclosed subject matter can additionally be utilized for diagnostics, therapeutics, prophylaxis and as research reagents and kits. Such uses allows for those of ordinary skill to elucidate the function of particular genes or to distinguish between functions of various members of a biological pathway.

For use in kits and diagnostics, the methods, compounds or compositions or reagents of the presently disclosed subject matter, either alone or in combination with other compounds or therapeutics, can be used as tools in differential and/or combinatorial analyses to elucidate expression patterns of a portion or the entire complement of genes expressed within cells and tissues. As one non-limiting example, expression patterns within cells or tissues treated with one or more methods, compounds or compositions of the presently disclosed subject matter are compared to control cells or tissues not treated with the compounds or compositions and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns.

The methods, compounds and compositions or reagents of the presently disclosed subject matter can be applied for preventive or therapeutic reasons. Antisense oligomeric compounds have been employed as therapeutic moieties in the treatment of disease states in animals, including humans. Antisense oligonucleotide drugs, including ribozymes, have been safely and effectively administered to humans and numerous clinical trials are presently underway. It is thus established that oligomeric compounds can be useful therapeutic modalities that can be configured to be usefull in treatment regimes for the treatment of cells, tissues and animals, especially humans and that increasing the delivery of the oligomeric compounds to cells is favorable for the therapy.

For therapeutics, an animal, such as a human, suspected of having a disease or disorder that can be treated by modulating the expression of a selected protein is treated by administering the compounds and compositions of the presently disclosed subject matter or by applying the methods of the presently disclosed subject matter. For example, in one non-limiting embodiment, the methods comprise the step of administering to the animal in need of treatment, a therapeutically effective amount of a composition of the presently disclosed subject matter for the inhibition of the expression of a protein. The compositions of the presently disclosed subject matter effectively inhibit the activity of the protein or inhibit the expression of the protein. In some embodiments, the activity or expression of a protein in an animal is inhibited by about 10%. In further embodiments, the activity or expression of a protein in an animal is inhibited by about 30%. In yet further embodiments, the activity or expression of a protein in an animal is inhibited by 50% or more.

For example, the reduction of the expression of a protein can be measured in serum, adipose tissue, liver or any other body fluid, tissue or organ of the animal. In some embodiments, the cells contained within the fluids, tissues or organs being analyzed contain a nucleic acid molecule encoding a protein and/or the protein itself.

The compounds and compositions of the presently disclosed subject matter can be utilized in pharmaceutical compositions by adding an effective amount of the compound or composition to a suitable pharmaceutically acceptable diluent or carrier. Use of the oligomeric compounds and methods of the presently disclosed subject matter may also be useful prophylactically.

Formulations, compositions, pharmaceutical compositions, combinations and dosing. The methods of the presently disclosed subject matter comprise for example compositions of hybridization complexes with liposomes or dendrimeric bioconjugates with oligomeric compounds.

The compounds, reagents or compositions of the presently disclosed subject matter may furter be mixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption thereof.

The compounds and compositions of the presently disclosed subject matter encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the presently disclosed subject matter, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions.

The term “pharmaceutically acceptable salts” refers to physiologically and pharmaceutically acceptable salts of the compounds, reagents and compositions of the presently disclosed subject matter: i.e., salts that retain the desired biological activity of the parent compound and do not impart undesired toxicological effects thereto. For oligonucleotides, pharmaceutically acceptable salts and their uses are further described in U.S. Pat. No. 6,287,860, which is incorporated herein in its entirety.

The presently disclosed subject matter also includes pharmaceutical compositions and formulations that include the compounds and compositions of the presently disclosed subject matter. The pharmaceutical compositions of the presently disclosed subject matter may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or i intravenkicular, administration. Pharmaceutical compositions and formulations for topical administration may include transdermal patches, ointments, lotions, creams, gels, drops, suppositories, sprays, liquids and powders. Conventional pharmaceutically acceptable carriers, aqueous, powder or oily bases, thickeners and the like may be necessary or desirable. Coated condoms, gloves and the like may also be useful.

The pharmaceutical formulations of the presently disclosed subject matter, which may conveniently be presented in unit dosage form, may be prepared according to conventional techniques well known in the pharmaceutical industry. Such techniques include the step of bringing into association the active ingredients with the pharmaceutical carrier(s) or excipient(s).

In general, the formulations are prepared by uniformly and intimately bringing into association the active ingredients with liquid carriers or finely divided solid carriers or both, and then, if necessary, shaping the product.

The compounds and compositions of the presently disclosed subject matter may be formulated into any of many possible dosage forms such as, but not limited to, tablets, capsules, gel capsules, liquid syrups, soft gels, suppositories, and enemas The compositions of the presently disclosed subject matter may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions or formulations of the presently disclosed subject matter include, but are not limited to, solutions, emulsions, foams and liposome-containing formulations. The pharmaceutical compositions and formulations of the presently disclosed subject matter may comprise one or more penetration enhancers, carriers, excipients or other active or inactive ingredients.

Emulsions are typically heterogenous systems of one liquid dispersed in another in the form of droplets usually exceeding 0.1 1lm in diameter. Emulsions may contain additional components in addition to the dispersed phases, and the active drug that may be present as a solution in either the aqueous phase, oily phase or itself as a separate phase. Microemulsions are included as an embodiment of the presently disclosed subject matter. Emulsions and their uses are well known in the art and are further described in U.S. Pat. No. 6,287,860, which is incorporated herein in its entirety.

Formulations of the presently disclosed subject matter include in a preferred embodiment liposomal formulations As used in the presently disclosed subject matter, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged oligonucleotides to form a stable complex. Liposomes that are pH-sensitive or negatively charged are believed to entrap oligonucleotides rather than complex with it. Both cationic and noncationic liposomes have been used to deliver oligonucleotides to cells. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are furler described in U.S. Pat. No. 6,287,860, which is incorporated herein in its entirety. Examples of liposomes are lipofectamine (i.e. 2000).

The pharmaceutical formulations and compositions of the presently disclosed subject matter may also include surfactants. The use of surfactants in drug products, formulations and in emulsions is well known in the art. Surfactants and their uses are further described in U.S. Pat. No. 6,287,860, which is incorporated herein in its entirety. In one embodiment, the presently disclosed subject matter employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration. Formulations for topical administration include those in which the compounds of the presently disclosed subject matter are in admixture with a topical delivery agent such as lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, compounds and compositions of the presently disclosed subject matter may be encapsulated within liposomes or may form complexes thereto, in particular to cationic liposomes. Alternatively, they may be complexed to lipids, in particular to cationic lipids. Fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets Thickeners, flavoring agents, diluents, emulsifiers, dispersing aids or binders may be desirable. Oral formulations are those in which the compounds of the presently disclosed subject matter are administered in conjunction with one or more penetration enhancers surfactants and chelators. Surfactants include fatty acids and/or esters or salts thereof, bile acids and/or salts thereof. Bile acids/salts and fatty acids and their uses are further described in U.S. Pat. No. 6,287,860, which is incorporated herein in its entirety. Also included are combinations of penetration enhancers, for example, fatty acids/salts in combination with bile acids/salts. A example combination is the sodium salt of lauric acid, capric acid and UDCA. Further penetration enhancers include polyoxyethylene-9-lauryl ether, polyoxyethylene-20-cetyl ether. Compounds and compositions of the presently disclosed subject matter may be delivered orally, in granular form including sprayed dried particles, or complexed to form micro or nanoparticles. Complexing agents and their uses are further described in U.S. Pat. No. 6,287,860, which is incorporated herein in its entirety. Certain oral formulations for oligonucleotides and their preparation are described in detail in U.S. application Ser. No. 09/108, 673 (filed Jul. 1, 1998), Ser. No. 09/315,298 (filed May 20, 1999) and Ser. No. 10/071,822, filed Feb. 8, 2002, each of which is incorporated herein by reference in their entirety.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excpents.

Certain embodiments of the presently disclosed subject matter provide pharmaceutical compositions containing one or more of the compounds and compositions of the presently disclosed subject matter and one or more other agents that have a therapeutic activity for a certain disorder. Therefore, combinations of compounds and compositions of the presently disclosed subject matter and other drugs are also within the scope of this presently disclosed subject matter.

In another related embodiment, compositions of the presently disclosed subject matter may contain one or more of the compounds and compositions of the presently disclosed subject matter targeted to a first nucleic acid and one or more additional compounds targeted to a second nucleic acid target or to a different region of the same nucleic acid target. Two or more combined compounds may be used together or sequentially.

The formulation of therapeutic compounds and compositions of the presently disclosed subject matter and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds or composition of the presently disclosed subject matter, and can generally be estimated based on EC₅₀ found to be effective in in vitro and in vivo animal models.

While the presently disclosed subject matter has been described with specificity in accordance with certain of its embodiments, the following examples serve only to illustrate the presently disclosed subject matter and are not intended to limit the same.

### EXAMPLES

The following Examples provide illustrative embodiments. Certain aspects of the following Examples are described in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### General Procedures

Oligonucleotides and analogs used for the performing of the methods of the presently disclosed subject matter were prepared by methods known in the art for the synthesis of nucleic acid molecules or were purchased from companies offering such oligonucleotides.

Cells: NIH 3T3 cells stably transfected with a plasmid containing the human MDR1 gene (pSK1 MDR or NIH 3T3 MDR) were gift from M.M. Gottesman, National Cancer Institute, Bethesda MD [Kane S E et al. Gene 1989; 84: 439-446]. The MDR-3T3 cells were grown in DMEM (Dulbecco minimal cultured media) medium containing 10% FBS (fetal bovine serum) and 60 mg/ml colchicine if needed. Cells were maintained in a humidified atmosphere of 95% air, 5% CO₂ at 37° C.

Treatment of cells with antisense oligonucleotides, hybridization complexes, oligomeric compounds or dendrimeric bioconjugate compositions: The experimental protocols were as those previously described in Alahari S K et al. J Pharmacol Exp Ther 1998, 286, 419-428. Briefly, MDR 3T3 cells were grown in 162 mm flasks to 95% confluency and then seeded onto six well plates at 3×10⁵/well in 10% FBS/DMEM and incubated 24 hr. The cells were washed twice with PBS. Complexes between sense strand and antisense strand or between dendrimers and oligomeric compounds such as the antisense strand were prepared (in 10 mM TRIS, 50 mM NaCl, 1 mM EDTA) by heating the solutions at 80° C. and cooling down to room temperature for 15 minutes. The complexation of the double stranded dendrimeric form of the antisense or of the double stranded and single stranded antisense oligonucleotides was done with Lipofectamine 2000 (Invitrogene) in OptiMEM according to standard procedure. The complexes (with cationic lipids) were added to cells in 10% FBS/DMEM. The cells were incubated with complexes at 37° C. for 4 h or overnight. Then the cells were rinsed and further incubated for 68 h in 2% FBS/DMEM. All conditions were performed in triplicate. The P-glycoprotein percent shift is calculated from the lipofectamine 2000 control shift. Oligonucleotide concentrations ranged from 0.025 to 0.1 μM in various experiments (see Legends).

Pharmacological effects of treatment with antisense oligonucleotides, hybridization complexes, oligomeric compounds or dendrimeric bioconjugate compositions. Analysis of the pharmacological effects of oligomeric compounds or dendrimeric delivery was based on a previously described assay (Alahari, S. K. et al. (1996) Mol. Pharmacol. 50, 508-519.) involving antisense inhibition of P-glycoprotein expression in mouse 3T3 cells stably transfected with the human MDR1 gene. The cell surface expression of P-glycoprotein was determined by immunostaining and quantitation by flow cytometry, as described (Alahari, S. K. et al. (1998) J. Pharmacol. Exp. Ther. 286, 419-428.). Briefly, the MRK16 anti-P-glycoprotein antibody (Kamiya Biochemicals), which is directed against an external epitope, was employed as the primary antibody. After the incubation, the cells were washed twice, in 10% FBS/PBS. The level of Phycoerythrin (PE) conjugated goat anti-mouse IgG (Sigma, St. Louis, Mo. was used as the second antibody) in viable cells (viability determined by light scatter) was quantitated using the Summit V3.0 software application (Cytomation Inc.) on a Becton Dickinson flow cytometer.

### Example 2

### Double-Stranded Delivery of Oligonucleotides

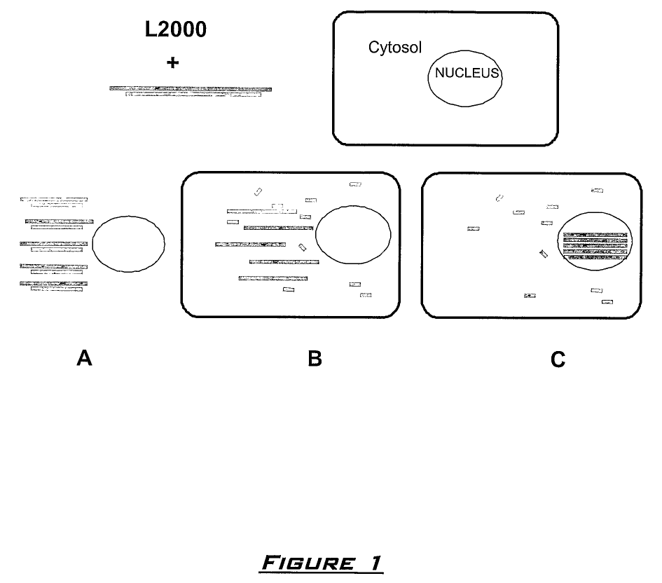

As an example of the double-stranded delivery of antisense oligonucleotides, we investigated the possibility of delivering a chemically modified antisense oligonucleotide together with its sense sequence in the DNA-form, using cationic lipids as an additional delivery agent. Neither part of the delivery system (sense DNA and cationic lipid) is covalently bond to the antisense oligonucleotide. The sense-DNAs have different lengths, as the stability of the duplex with the antisense oligonucleotide may influence the biological activity. Thus, the delivery of chemically modified antisense sequences complexed with a shorter unmodified sense sequence as a duplex was investigated. The antisense sequence is chemically modified to increase stability against enzymatic degradation. A gapmer was used in order to maintain RNaseH activity involved in the antisense mechanism. The sense sequence is an unmodified oligodeoxynucleotide which is prone to degradation by phosphodiesterases. It was reasoned that, once taken up by cells, the sense oligodeoxynucleotide should be easily degraded and liberate the antisense oligonucleotide for hybridization with its RNA target (FIG. 1). Using this approach, and targeting the MDRI gene, we demonstrated significantly stronger inhibition of P-glycoprotein expression compared to the effect with traditional delivery of a single strand oligonucleotide. P-glycoprotein is an ATP-driven trans-membrane pump that can expel a wide variety of drug molecules from cells. Over-expression of P-glycoprotein in tumor cells confers a multi-drug resistance (MDR) phenotype that can impede the effectiveness of cancer chemotherapy. Thus, a number of approaches have been devised to attempt to reverse the MDR phenotype, either through inhibition of P-glycoprotein function [i.e. Dalton W S et al. Cancer 1995; 75: 815-820.], or through reduction in its expression using molecular techniques [Scanlon K J et al. Proc Natl Acad Sci USA 1994; 91: 11123-11127; Kobayashi H et al. Cancer Res 1994; 54: 1271-1275.]. We and others have previously sought to inhibit expression of P-glycoprotein message and protein using antisense oligonucleotides [Alahari S K et al. Mol Pharmacol 1996; 50: 808-8196; Alahari S K et al. J Pharmacol Exp Ther 1998; 286: 419-428; Corrias M V et al. Nucleic Acids Res 1992; 12: 1431-1438; Efferth T et al. Oncology 1993; 50: 303-308; Cucco C et al. Cancer Res 1996; 56: 4332-4337] but with only moderate success. The new strategy that was invented, significantly improves the delivery and pharmacological effects of antisense oligonucleotides (in this example targeted to MDR1) as compared to the traditional single stranded approach.

The procedures used were as folowing:

Oligonucleotides: 2′-O-methyl gapmer RNA (gap phosphorothioate DNA residues indicated in bold) 20-mer anti-MDR1 5′-d(CCA-UCC-CGA-CCT-CGC-GCU-CC)-3′ and mismatch 5′-d(CCA-UAC-CAA-CAT-CAC-GCU-CC)-3′ oligonucleotides with and without FITC marker on the 5′ end were synthesized by RNA-Tec. Regular, unmodified oligodeoxynucleotides complementary to the antisense sequence: including 14-mer-5′ -d (G-CGC-GAG-GTC-GGG-A)-3′, 16-mer-5′-d(AG-CGC-GAG-GTC-GGG-AT)-3′, and 18-mer-5′-d(GAG-CGC-GAG-GTC-GGG-ATG)-3′, with and without TAMRA fluorescence marker were from Sigma Genosys and from the Oligonucleotide Facility of the UNC Linberger Cancer Center.

Uptake and sub-cellular distribution of peptide-oligonucleotide conjugates: Cells treated with fluorescent oligonucleotides or peptide-oligonucleotide conjugates were harvested by trypsinization and then examined by fluorescence microscopy or by flow cytometry. For microscopy, cells resuspended in 1 ml 10% FCS/DMEM were incubated for 6 hr on fibronectin-coated cover slips. The fluorescence patterns were analyzed on an Olympus Confocal FV300 fluorescent microscope with 60×-oil immersion objective. Images were captured with a slow scan charge-coupled device 3CCD Video Camera System interfaced to a PC using the Olympus Fluoview Software. For flow cytometry analysis cells were resuspended in 500 μl PBS and measured for the accumulation of TAMRA marker using a Becton Dickinson flow cytometer with Cicero software (Cytomation, Inc.).

Toxicity of antisense treatment: The cytotoxicity of the various treatments used in the oligonucleotide experiments was evaluated by cell enumeration. Briefly, the cells treated with antisense oligonucleotides in single or double stranded format were harvested by trypsinization at different time points after treatment and then analyzed by using an electronic particle counter (Elzone 80, Micro-meritics). Cell counts were normalized to untreated control.

Other procedures used for evaluation of the delivery of oligomeric compounds and their activity (i.e. cells, treatment with oligomeric compound and evaluation) were performed as described in the general procedures section hereof.

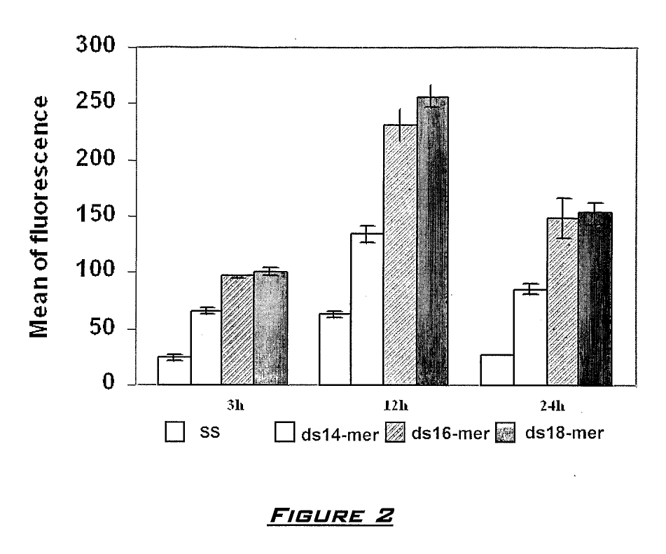

The 2′-O-methyl gapmer RNA and complementary unmodified oligodeoxynucleotides strand were hybridized together, complexed with Lipofectamine 2000 and introduced to the cells. As a control single stranded antisense 2′-O-methyl gapmer RNA complexed with Lipofectamine 2000 was used. We used fluorescently labeled antisense, but not sense strand to evaluate uptake efficiency and compare single and double stranded delivery. We also evaluated the optimal length of sense strand, ranging from a 14-mer to a 18-mer. As presented in FIG. 2, the uptake of the double stranded oligonucleotide is significantly higher than for the single stranded. The sense strand 16-and 18-mers seem to be the best with regard to promoting uptake. The difference between single stranded and double stranded delivery was four fold after 3 hours incubation and reached 6 times after 24 hours incubation. There was no significant difference in uptake performance between 16-mer and 18-mer duplexes.

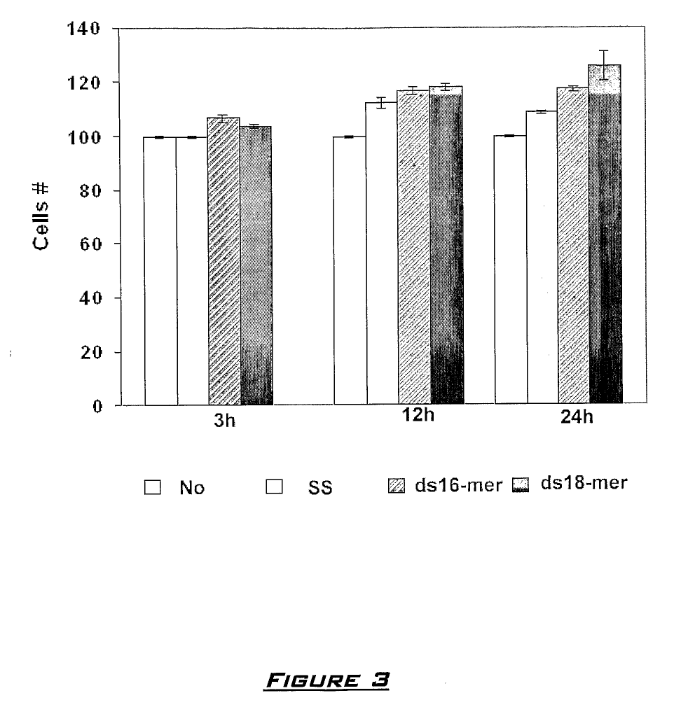

In parallel, using the same time points used for the uptake analysis, we evaluated the toxicity of double stranded delivery. We counted the number of live cells after 3, 12 and 24 hours and normalized the data to untreated control. As shown in FIG. 3, the proposed strategy doesn't have a negative impact on the number of living cells, rather some increase in the number of living cells for both 16-mer and 18-mer duplexes was observed.

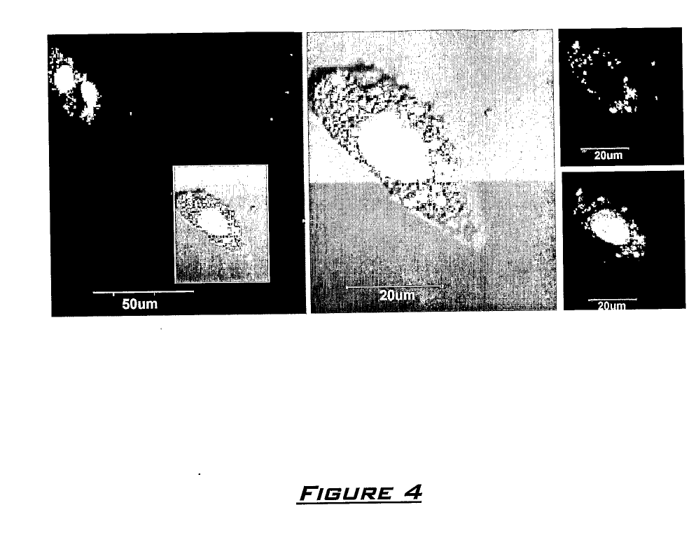

To show actual cellular localization of antisense and sense strand after double stranded delivery we analyzed cells after 24 hours of incubation of the 18-mer sense/ 20-mer antisense duplex (also with Lipofectamine 2000). We labeled the antisense strand with fluorescein and the sense strand with Texas Red and analyzed the cell images using a confocal microscope. As presented in FIG. 4 (and as expected) there is a strong green fluorescence from the antisense strand in the nucleus. The sense strand was not transported to the nucleus as evidenced by a lack of fluorescence in the nucleus. The red signal from the sense strand stays in the cytoplasm and it is most probably still in duplex form with antisense strand as green and red spots are co-localized (however this assumption needs to be evaluate in the future studies).

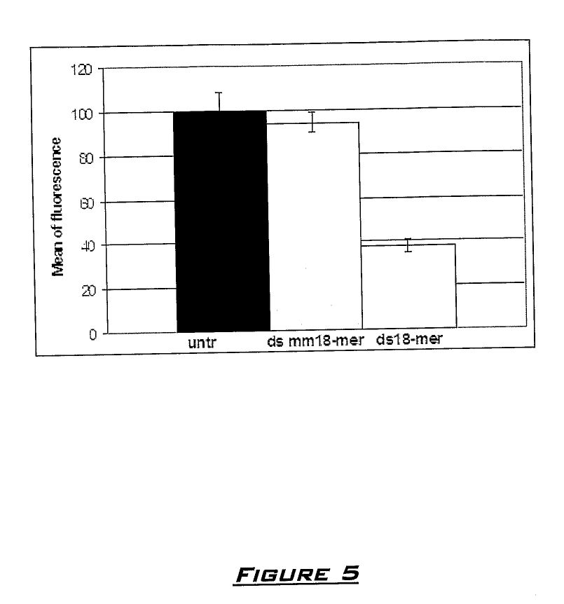

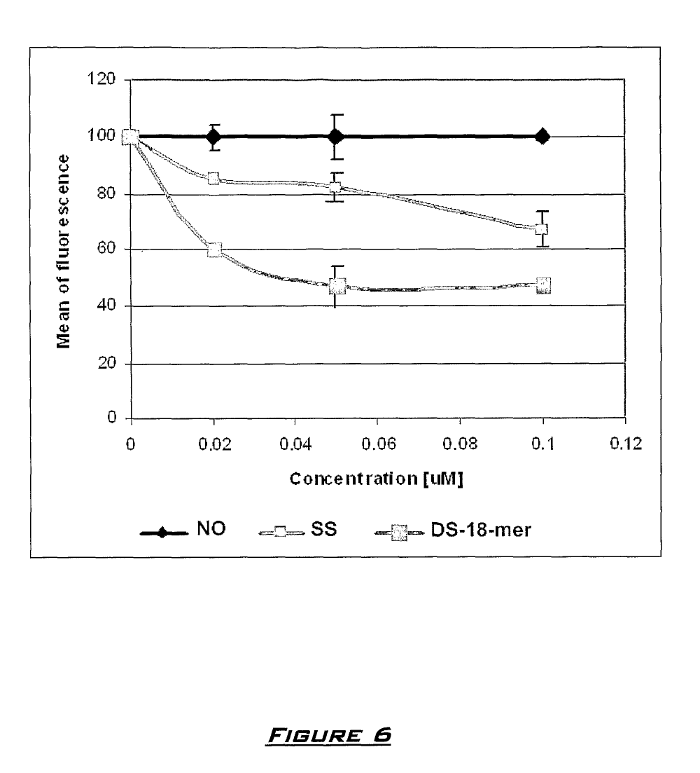

As final tests of double stranded delivery approach, we evaluated also the double stranded mismatch duplex and the inhibition of the P-glycoprotein expression by single and double stranded oligonucleotides. As shown in FIG. 5, the mismatch reference duplex has no activity at all on P-glycoprotein expression (compared to the correct double stranded antisense duplex). The normalized dose response inhibition of the P-glycoprotein expression is presented in FIG. 6. As shown, there is a significantly greater reduction of P-glycoprotein expression in the samples treated with the double-stranded complexes as opposed to the single stranded samples, particularly at low concentrations (0.025 μM and 0.05 μM). These results correlate well with uptake experiments and indicated that double stranded delivery leads to better uptake of the antisense oligonucleotides and as a result stronger biological response to antisense treatment.

From these experiments, we conclude that delivery of a stable antisense oligonucleotide together with its complementary DNA sequence in unstable form (and in the presence of Lipofectamine 2000) increases the uptake and the biological activity of the antisense oligonucleotide. This approach may provide a simple and effective means for enhancing the delivery of pharmacologically active oligonucleotides in cells. At this point the mechanism underlying this effect is unknown. It is not clear if increased initial uptake, reduced release from the cell, or increased stability is the key factor for explaining the effect of this formulation; these issues will be pursued in subsequent studies together with its applicability in vivo. Also the role of the cationic lipid and the possibility of chemically modifying the sense strand with a reporter group will be envisaged. In view of current results, a question may be asked as to whether the somewhat higher biological activity of siRNA (as compared to antisense) may be partly due to a better delivery process, since siRNA is also delivered as a duplex. An interesting question coming out of this research deals with the explanation of the biological activity of siRNA when the antisense strand is chemically modified. Such molecules may also be considered as antisense oligonucleotides, delivered with their biological degradable sense-RNA, and questions should be asked about the mode of action of such duplexes.

### Example 3

### Double-Stranded Antisense Oligonucleotide Delivery through Use of a Cholesterol-Oligonucleotide Dendrimeric Bioconiugate

As an example, cholesterol modified mono-, di-and tetrameric oligonucleotides (conjugate moiety-dendrimeric oligonucleoitde structure) were synthesized and hybridized with antisense oligonucleotides to study their incorporation in cationic liposomes together with the influence of this dendrimeric delivery system on biological activity. This oligonucleotide formulation gives a significant increase in the inhibition of P-glycoprotein expression in a cellular system.

Using a linker based on lysine chemistry, we were able to synthesize monomeric, dimeric and tetrameric oligonucleotide constructs covalently linked to a cholesterol moiety. These monomeric and dendrimeric sense DNA were complexed/hybridized with enzymatic stable (modified) antisense oligonucleotides targeting P-glycoprotein expression.

The synthesis was tested using an 11-mer as well as an 18-mer sense-DNA. For biological activity determination, we selected the 18-mer as this length seems to be optimal for this purpose (Astriab-Fisher, A et al. (2004) Biochem. Pharmacol. 68, 403-407). The antisense oligonucleotide that is used as model is a phosphorothioate 20 gapmer consisting of 2′-MethylOxyEthyl (2′-MOE) nucleotides in the flanks and phosphorothioate DNA (in bold) in the middle (5′-CCAUCCcgacctcgCGCUCC-3′). The phosphorothioate DNA gap of 8 nucleotides allows RNaseH to play a role in the mode of action of the antisense oligonucleotide. The phosphorothioate 2′-MOE flanks render the oligonucleotide stable against enzymatic degradation. This sequence has been demonstrated to inhibit P-glycoprotein expression in a cellular system (Astriab-Fisher, A. et al. (2000) Biochem. Pharmacol. 60, 83-90).

3.1 Procedures for the Synthesis of Cholesterol Dendrimers

General Chemistry. ¹H and ¹³C NMR spectra were recorded at 200 MHz with a Varian Gemini 200 in CDCl₃. ¹³C-assignments were based on the usual shift increments and calculus values and where necessary confirmed by APT-experiments. Assignments of quaternary carbons of the aromatic rings were based on comparison with other compounds and were mostly tentative. Electron impact mass spectra in positive (EI+) and negative ion mode (EI−) were acquired on a Kratos MS50TC mass spectrometer (Kratos, Manchester, UK), using MASPEC II data acquisition software (Mass Spectrometry Services Ltd., Manchester, UK). Electrospray ionization mass spectra in negative ion mode (ESI−) were recorded on a quadruple orthogonal acceleration/time-of-flight mass spectrometer (Q-TOf-2, Micromass, Manchester, UK). Absorption spectra were recorded with a Perkin-Elmer Lambda 40 spectrophotometer (Wellesley, Mass.) and fluorescence spectra with a Spex Fluorolog 3-22 fluorimeter (Jobin Yvon-Spex Instruments S. A., Inc., Edison, N.J.). Precoated Machery-Nagel Alugram® SiIG/UV 254 plates were used for TLC and spots were examined with UV light and a sulfuric acid/anisaldehyde spray. Column chromatography was performed on Acros silica gel (60-200 nm). Anhydrous solvents were obtained as follows: THF was refluxed on LiAlH₄ overnight and was distilled; dichloromethane (DCM) was stored on calcium hydride, refluxed and distilled; pyridine, triethylamine (TEA) and N,N-diisopropylethylamine (DIEA) were refluxed overnight on potassium hydroxide and distilled. N,N-dimethylformamide (DMF) was stored on activated molecular sieves (4 Å) for 3 days and was tested for absence of dimethylamine by the bromophenol test prior to use. DCM, DMF, acetic anhydride (Ac₂O) and pyridine were obtained from BDH (Poole, England). Piperidine, trifluoroacetic acid (TFA), diisopropylcarbodiimide (DIC), 1-hydroxybenzotriazole (HOBt), 1-methylimidazole (NMI), di-te/1-butyl dicarbonate [(Boc)₂O], benzyl chloroformate, 9-fluorenylmethyloxycarbonylchloride (FmocCl), 1,8-diazabicyclo[5.4.0]undec-7-ene (DBU) and N-methylmorpholine were supplied by ACROS (Geel, Belgium). 1-hydroxy-7-azabenzotriazole (HOAt) and its tetramethyluronium derivative (HATU) were purchased from Perspective Biosystems. [2-(1H-bentotriazole-1-yl)-1,1,3,3-tetramethyluronium hexafluorophosphate] (HBTU) from Advanced Chemtech. Benzotriazole-1-yl-oxy-tris-pyrrolidino-phosphonium hexafluorophosphate (PyBOP®) was obtained from Advanced Chemtech. Diethyl azodicarboxylate (DEAD) and sodium hydride (NaH) were purchased from Sigma-Aldrich. P-[(R,S)-a-[1-(9H-fluoren-9-yl)-methoxy-formamido]-2,4-dimethoxybenzyl]-phenoxyacetic acid (Fmoc-Rink linker) was supplied by Novabiochem. Lipofectamine 2000® was obtained from Invitrogen. PBS buffer (pH7.4) was obtained from Sigma. Oligonucleotide assembly was performed on an Expedite DNA synthesizer (Applied Biosystems) using phosphoramidites from Glen Research.

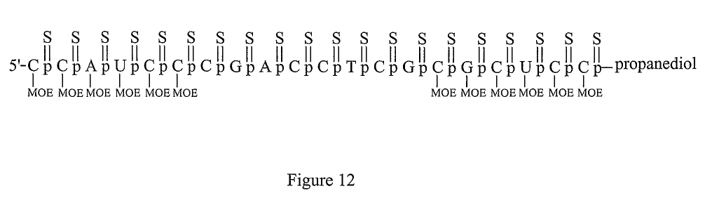

The 11 and the 18-oligonucleotide sense sequences without cholesterol, as well as the tetramethyl rhodamine (TMR)-labeled oligonucleotide antisense sequence, complementary to the gene encoding the P-glycoprotein (Astriab-Fisher, A. et al. (2000) Biochem. Pharmacol. 60, 83-90), were obtained from Invitrogen. The phosphorothioate oligonucleotide from FIG. 12 (SEQ ID NO: 23) is a gift from ISIS.

Cholesterylacetaldehyde diethylacetal (2). To a solution of cholesterol (5 g, 12.9 mmol, 1 eq.) in a mixture of anhydrous THF/DMF (1:1, 200 ml), NaH (3.2 g, 77.6 mmol, 6 eq.) and, after 5 min, bromoacetaldehyde diethylacetal (6.3 ml, 38.8 mmol, 3 eq.) were added in anhydrous circumstances. A nitrogen-flushed reflux condensor was attached and the mixture was heated at reflux for 18 h. The excess NaH was decomposed using MeOH/CH₂Cl₂ and mild shaking After evaporation to dryness, silicagel based chromatographic purification was performed with a stepwise gradient of a hexane/EtOAc mixture (98:2 to 95:5) as eluent, yielding 4.3 g (8.56 mmol, 66.3%) of the title compound. ¹³C NMR: δ=140.97 (C-3), 121.58 (C-4), 101.46 (β-CH), 79.73 (C-1), 68.77 (α-CH₂), 62.09 (Et, CH₂O), 56.72 (C-7), 56.12 (C-10), 50.11 (C-14), 42.25 (C-11), 39.73; 39.45; 38.97 (C-2; C-12; C-24), 37.15 (C-16), 36.78 (C-15), 36.12 (C-22), 35.72 (C-20), 31.84 (C-5, C-6), 28.26; 28.13; 27.92 (C-17; C-9; C-25), 24.19; 23.73 (C-8; C-23), 22.73; 22.46 (C-26; C-27), 20.97 (C-13), 19.27 (C-18), 18.60 (C-21), 15.27 (Et, CH₃), 11.75 (C-19). Mass calcd. for [M+Cl]— C₃₃H₅₈O₃Cl₁: 537.4074, found: (ESI−): 537.4094.

Cholesterylacetaldehyde (3). To a solution of 2 (4.1 g, 8.17 mmol) in CH₂Cl₂ (80 ml), 20 ml TFA/H₂O (1:1) was added and the reaction mixture was stirred for 4.5 h at room temperature (complete conversion). NaOH 1 N was used to neutralise the solution and subsequently an extraction with CH₂Cl₂ was performed. The organic layer was dried over anhydrous MgSO₄, filtered and evaporated in vacuo to dryness, yielding 3.45 g (8.07 mmol, 98%) of the title compound. ¹³C NMR: δ=201.70 (β-CH), 140.30 (C-3), 122.22 (C-4), 80.37 (C-1), 73.81 (α-CH₂), 56.69 (C-7), 56.12 (C-10), 50.08 (C-14), 42.25 (C-11), 39.70; 39.42; 38.76 (C-2; C-12; C-24), 36.97 (C-16), 36.72 (C-15), 36.12 (C-22), 35.69 (C-20), 31.81 (C-5, C-6), 28.10; 27.92 (C-17; C-9; C-25), 24.19; 23.73 (C-8; C-23), 22.70; 22.46 (C-26; C-27), 20.97 (C-13), 19.24 (C-18), 18.60 (C-21), 11.75 (C-19). Mass calcd. for [M]⁺C₂₉H₄₈O₂: 428.3654, found (El+): 428.3647

Cholesterylacetic acid (4). Compound 3 (3 g, 7.0 mmol) was dissolved in acetone (105 ml) and 1.5 ml Jones' reagent (26.72 g CrO₃/23 ml conc. H₂SO₄/H₂O to 100 ml) was added dropwise while the reaction mixture was cooled on ice. If necessary (TLC), more Jones' reagent was added to completely convert 3 into its carboxylic acid. After 10 min., acetone and MeOH were added, the mixture was filtered and the remaining filtrate was evaporated to dryness. The residual solid was purified by column chromatography on silica gel by elution with Hexane/EtoAc/AcOH (8:2:0.1), yielding 2.1 g (4.76 mmol; 68%) of the title compound. ¹³C NMR: δ=173.54 (COOH), 140.00 (C-3), 122.46 (C-4), 80.46 (C-1), 65.16 (α-CH₂), 56.69 (C-7), 56.12 (C-10), 50.05 (C-14), 42.25 (C-11), 39.67; 39.45; 38.61 (C-2; C-12; C-24), 36.90 (C-16), 36.69 (C-15), 36.12 (C-22), 35.69 (C-20), 31.78 (C-5, C-6), 28.13; 28.01; 27.92 (C-17; C-9; C-25), 24.19; 23.73 (C-8; C-23), 22.73; 22.46 (C-26; C-27), 20.97 (C-13), 19.24 (C-18), 18.60 (C-21), 11.75 (C-19). Mass calcd. for [M]⁺ C₂₉H₄₈O₃: 444.3603, found (El−): 444.3606

Nα-Fmoc-Nε-cholesterylacetyl-lysine-O-Me (5). Compound 4 (1.7 g, 3.7 mmol, 1 eq.) and HATU (1.35 g, 4.07 mmol, 1.1 eq.) were suspended in 85 ml dry DMF. To this suspension, DIEA (1.3 ml, 7.4 mmol, 2 eq.) was added and the resulting solution was stirred for 2 h at room temperature. To this mixture, a solution of Fmoc-Lysine-OMe.HCl (1.7 g, 4.07 mmol, 1.1 eq.) and DIEA (1.3 ml, 7.4 mmol, 2 eq.) in 15 ml dry DMF was added and stirring was continued at room temperature for 18 h. After evaporation the residue was dissolved in EtOAc and the organic phase was washed with HCl (1N), water, saturated Na₂CO₃ solution, brine and water. The organic layer was dried over sodium sulfate, filtered an evaporated. The remaining oil was purified by chromatography using a mixture of CH₂Cl₂/MeOH (99:1) as eluent. Evaporation of the pooled fractions yielded 2.5 g (3.1 mmol, 84%) of compound 5. ¹³C NMR: δ=172.66 (Lys, COOMe), 170.41 (CONH); 156.51 (OCONH), 144.04 (Fmoc, C-10/11), 141.73 (Fmoc, C-12/13), 140.12 (Chol, C-3), 127.68 (Fmoc, C-3/6), 127.04 (Fmoc, C-2/7), 125.07 (Fmoc, C-1/8), 122.28 (Chol, C-4), 119.97 (Fmoc, C-4/5), 80.39 (Chol, C-1), 67.47 (Fmoc, CH₂O), 66.52 (Chol, α-CH₂), 56.66 (Chol, C-7), 56.08 (Chol, C-10), 52.38 (Lys, CH₃O), 51.14 (Lys, α-CH) 50.01 (Chol, C-14), 47.22 (Fmoc, C-9), 42.24 (Chol, C-11), 40.61 (Lys, ε-CH₂NH), 39.67; 39.45; 38.82 (Chol, C-2; C-12; C-24), 36.93 (Chol, C-16), 36.69 (Chol, C-15), 36.12 (Chol, C-22), 35.69 (Chol, C-20), 32.26 (β-CH₂(Lys)), 31.80 (Chol, C-5, C-6), 29.62 (δ-CH₂(Lys)), 28.13; 28.01; 27.92 (Chol, C-17; C-9; C-25), 24.19; 23.73 (Chol, C-8; C-23), 22.73; 22.46 (Chol, C-26; C-27), 22.34 (γ-CH₂(Lys)) 20.97 (Chol, C-13), 19.27 (Chol, C-18), 18.63 (Chol, C-21), 11.74 (Chol, C-19). Mass calcd. for [M+Cl]⁻C₅₁H₇₂N₂O₆Cl₁: 843.5078, found: (ESI−): 843.5049.

Nα-Fmoc-Nε-cholesterylacetyl-lysine (6). The cleavage of the methyl ester was carried as described in literature (Qabar, M. N. et al. (1997) Tetrahedron 53, 11171-11178.). To compound 5 (1.5 g, 1.8 mmol, 1 eq.) in 20 ml THF at 0° C. was added 0.2 N LiOH (18 ml, 3.6 mmol, 2 eq.) and the reaction was stirred for 40 min. To the reaction mixture, 50 ml of a biphase of 0.2 N HCl/EtOAc (1:1) was added, the aqueous layer was extracted three times with EtOAc and the combined organic layers were dried over MgSO₄, filtered and evaporated to dryness. The remaining product was purified by column chromatography on silica gel with a CH₂Cl₂/MeOH-mixture (95:5) as eluent, yielding 1.1 g (1.4 mmol, 77%) of the title compound. ¹³C NMR: δ=174.78 (Lys, COOH), 171.14 (CONH); 156.51 (OCONH), 144.01 (Fmoc, C-10/11), 141.37 (Fmoc, C-12/13), 140.06 (Chol, C-3), 127.71 (Fmoc, C-3/6), 127.07 (Fmoc, C-2/7), 125.10 (Fmoc, C-1/8), 122.31 (Chol, C-4), 120.00 (Fmoc, C-4/5), 80.46 (Chol, C-1), 67.31 (Fmoc, CH₂O), 66.52 (Chol, α-CH₂), 56.63 (Chol, C-7), 56.08 (Chol, C-10), 51.47 (Lys, α-CH) 49.98 (Chol, C-14), 47.19 (Fmoc, C-9), 42.24 (Chol, C-11), 40.58 (Lys, ε-CH₂NH ), 39.67; 39.45; 38.82 (Chol, C-2; C-12; C-24), 36.90 (Chol, C-16), 36.69 (Chol, C-15), 36.12 (Chol, C-22), 35.72 (Chol, C-20), 32.26 (β-CH₂(Lys)), 31.80 (Chol, C-5, C-6), 29.62, 29.19 (δ-CH₂(Lys)), 28.16; 28.01; 27.92 (Chol, C-17; C-9; C-25), 24.19; 23.76 (Chol, C-8; C-23), 22.73; 22.46 (Chol, C-26; C-27), 22.34 (γ-CH₂(Lys)), 20.97 (Chol, C-13), 19.24 (Chol, C-18), 18.63 (Chol, C-21), 11.74 (Chol, C-19). Mass calcd. for [M−H]⁻ C₅₀H₆₉N₂O₆: 793.5155, found: (ESI−): 793.5143.

Solid Phase Synthesis.

A. Peptide synthesis. Synthesis of the conjugates was performed on the LCAA-CPG carrier. For each amino acid or compound to be coupled, different reaction conditions were applied. After every reaction, the solution was filtered and the beads were washed with DMF (2 times), CH₂Cl₂ (2 times), and acetonitrile (2 times). The beads were dried and Fmoc-or MMTr-substitution levels were determined. Hereto, an accurately measured quantity of resin was suspended in exactly 25 mL of 20% piperidine in DMF or in a HClO₄/EtOH mixture (3:2), respectively. After 15 min. the absorbance was measured at 300 nm for fulvene (or adduct) determination and at 475 nm for methoxytrityl cation detection (ε_{Fmoc}=7500, ε_{MMTr}=55000). If the coupling yields were unsatisfactory, a second coupling reaction was performed. Before each new coupling, the remaining amino groups were capped using 1.5 ml of 20% acetic anhydride in THF and 1.5 ml of 20% 1-methylimidazole and 40% 2,4,6-trimethylpyridine in THF for 15 min., and washed with acetonitrile and DMF. Fmoc-deprotection was carried out with a piperidine/DMF mixture (2:8 v:v, 2 mL) for 5 min. and washing for 15 min., while for MMTr-deprotection, 3% TCA in CH₂Cl₂ was used 3 to 4 times for 3 min., until no yellow color was detectable.

Coupling of Fmoc-sarcosine to LCM-CPG was generally performed as described in literature (Stetsenko, D. A. et al. (2001) Bioconjugate Chem. 12, 576-586.). To a solution of Fmoc-sarcosine (116 mg, 0.37 mmol, 4 eq.) and HOBt (31 mg, 2.23 mmol, 2.5 eq.) and HBTU (88 mg, 0.23 mmol, 2.5 eq.) in 2 ml DMF, DIEA (78 μL, 0.46 mmol, 5 eq.) was added and the mixture was shaken for 5 min. More DIEA (78 μL, 0.46 mmol, 5 eq.) was added and the solution was then poured onto LCAA-CPG beads (1 g, 92 μmol, 1 eq.). The reaction mixture was occasionally shaken for 2 h. Coupling of MMTr-butyric acid was obtained by adding a solution of 4-OMMTr-butyric acid (Hovinen, J. et al. (1994) Tetrahedron 50, 7203-7218.). (220 mg, 0.46 mmol, 5 eq.), HOBT/HBTU (88 mg, 0.23 mmol, 2.5 eq.) and DIEA (158 μL, 0.92 mmol, 10 eq.) in 2 mL DMF. Following deprotection of MMTr, Fmoc-glycine was coupled to the 4-OH group of 4-OH-butyric acid by occasionally swirling the beads with a reaction mixture of Fmoc-glycine (273 mg, 0.92 mmol, 10 eq.), DIC (72 μL, 0.46 mmol, 5 eq.) and DMAP (17 mg, 13.8 μmol, 0.15 eq.) in DMF (2 ml).

The cholesterylacetamide 6 was attached to glycine by using a solution of 6 (0.29 mg, 0.37 mmol, 4 eq.), HBTU (140 mg, 0.37 mmol, 4 eq.), HOBt (49 mg, 0.37 mmol, 4 eq.) and DIEA (168 μL, 0.92 mmol, 10 eq.) in 1 mL DMF. The mixture was shaken for 5 min. before it was added to the Fmoc-deprotected support (yield: 90%).

For single oligonucleotide loading, 4-O-MMTr-butyric acid was coupled as a linker before starting oligonucleotide synthesis. For the branching of purposes, Nα-Fmoc-Nε-Fmoc-lysine (4 eq.) was used. The protected lysine was attached to the support by adding a solution of HBTU (4 eq.), HOBt (4 eq.) and DIEA (10 eq.) in DMF. Subsequently, 4-O-MMTr-butyric acid was coupled. The amount of 4-O-MMTr-butyric acid used was always 4 times the amount of free NH₂-groups available for coupling.

B. Oligonucleotide synthesis. Four different supports were used for assembly of the undeca-and octadecamers. The number of hydroxyl groups per gram of support was determined by detritylation and amounted to 48, 80 and 130 μmol/g for the supports 9, 11, 13 respectively. The assembly of oligonucleotides was done on the Expedite following a standard 1 μmol protocol, using only 0.5 μmol of support and a 4 min. coupling time. Following deprotection with AMA (methylamine-ammonia 1:1) for 2 h at 25° C., the products were purified by gel filtration using 30% aqueous acetonitrile to avoid adsorption of the cholesteryl moieties to the support. Depending whether a single, double or four chains of oligonucleotides needed to be coupled to the cholesterol moiety, one of the different supports 9, 11, 13 were used. The conjugates could be purified by RP-HPLC on PLRP-S (100 Å, 8 μM, 250×7 mm, Polymer Laboratories) with a CH₃CN gradient in 0.05 M ammonium acetate, pH 7.0. In general, in ascending order, tetrameric oligo containing dendrimer eluted before the trimer, dimer and monomer species. Analysis was accomplished by HPLC/MS on a capillary chromatograph (CapLC, Waters, Milford, Cailf.). Columns of 150 mm×0.3 mm length (LCPackings, San Francisco, Calif.) were used. Oligonucleotides were eluted with a triethylammonium-1,1,1,3,3,3-hexafluoro-2-propanol: acetonitrile solvent system, and the flow rate was 5 μl/min. Electrospray spectra were acquired on a quadrupole orthogonal acceleration/time-of-flight mass spectrometer (Q-Tof-2, Micromass, Manchester, U.K.) in negative ion mode. The scan time used was 2 seconds. The combined spectra from a chromatographic peak were deconvoluted using the MaxEnt algorithm of the software Masslynx 3.4 (Micromass). Theoretical oligonucleotide masses were calculated using the monoisotopic element masses for masses up to 8000 and using the average element masses for masses above 8000. The data are given in Table 1.

3.2 Results of the Synthesis of the Cholesterol-Bioconiugate Dendrimer

For the synthesis of the monomeric, dimeric and tetrameric cholesterol oligonucleotide conjugates, a strategy was used based on lysine chemistry. Lysine has an α-and ε-amino group and binding of one lysine unit via its α carboxyl function to a solid support allows to liberate two functional amino groups that can be used for oligonucleotide synthesis.

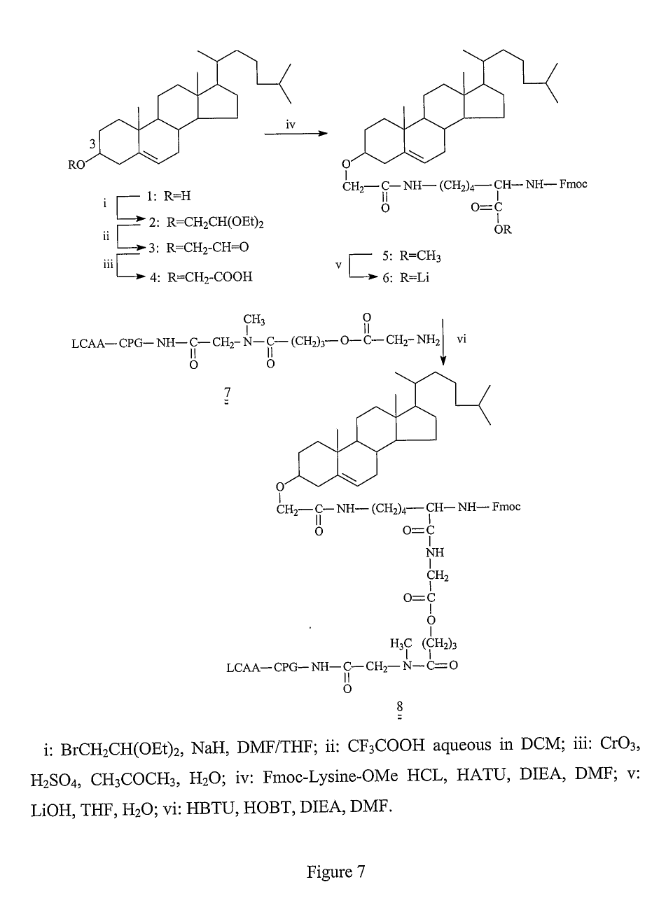

Cholesterol (1) was derivatized at its C-3 hydroxyl group with a functional group that is stable against acid and base and that can be used for the formation of an amide bond (FIG. 7). Reaction of cholesterol with chloroacetic acid in the presence of NaH gives poor yields. Therefore cholesterol was first reacted with the diethylacetal of bromoacetaldehyde in the presence of NaH in a mixture of THF/DMF for 18 h at 85° C. In this way cholesterylacetaldehyde diethylacetal (2) was obtained in 70% yield. Reaction of 2 with Jones' reagent resulted in the formation of the desired cholesterylacetic acid 4, but the reaction went to completion only after 24 h, and multiple side products were formed. In order to avoid side reactions, the acetal was first hydrolysed with trifluoroacetic acid in dichloromethane for 4 h, giving the aldehyde 3. The oxidation of the aldehyde to the carboxylic acid with Jones' reagent was completed after 10 min., giving a clean reaction product. The oxidation using hypochlorite was less effective.

Subsequently, Fmoc-Lysine-OMe was coupled with its free ε-NH₂-group to cholesteryl acetic acid using HATU, DIEA and TEA (FIG. 7). Compound 5 was then deprotected by hydrolysing the methyl ester with LiOH 0.2 N in THF/H₂O. The carboxylic acid of 6 could then be used to couple 6 to the solid support 7. This solid support material was obtained by derivatization of LCAA-CPG with sarcosine. Following, MMTr-O-butyric acid was attached to the sarcosine and detritylated with trifluoroacetic acid. The free hydroxyl group was used for coupling with Fmoc-glycine with formation of a base labile ester bond. After Fmoc deprotection, compound 6 was coupled to the solid support 7 using a mixture of HBTU, HOBT and DIEA (FIG. 7). In this way, a cholesterol derivatized lysine connected to a solid support via an ester bond was obtained, as general support for the synthesis of the desired oligonucleotide monomers (10a,b) and dendrimers (12a,b and 14a,b).

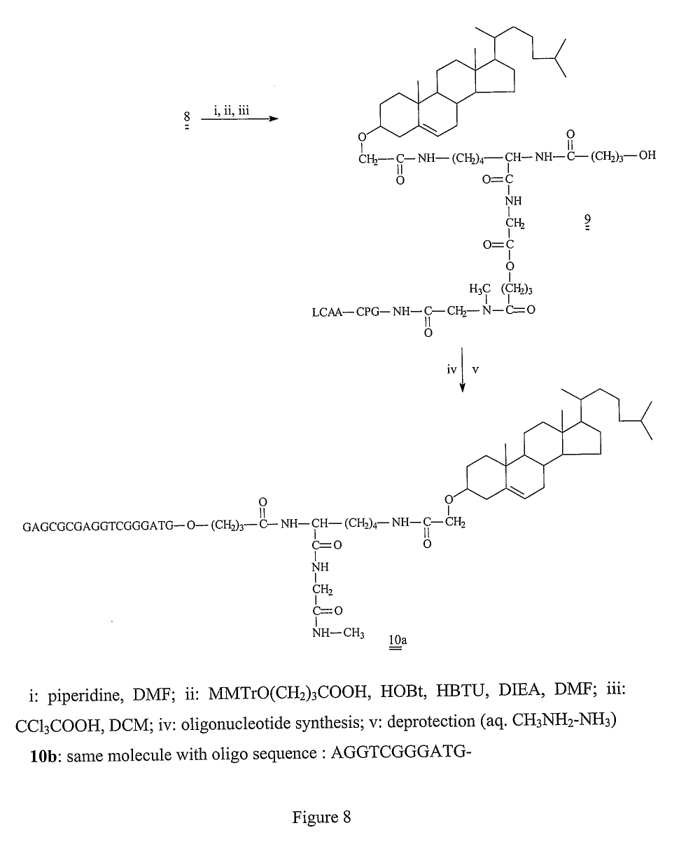

The synthesis of the cholesteryl-lysyl oligonucleotide (monomer 10a,b) is shown in FIG. 8. After Fmoc deprotection of 8, coupling of MMTr-O-butyric acid and MMTr deprotection, the solid material 8 was used for oligonucleotide synthesis with the phosphoramidite approach. An 11-mer and 18-mer were assembled with a sequence complementary to the antisense oligonucleotide.

Deprotection using AMA reagent (conc. aq. ammonia-aq. methylamine 1:1) yielded 10a,b. The conjugate was concomitantly removed from the solid support by cleavage of the labile ester group with formation of the methylamide (with 5% amide formation according to MS analysis).

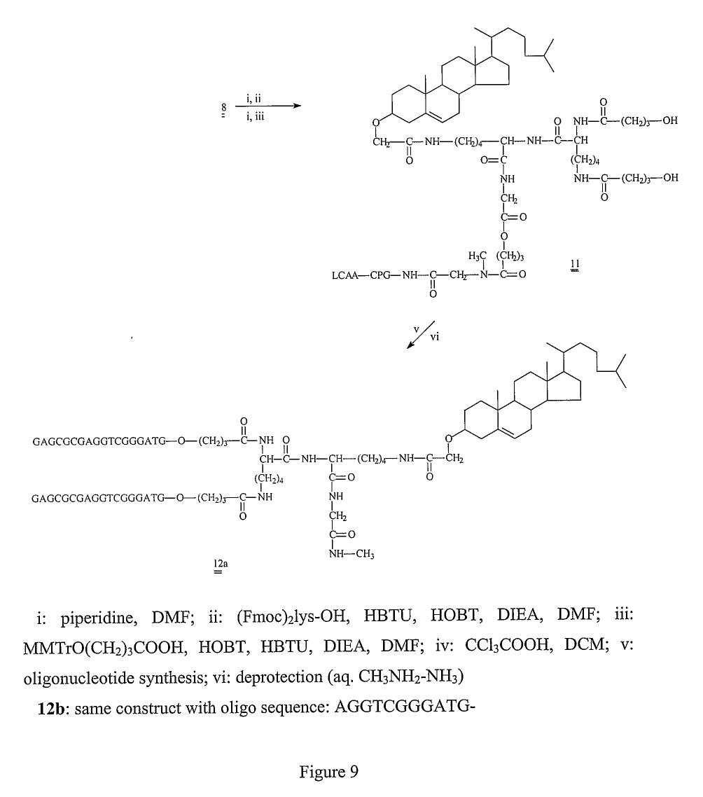

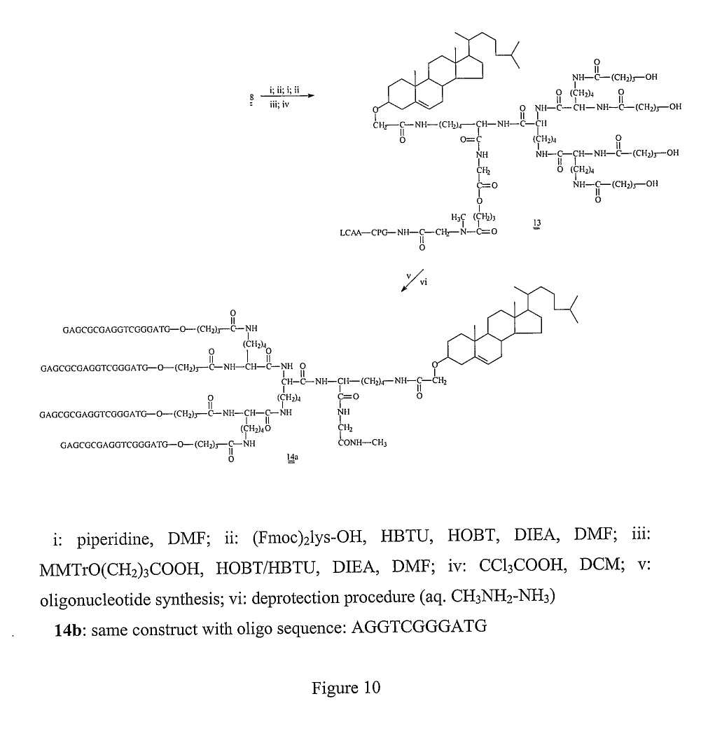

Branching of the peptide linker was carried out by coupling of Fmoc-protected lys-OH on deprotected 8 (FIG. 9). After a second deprotection step and binding of hydroxybutyric acid, the solid support can be used for the synthesis of two oligonucleotides on the same cholesteryl derivatized peptide (FIG. 9). Repetition of the protocol of coupling Fmoc-protected lys-OH, Fmoc deprotection, and coupling of hydroxybutyric acid might lead to an exponential branching of the cholesterol derivatized solid support, enabling the assembly of multiple oligonucleotides on one cholesterol moiety. This is demonstrated with the branched dendrimer 14a,b in FIG. 10. However, mass spectral analysis of the major fraction shows that only three of the four hydroxybutyryl moieties are used for oligonucleotide synthesis. The HPLC sample of the trimeric compound shows only a small fraction of the four-branched dendrimer. The three-branched molecule has an acetylated lysine moiety (although it is not clear if acetylation has occurred at the α-N or the ε-N of lysine). Apparently, steric hindrance or the occurrence of a side reaction hampers the formation of the four-branched construct. We were able, however, to isolate sufficient amounts of the tetrameric construct by chromatographic purification to carry out biological tests.

TABLE 2
Results of Mass Spectrometry Analysis of the Monomeric (10),
Dimeric (12) and Tetrameric (14) Constructs

| | |
|---|---|
| a series | b series |

| | Calcd. | Found⁽³⁾ | Calcd. | Found⁽³⁾ |
|---|---|---|---|---|
| Monomer | 6433.4⁽¹⁾ | 6432.8 | 4226.1⁽¹⁾ | 4225.8 |
| Dimer | 12358.4⁽²⁾ | 12356.4 | 7937.8⁽¹⁾ | 7937.2 |
| Tetramer | 24202.6⁽²⁾ | ND | 15368.9⁽²⁾ | 15367.0 |

⁽¹⁾monoisotopic mass
⁽²⁾average mass
⁽³⁾mass obtained after deconvolution of the electrospray spectrum
ND: not determined

Using these three different supports, cholesterol oligonucleotide complexes were formed in a ratio of 1:1 (10a,b), 1:2 (12a,b) and 1:4 (14a,b) and the obtained products were used for hybridization with the antisense oligonucleotide followed by cationic liposome formulation. Although it is difficult to analyse the way the antisense oligonucleotides are associated to the sense-dendrimers, the stability of the complex was determined using melting experiments. In all cases, the melting point was above 70° C., demonstrating that stable complexes are formed.

### 3.3 Experimental Procedure used for the Investigation of the Cholesterol Bioconiugate Dendrimer

Procedures used for evaluation of the delivery of oligomeric compounds and their activity (i.e. cells, treatment with dendrimeric bioconjugate compositions or oligomeric compounds and evaluation of the activity) were performed according to the general procedures described herein.

### 3.4 Biological Activity

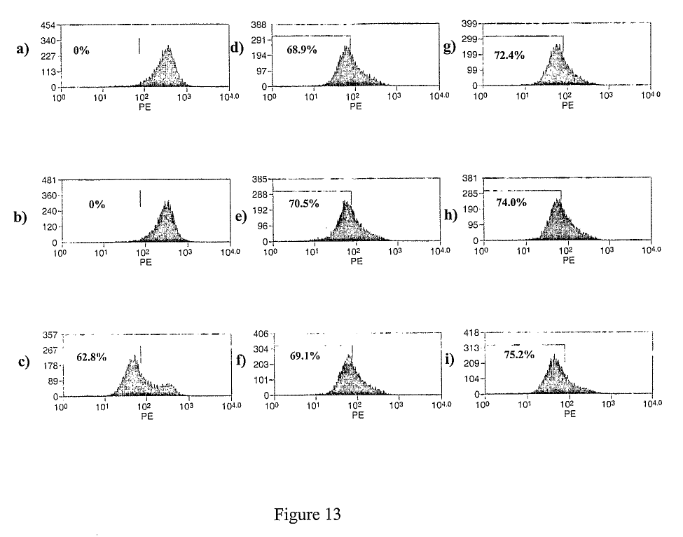

The biological activity was determined using the 2′-MOE gapmer of phosphorothioate as the antisense strand (FIG. 12). The concentration was determined spectroscopically. When incubating the antisense oligonucleotide with Lipofectamine 2000, a maximum activity was obtained at 50 nM (giving 42.2% P-gp shift). Therefore this antisense concentration was used in further experiments. When the cholesterol dendrimers 12a and 14a were tested in complex with the antisense construct without adding lipofectamine 2000, a significant effect was seen after a long incubation time (72 h). This means that the dendrimers alone were not very effective in delivering the antisense oligonucleotide. Significant effects were obtained when the dendrimers 12a and 14a were used in the presence of lipofectamine 2000 (Table 3-FIG. 13). After 48 h incubation the antisense oligonucleotide gives 62.8% P-gp shift, while in the presence of the dendrimers and using 1:1, 1:2 or 1:4 molar ratios of dendrimer and antisense oligonucleotides (fixed at 50 nM), the P-gp shift varied from 68.9% to 74.0%. The biological activity is somewhat increased when the antisense oligonucleotides are more condensed (ratio 1:1 versus ratio 1:2 or 1:4).

TABLE 3
Percent Reduction of P-glycoprotein Expression Level in
Mouse 3T3 Cells by Dendrimer-antisense Complexes

| | | | Tetramer | | Tetramer |
|---|---|---|---|---|---|
| | | Dimer 12a + | 14a + | Dimer 12a + | 14a + |
| | Antisense | antisense | antisense | antisense | antisense |
| | alone | ratio 1:1 | ratio 1:1 | ratio 1:2 | ratio 1:4 |
| P-gp | 62.8% | 68.9% | 70.5% | 72.4% | 74.0% |

NIH3T₃-MDR cells were transfected with the complexes of 12a or 14a, the antisense oligonucleotide and Lipofectamine 2000. Results were obtained after 48 h incubation in 5% FBS. The ratio between 12a or 14a and the antisense oligonucleotide was 1:1, 1:2 and 1:4. The antisense oligonucleotide was always used at 50 nM concentration.

### Example 4

### Delivery of Oligonucleotides with the Tat-PAMAM Dendrimeric Bioconjugate

PAMAM dendrimers are cationic polymers that have already been used for the delivery of genes and oligonucleotides to cells. We wanted to investigate the effect of the coupling of a peptide to the PAMAM dendrimer on the delivery of oligonucleotides (single-stranded and double-stranded). The delivery of siRNA with PAMAM dendrimers was also investigated. The procedures used for these experiments were as described hereinabove.

### 4.1 Preparation of the Tat-PAMAM Dendrimeric Bioconjugate

Synthesis of Tat-peptide: Tat peptide with a cysteine residue at C-terminus (NH₂-RKKRRQRRRPPQGGC-COOH) was synthesized at the UNC Microprotein Sequencing & Peptide Synthesis Facility or purchased from Cell Essentials (Boston, Mass.). Phosphorothioate oligonucleotide was purchased from Midland Certified Reagents Company (Midland, Tex.). SiRNA was purchased from Dharmacon (Lafayette, Colo.). PAMAM G5 dendrimer was purchased from Dendritech Inc. (Midland, Mich.). BODIPY FL sulfosuccinimidyl ester and Thiol and Sulfide quantitation kit were purchased from Molecular Probes (Eugene, Oreg.). Sulfo-LC-SMPT, a cross-linking reagent, was purchased from Pierce (Rockland, Ill.).

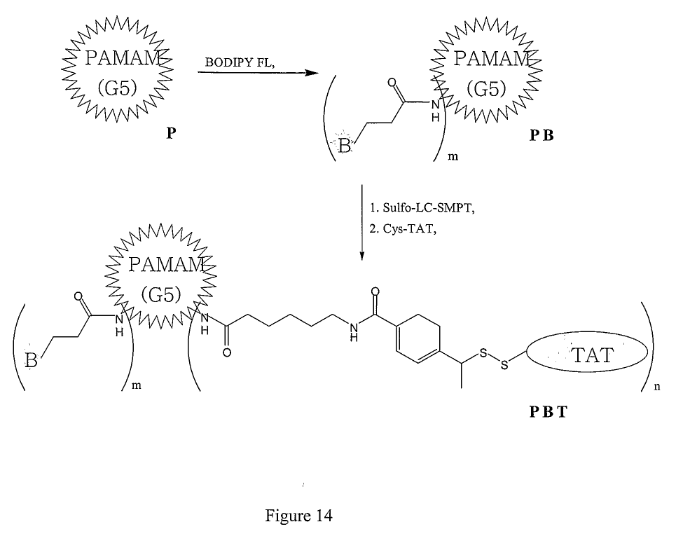

Preparation of PAMAM coniugate of Tat peptide (BPT) (see FIG. 14): PAMAM G5 dendrimer (24.32 mg, 0.844 μmole) was reacted with BODIPY FL sulfosuccinimidyl ester (2 mg, 4.219 μmole) in 100 mM NaHCO₃ for 12 hours at 4 C. The reaction was then purified on a G-25 size exclusion column to remove remaining free BODIPY derivatives. The large molecular weight product recovered from G-25 was dried under reduced pressure to give 82% yield. The product was then analyzed by spectrophotometry for BODIPY absorbance (λₘₐₓ=502 nm) to give average BODIPY/PAMAM ratio of 1.8. The BODIPY-PAMAM conjugate (BP, 10 mg, 0.343 μmole) was then reacted with sulfo-LC-SMPT (4 mg, 6.63 μmole) in PBS for 12 hours at 4° C., and purified on G-25 column. Tat peptide (25 mg, 13.30 μmole) in PBS/50 mM EDTA (pH 7.4) was then added to the intermediate, and the reaction was kept at room temperature for 24 hours. The ratio of Tat/PAMAM on the reaction product was determined as 15.9 by the release of pyridine-2-thione (λₘₐₓ=343 nm) by spectrophotometry. The reaction was purified on G-25 column to yield 68%. The product (BPT) was subjected to thiol and sulfide quantitation experiments according to the manufacturer's recommendation.

### 4.2 Antisense and siRNA Targeted to the MDR1 Gene and Scrambled/mismatched Controls Used for Testing the (Tat)-PAMAM Dendrimeric Delivery

| | | | |
|---|---|---|---|
| Name | Target | Sequence | Chemical property |

| | | | |
|---|---|---|---|
| 5995 | AUG start | 5′-CCA TCC CGA CCT CGC GCT CC-3′ | Phosphorothioate |
| | codon | | antisense |
| | of MDR1 | | |
| Cy5-5995 | AUG start | 5′-(Cy5)CCA TCC CGA CCT CGC GCT CC-3′ | Phosphorothioate |
| | codon | | antisense |
| | of MDR1 | | |
| 10221 | Scrambled | 5′-CAC CAC CCC CCT CGC TGG TC-3′ | Phosphorothioate |
| | control | | antisense |
| | of 5995 | | |
| ORF1 | Targets | 5′-GUAUUGACAGCUAUUCGAAdTdT-3′ | siRNA |
| | 1545-1565 | 3′-dTdTCAUAACUGUCGAUAAGCUU-5′ | |
| | of MDR1 | | |
| Cy5-ORF1 | Targets | 5′-(Cy5)GUAUUGACAGCUAUUCGAAdTdT-3′ | siRNA |
| | 1545-1565 | 3′-dTdTCAUAACUGUCGAUAAGCUU-5′ | |
| | of MDR1 | | |
| Nischarin | Nischarin | 5′-CCUCGUGCACCUUGACCUGdTdT-3′ | siRNA |
| | targeting: | 3′-dTdTGGAGCACGUGGAACUGGAC-5′ | |
| | control | | |
| | for ORF1 | | |

### 4.3 Oligonucleotide Treatment

PAMAM G5 dendrimer (P), PAMAM G5 conjugated to BODIPY (BP), BP conjugate of Tat peptide (BPT) or Lipofectamine 2000 were complexed to antisense or siRNA in Opti-MEM medium 20 minutes before cell treatment. After 24 hours of cell seeding onto 6 well plates, cells were treated with the freshly prepared antisense or siRNA complexes with P, BP, BPT or Lipofectamine 2000 in DMEM medium (2 ml) containing 10% FBS for 4 hours at 37° C. The cells were then washed twice with 10% FBS/DMEM and incubated in the same medium at 37° C. After 1 hour, cells were washed again with DMEM/10% FBS and further incubated in DMEM/2% FBS for 64 hours.

### 4.4 Immunostaining of P-glycoprotein

The cell surface expression of P-glycoprotein in viable cells was studied by immunostaining and flow cytometry . After treating NIH 3T3 MDR cells with antisense or siRNA and further incubating them 64 hours as described above, cells were trypsinized, washed twice with PBS, counted for normalization and incubated with R-phycoerythrin conjugated mouse anti-human p-glycoprotein (20 μl, BD Biosciences, San Diego, Calif.) in 50 μl PBS at 4° C. After 45 minutes, cells were washed once with PBS/10% FBS and twice with PBS. The levels of R-phycoerythrin immunostaining on viable cells (identified by light scattering) were then quantified on a Becton Dickinson flow cytometer with Cicero software (Cytomation, Fort Collins, Colo.).

4.5 Results of siRNA Delivery

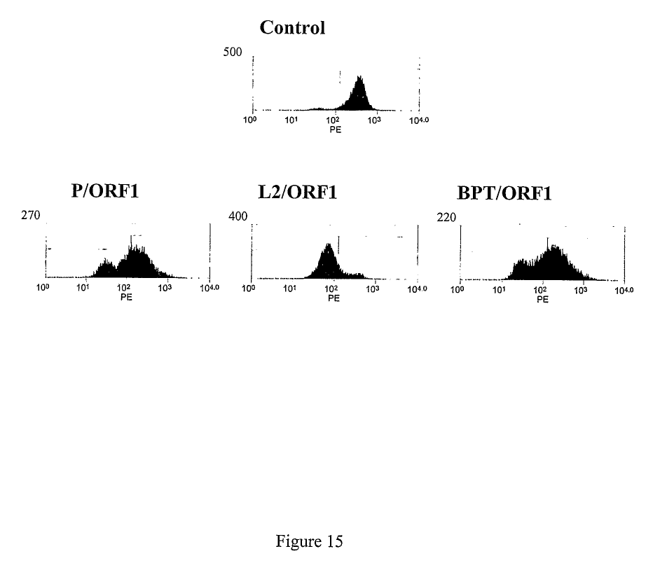

As seen in FIG. 15, the PAMAM dendrimer was able to deliver siRNA which resulted in a reduction of P-glycoprotein expression in the NIH 3T3 MDR cells as monitored by immunostaining and flow cytometery. The effect of the dendrimer was not as high as for an optimized dose of Lipofectamine 2000, but nonetheless was effective. The conjugation of the dendrimer with TAT peptide provided a further increment in effectiveness as judged by the leftward shift in the immunostaining profile. Control siRNA had no effect (not shown). We can thus conclude that positively charged PAMAM dendrimers are able to deliver both siRNA oligonucleotides into cells and these oligonucleotides can then down-regulate expression of the target gene. Conjugation of the PAMAM dendrimer provides a further enhancement for siRNA delivery.

### 4.6 Results of Antisense Oligonucleotide Delivery

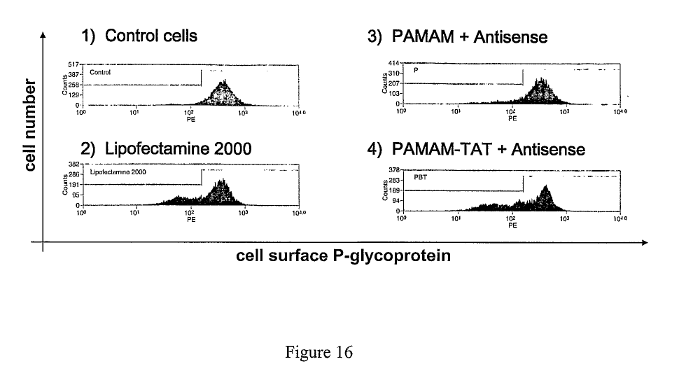

As seen in FIG. 16, the PAMAM dendrimer was able to deliver antisense oligonucleotide that resulted in a reduction in P-glycoprotein expression in the NIH 3T3 MDR cells as monitored by immunostaining and flow cytometery. The PAMAM dendrimer was also somewhat less effective than an optimized dose of Lipofectamine 2000. The conjugation of the dendrimer with TAT peptide provided an increment in effectiveness as judged by the leftward shift in the immunostaining profile. Control antisense had no effect (not shown). Positively charged PAMAM dendrimers are therefore able to deliver antisense oligonucleotides into cells and these oligonucleotides can then down-regulate expression of the target gene. Conjugation of the PAMAM dendrimer provides a larger enhancement for antisense delivery than for siRNA delivery.

None of the PAMAM dendrimer formulations were as effective as the cationic lipid Lipofectamine 2000. However, the dendrimer complexes are smaller in size than cationic lipid complexes and could offer advantages for in vivo delivery since they will not occlude the capillaries.

### 4.7. Delivery of Antisense Via Hybridization Complexes

The Tat-PAMAM dendrimeric bioconjugate is used for the delivery of hybridization complexes of the presently disclosed subject matter in order to evaluate the delivery hereof by using a dendrimeric bioconjugate.

Therefore, duplexes as described herein, namely the gapmers of example 2 are added to the Tat-PAMAM dendrimeric bioconjugate, are complexed to the hybridization complex as described herein above and cells are treated.

Experiments for the evaluation of the activity are performed as described herein above.

### Example 5

### Delivery with a Tat-Dendrimeric Oligonucleotide Bioconiugate

As another example of the dendrimeric bioconjugate, a conjugate of Tat-peptide with an oligonucleotidic dendrimer is prepared. The procedure that is used comprises the following: (i) a protected Tat-peptide is prepared using standard peptide chemistry with protected amino acids, (ii) coupling of Fmoc-lysine-OMe or an analoguous structure comprising a protected amino group and a protected carboxylgroup which are separately cleavable, (iii) deprotection of the carboxylgroup, (iv) coupling the obtained compound to a solid phase through an acid labile coupling, like an ester, (v) coupling a linker with a free hydroxyl as reactive group and (vi) perform standard oligonucleotide synthesis followed by cleaving the compound of from the solid phase. For obtaining a dendrimer, step (iv) is first followed by (vi a) which comprises the coupling of a compound with two protected amino functions and a free carboxylgroup like (Fmoc)₂-Lys-OH.

Experiments for the evaluation of the activity are performed as described herein above.

It will be understood that various details of the described subject matter can be changed without departing from the scope of the described subject matter. Furthermore, the foregoing description is for the purpose of illustration only, and not for the purpose of limitation.

What is claimed is:

1. A method of inhibiting the expression of a gene in a cell, the method comprising administering to the cell a composition comprising a hybridization complex, wherein the hybridization complex comprises:
(a) a first oligonucleotide strand, wherein the first oligonucleotide strand comprises a fully or partially modified oligonucleotide that hybridizes to at least a portion of an mRNA molecule transcribed from the gene; and
(b) a second oligonucleotide strand, wherein the second oligonucleotide strand comprises a deoxyribonucleic acid that is complementary to at least a portion of the first strand, whereby the first oligonucleotide strand and the second oligonucleotide strand enter the cell and the expression of the gene in the cell is inhibited.

2. The method of claim 1, wherein the fully or partially modified oligonucleotide comprises a modification selected from the group consisting of a internucleoside linkage modification or carbohydrate modification.

3. The method of claim 2, wherein the internucleoside linkage modification comprises one or more phosphorothioate linkages.

4. The method of claim 2, wherein the carbohydrate modification comprises a 2′ modification.

5. The method of claim 4, wherein the 2′ modification is selected from the group consisting of 2′-halo, 2′-O-methyl, 2′-O-ethyl, 2′-O-propyl, 2′-O-butyl; 2′-O-pentyl, 2′-O-(2-methoxyethyl), and 2′-O-[2-[N,N-dimethylamino)oxy]-ethyl].

6. The method of claim 4, wherein the 2′ modification comprises a locked nucleic acid, wherein the locked nucleic acid is characterized by a methylene bridge that connects a 2′-oxygen with a 4′-carbon of a ribose.

7. The method of claim 2, wherein the first oligonucleotide strand has the following general structure:
(2′-O-modified ribonucleotide)ₓ-(deoxyribonucleotide)_{y}-(2′-O-modified ribonucleotide)_{z}.
wherein y is at least 5 and x+y+z equals at least 18 but less than 30.

8. The method of claim 7, wherein when y equals 5, x and z differ by no more than 2.

9. The method of claim 7, wherein y is at least 7.

10. The method of claim 7, wherein the 2′-O-modified ribonucleotide comprises a 2′-O-methyl group.

11. The method of claim 1, wherein the second oligonucleotide strand is shorter than or equal in length to the first oligonucleotide strand.

12. The method of claim 1, wherein the composition further comprises a cationic lipid.

13. The method of claim 12, wherein the composition further comprises a bioconjugate dendrimer, wherein the bioconjugate dendrimer comprises a conjugate moiety coupled to a dendrimeric structure.

14. The method of claim 1, wherein the second oligonucleotide strand is an unmodified deoxyribonucleic acid.

15. The method of claim 1, wherein the second oligonucleotide strand is modified at the 5′-end or the 3′-end to a conjugate moiety.

16. The method of claim 15, wherein the modification comprises the conjugation of a conjugate moiety selected from the group of a cholesterol moiety or a peptide.

17. The method of claim 15, wherein the modification comprises the coupling of the second oligonucleotide to one or more other oligonucelotides and thereby forming a dendrimeric structure, which is further conjugated to a conjugate moiety.

18. A method of enhancing the uptake of a single-stranded oligonucleotide by a cell, the method comprising:
(a) hybridizing to the single-stranded oligonucleotide a deoxyribonucleic acid molecule that is complementary to a subsequence of the single-stranded oligonucleotide to create a double-stranded molecule; and
(b) contacting the cell with the double-stranded molecule, whereby uptake of the single-stranded oligonucleotide by the cell is enhanced.

19. The method of claim 18, wherein the single-stranded oligonucleotide comprises a modification selected from the group consisting of a internucleoside linkage modification and a carbohydrate modification.

20. The method of claim 19, wherein the internucleoside linkage modification comprises one or more phosphorothioate linkages.

21. The method of claim 18, wherein the carbohydrate modification comprises a 2′ modification.

22. The method of claim 21, wherein the 2′ modification is selected from the group consisting of 2′-halo, 2′-O-methyl, 2′-O-ethyl, 2′-O-propyl, 2′-O-butyl; 2′-O-pentyl, 2′-O-(2-methoxyethyl), and 2′-O-[2-[N,N-dimethylamino)oxy]-ethyl].

23. The method of claim 21, wherein the 2′ modification comprises a locked nucleic acid, wherein the locked nucleic acid is characterized by a methylene bridge that connects a 2′-oxygen with a 4′-carbon of a ribose.

24. The method of claim 18, wherein the single-stranded oligonucleotide has the following general structure: (2′-O-modified ribonucleotide)ₓ-(deoxyribonucleotide)_{y}-(2′-O-modified ribonucleotide)_{z}, wherein y is at least 5 and x+y+z equals at least 18 but less than 30.

25. The method of claim 24, wherein when y equals 5, x and z differ by no more than 2.

26. The method of claim 24, wherein y is at least 7.

27. The method of claim 24, wherein the 2′-O-modified ribonucleotide comprises a 2′-O-methyl group.

28. The method of claim 18, wherein the deoxyribonucleic acid molecule is shorter than or equal in length to the single-stranded oligonucleotide.

29. The method of claim 18, wherein the method further comprises the addition of a cationic lipid.

30. The method of claim 29, wherein the methode further comprises the addition of a bioconjugate dendrimer, wherein the bioconjugate dendrimer comprises a conjugate moiety coupled to a dendrimeric structure.

31. The method of claim 18, wherein the deoxyribonucleic acid molecule comprises an unmodified deoxyribonucleic acid.

32. The method of claim 18, wherein the deoxyribonucleic acid molecule further comprises a modification selected from the group consisting of a 5′-end modification and a 3′-end modification.

33. The method of claim 32, wherein the modification comprises a conjugated cholesterol moiety or a conjugated peptide.

34. A composition comprising a dendrimeric bioconjugate and an oligomeric compound, wherein the dendrimeric bioconjugate comprises a conjugate moiety coupled to a dendrimeric structure.

35. The composition of claim 34, wherein the dendrimeric structure is selected from the group of a cationic polymer such as PAMAM and a dendrimeric oligonucleotide structure.

36. The composition of claim 34, wherein the conjugate moiety is selected from the group of lipidic molecules such as cholesterol and peptides.

37. The composition of claim 34, wherein the oligomeric compound comprises a hybridization complex, and wherein the hybridization complex comprises:
(i) a first oligonucleotide strand, wherein the first oligonucleotide strand comprises a fully or partially modified oligonucleotide that hybridizes to at least a portion of an mRNA molecule transcribed from the gene; and
(ii) a second oligonucleotide strand, wherein the second oligonucleotide strand comprises a deoxyribonucleic acid that is complementary to at least a portion of the first strand.

38. The composition of claim 34, further comprising a cationic lipid.

39. A method of enhancing the uptake of an oligomeric compound, the method comprising:
(a) combining the oligomeric compound with a dendrimeric bioconjugate in a composition, wherein the dendrimeric bioconjugate comprises a conjugate moiety coupled to a dendrimeric structure; and
(b) contacting the cell with the composition.

40. A method of inhibiting the expression of a gene in a cell, the method comprising administering to the cell a composition comprising an oligomeric compound and a dendrimeric bioconjugate, wherein the dendrimeric bioconjugate comprises a conjugate moiety coupled to a dendrimeric structure.

41. The method of claim 40, wherein the dendrimeric structure is selected from the group of a cationic polymer such as PAMAM and a dendrimeric oligonucleotide structure.

42. The method of claim 40, wherein the conjugate moiety is selected from the group of lipidic molecules such as cholesterol and peptides.

43. A method for the preparation of cholesterol-dendrimeric oligonucleotide bioconjugate, the method comprising:
(a) derivatizing cholesterol to obtain a free carboxylgroup;
(b) coupling to the derivatized cholesterol Fmoc-lysine-OMe or an analogous structure comprising a protected amino group and a protected carboxylgroup which are separately cleavable;
(c) deprotecting the carboxyl group;
(d) coupling the obtained compound to a solid phase through an acid labile function, like an ester;
(e) deprotecting the amino group;
(f) repetitive steps of
[1] coupling of a compound with two protected amino functions and a free carboxylgroup like (Fmoc)₂-Lys-OH; and
[2] deprotecting the amino-funtion protecting groups according to the amount of dendrimeric units requested;
(g) coupling a linker with a free hydroxyl as reactive group; and
(h) performing standard oligonucleotide synthesis followed by cleaving the compound of from the solid phase.

44. A method of enhancing the uptake in a cell of an oligomeric compound mediating RNA interference such as siRNAs, the method comprising combining the oligomeric compound with a dendrimeric structure such as a cationic polymer.
